(19) United States
(12) Patent Application Publication
(10) Pub. No.: US 2009/0318445 A1
(43) Pub. Date: Dec. 24, 2009

(54) PDF INHIBITORS

(75) Inventors: Arkadius Pichota, Chromos (SG); Jeyaraj Duraiswamy, Chromos (SG); Zheng Yin, Chromos (SG); Thomas Hugo Keller, Chromos (SG); Mark Schreiber, Chromos (SG)

Correspondence Address: NOVARTIS;CORPORATE INTELLECTUAL PROPERTY, ONE HEALTH PLAZA 104/3, EAST HANOVER, NJ, 07936-1080, US

(73) Assignees: NORVARTIS AG, Basel (CH); NOVARTIS PHARMA GMBH, Vienna (AT)

(21) Appl. No.: 12/159,429

(22) PCT Filed: Dec. 28, 2006
(86) PCT No.: PCT/EP2006/070234

§ 371 Date: Jun. 27, 2008

(30) Foreign Application Priority Data
Dec. 30, 2005 (SG) 200508608-7

(51) Int. Cl. A61K 31/5377; C07D 403/02; A61K 31/4164; C07D 471/02; A61K 31/437; C07D 403/14; A61K 31/496; C07D 413/14; C07D 473/00; A61K 31/52; C07D 235/02; C07D 263/54; A61K 31/423; A61P 31/04

(52) U.S. Cl. 514/234.5; 548/306.1; 514/394; 546/118; 514/303; 544/370; 514/254.06; 544/139; 544/264; 514/263.2; 548/302.1; 548/217; 514/375

(57) ABSTRACT

The invention relates to novel compounds that are inhibitors of peptidyl deformylase (PDF). The compounds are useful as antimicrobials and antibiotics. The compounds of the invention display selective inhibition of peptidyl deformylase versus other metalloproteinases such as MMPs. Methods of preparation and uses of the compounds are also disclosed.

### TECHNICAL FIELD

This invention is directed to novel compounds, to the uses of these compounds in various medicinal applications, including treating disorders amenable to treatment by peptidyl deformylase inhibitors, such as treatment of bacterial infections like tuberculosis, and to pharmaceutical compositions comprising these compounds.

### BACKGROUND

Mycobacterium tuberculosis, the causative agent for tuberculosis (TB), infects one-third of the world's population, resulting in nine million new cases of active TB and two million deaths each year (Kremer, et al Expert Opin. Investig. Drugs, 11 (2002), 1033-1049)). TB is presently treated with a four-drug combination (isoniazid, rifampin, pyrazinamide, ethambutol) that imposes a lengthy 6-9 month treatment course, often under the direct observation of a healthcare provider (Davies, et al., Expert Opin. Investig. Drugs, 12 (2003), 1297-1312).

The major shortcoming of this regimen is the long treatment time, which makes patient compliance and proper implementation a challenge. More than two-thirds of the TB patients do not receive full and proper TB treatment, which results in a high relapse rate and emergence of drug resistance. Currently, about 4% of the TB cases worldwide are multiple-drug resistant (MDR) i.e. resistant to both isoniazid and rifampicin. MDR-TB is difficult to cure, with treatment time up to 2 years and a high failure rate. Novel TB drugs are urgently needed to shorten treatment time and to treat multi-drug resistant TB in a more effective way.

Treatment of microbial infection in host organisms requires an effective means to kill the microbe while doing as little harm to the host as possible. Accordingly, agents which target characteristics unique to a pathology-causing microorganism are desirable for treatment. Metalloproteinases are critical to many aspects of normal metabolism. The class known as matrix metalloproteinases (MMPs) are involved in tissue remodelling, such as degradation of the extracellular matrix. Disorders involving metalloproteinases have been implicated in several diseases such as cancer, arthritis, and autoimmune diseases. Because of the importance of MMPs in normal physiological processes, it would be preferable to develop agents that inhibit peptidyl deformylase (PDF) while avoiding significant inhibition of MMPs. Alternatively, PDF inhibitors which also inhibit MMPs may be of use where the therapeutic benefits of inhibiting PDF outweigh the risk of side effects from MMP inhibition. Thus far, compounds using hydroxamic acid or N-formyl hydroxylamine as chelators exhibit appreciable antibacterial activity and in vivo efficacy, including oral activity. N-formyl hydroxylamine derivatives are described in International Patent Application WO 99/39704 and WO 02/102790. As expected, PDF inhibitors can treat infections caused by bacteria resistant to currently available drugs. However, resistance to PDF inhibitors has also been extensively studied (Clements, et al. Antimicrob Agents Chemother 45 (2001), 563-570; Margolis et al., Antimicrob. Agents Chemother. 44 (2000), 1825-1831; and Margolis, et al., Antimicrob Agents Chemother. 45 (2001) 2432-2435). The in vitro frequency of mutation to resistance is low for Streptococcus pneumoniae and Haemophilus influenzae, the two leading respiratory infection pathogens. For Staphylococcus aureus, a higher resistance frequency is observed, but these resistant mutants appear less virulent in vivo.

In view of the importance of identifying new antibiotics to treat bacteria resistant to existing antibiotics, it is desirable to develop novel inhibitors of PDF for evaluation and use as antibacterial and antimicrobial agents. The present invention fulfils this need.

### SUMMARY OF INVENTION

In a first aspect, the invention provides a compound of formula (I) or a pharmaceutically acceptable salt, ester or prodrug thereof:

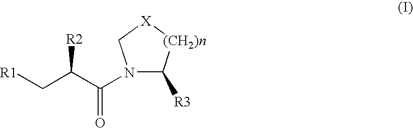

wherein
n is 1 or 2;
X is CH₂, S or CHF;
R1 is —N(OH)CHO or —C(O)NH(OH)
R2 is alkyl, alkylcycloalkyl or alkylaryl or R2 represents a cycloalkyl group, where the carbon adjacent to the carbonyl group forms part of the cycloalkyl ring;
R3 is a substituent of formula (a) or (b), or tetrazolyl, 2-perimidinyl or 4-phenylimidazol-2-yl;

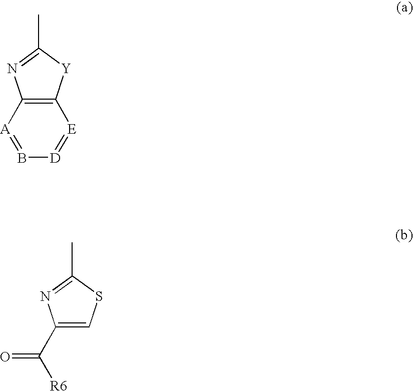

where
Y is NH, O, S or NR4;
A, B, D and E are each independently selected from CH, N, or CR5; or A and E are CH and B and D are fused to and form part of an aryl ring or a 5- or 6-membered nitrogen heterocycle;
R4 is hydroxyalkyl, alkyl or heteroalkyl;
R5 is haloalkyl, heterocyclo optionally substituted with an alkyl group, halogen, alkyl, amino, cyano, nitro, aryl, alkoxy, haloalkoxy, —CO₂R7, —SO₂R8, NHC(O)R9 or —NHSO₂R9; or two R5 groups together form a 6-membered oxygen containing heterocycle, optionally substituted with one or more halogens and fused to the 6-membered ring of substituent (a);
R6 is amino or alkoxy;
R7 is H, alkyl, NHR10, NR10R11 or NH₂;
R8 is aryl, heterocyclo, alkyl or amino;
R9 is heteroaryl or aryl; and
R10 and R11 are each independently an alkyl, alkenyl, alkynyl or aryl group.

Preferably n is 1. It is also preferred that X is CH₂ or CHF. Preferably R1 is —N(OH)—CHO. It is also preferred that R3 is a substituent of formula (a). It is still further preferred that R3 is a substituent of formula (a) and Y is O or NH. More preferably R3 is a substituent of formula (a) and R5 is trifluoromethyl, 4-Me-piperizin-1-yl, fluoro, chloro, methoxy, amino, methyl, cyano, t-butyl, phenyl, nitro, trifluoromethoxy, —SO₂NH₂, —SO₂(morpholino), —SO₂Et, —CO₂Me, —CO₂Et, —NHC(O)(2-pyrazinyl) or —NHSO₂Ph, or two R5 groups together form a substituent (i) or (ii):

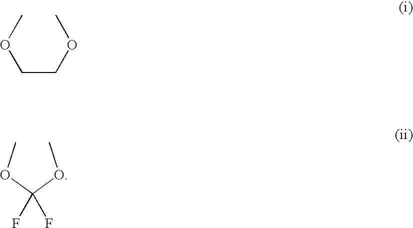

Preferably R2 is lower alkyl, lower alkylcycloalkyl or lower alkyaryl. More preferably R2 is n-propyl, n-butyl, n-pentyl, cyclopentylmethyl or benzyl, or R2 is a cyclohexyl group, where the carbon adjacent to the carbonyl group forms part of the cyclohexyl ring. Most preferably R2 is n-butyl.

It is preferred that B and D are fused to a phenyl ring or a pyrazole ring.

Alternatively it is preferred that R3 is a substituent of formula (b) and R6 is amino or ethoxy.

It is preferred that R4 is heteroalkyl, more preferably an alkyl group having an alkoxy substituent. Most preferably R4 is hydroxyethyl, methoxyethyl or methyl.

In one embodiment, the invention provides a compound of formula (I′), or a pharmaceutically acceptable salt, ester or prodrug thereof:

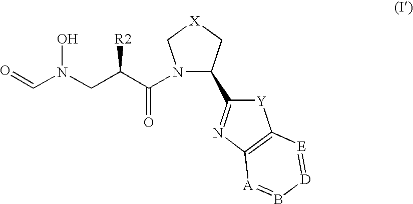

wherein
R2 is n-propyl, n-butyl, n-pentyl, cyclopentylmethyl, or benzyl;
X is CH₂ or CHF;
Y is NH, O or S; and
A, B, D and E are each independently CH, N, or CR5;
where R5 is as defined in claim 10.

Preferably R2 in the compound of formula (I′) is n-butyl. It is also preferred that Y in the compound of formula (I′) is NH or O.

In some preferred embodiments, A in the compound of formula (I) or (I′) is N. It is also preferred that B and E are both N.

Preferably X in the compound of formula (I) or (I′) is CH₂. Alternatively preferably X in the compound of formula (I) or (I′) is CHF.

In another aspect, the invention provides a pharmaceutical composition comprising a compound of formula (I) or (I′) as defined above, or a pharmaceutically acceptable salt, ester or prodrug thereof, in combination with a pharmaceutically acceptable excipient, diluent or carrier.

In still another aspect, the invention provides a method for treating and/or preventing a disease or disorder amenable to treatment by peptidyl deformylase inhibitors comprising administering to a subject in need thereof an effective peptidyl deformylase inhibiting amount of a compound of formula (I) or (I′) as defined above, a pharmaceutically acceptable salt, ester or prodrug thereof.

In yet another aspect, the invention provides the use of an effective peptidyl deformylase inhibiting amount of a compound of formula (I) or (I′) as defined above, a pharmaceutically acceptable salt, ester or prodrug thereof, in the manufacture of a medicament for treating and/or preventing a disease or disorder amenable to treatment by peptidyl deformylase inhibitors.

In another aspect, the invention provides a pharmaceutical composition for treating and/or preventing a disease or disorder amenable to treatment by peptidyl deformylase inhibitors comprising a compound of formula (I) or (I′) as defined above, or a pharmaceutically acceptable salt, ester or prodrug thereof, in combination with a pharmaceutically acceptable excipient, diluent or carrier.

Preferably the disease or disorder is a bacterial infection. More preferably the bacterial infection is a mycobacterial infection. Still more preferably the mycobacterial infection is caused by Mycobacterium tuberculosis. Most preferably the mycobacterial infection is caused by a multidrug resistant form of Mycobacterium tuberculosis.

### DETAILED DESCRIPTION

### Definitions

Unless otherwise stated, the following terms as used in the specification have the following meanings.

The term “aliphatic group” refers to saturated or unsaturated aliphatic groups, such as alkyl, alkenyl or alkynyl, cycloalkyl or substituted alkyl including straight-chain, branched-chain and cyclic groups having from 1-10 carbon atoms.

The term “alkyl” or “alk” as used herein refers to a saturated straight chain or branched aliphatic group of 1-10 carbon atoms. The term “lower alkyl” refers to C₁₋₆alkyl. Preferably, alkyl groups are C₁-C₇alkyl, particularly C₁-C₄alkyl. Examples of “alkyl” or “alk” include, but are not limited to, methyl, ethyl, n-propyl, isopropyl, n-butyl, i-butyl, sec-butyl, t-butyl, n-pentyl, neopentyl, n-hexyl, n-heptyl, cyclopropyl, especially n-butyl.

The term “cycloalkane” or “cycloalkyl” refers to a saturated or partially saturated (non-aromatic) ring comprising preferably 3 to 8 carbon atoms. Examples include cyclopropyl, cyclobutyl, cyclopentyl, cyclohexyl and cycloheptyl. The “cycloalkane” or cycloalkyl” groups preferably contain from 3 to 7 ring carbon atoms.

Any alkyl group as defined above may be substituted with one or more substituents, preferably 1 to 3 substituents, including, but not limited to, substituents such as halogen, lower alkoxy, hydroxy, mercapto, carboxy, cycloalkyl, aryl, heteroaryl, and the like. Examples of substituted alkyl groups include, but are not limited to, haloalkyl groups such as fluoromethyl, difluoromethyl, trifluoromethyl and pentafluoroethyl or other substituted alkyl groups such as hydroxymethyl, 1- or 2-hydroxyethyl, methoxymethyl, 1- or 2-ethoxyethyl, carboxymethyl, 1- or 2-carboxyethyl, and the like.

The term “aryl” or “Ar” refers to an aromatic carbocyclic group of 6 to 14 carbon atoms having a single ring (including, but not limited to, groups such as phenyl) or multiple condensed rings (including, but not limited to, groups such as naphthyl or anthryl), and is especially phenyl.

The term “carbonylamine” as used herein refers to a —NHC(O)— group wherein the amino portion of the group is linked to the aryl/heteroaryl and the carbonyl portion of the group is linked to the azacyclo C₄₋₇ alkane, thiazacyclo C₄₋₇ alkane or imidazacyclo C₄₋₇ alkane.

The term “heteroaryl” or “HetAr” refers to a 4- to 7-membered, monocyclic aromatic heterocycle or a bicycle that is composed of a 4- to 7-membered, monocyclic aromatic heterocycle and a fused-on benzene ring. The heteroaryl has at least one hetero atom, preferably at least two heteroatoms including, but not limited to, heteroatoms such as N, O and S, within the ring. A preferred heteroaryl moiety is a 5- or 6-membered, monocyclic heterocycle having 1, 2, 3 or 4 nitrogen heteroatoms in the ring. Examples of heteteroaryl groups are pyridinyl, pyrimidinyl, pyrazinyl, pyridazinyl, pyridazinyl N-oxide, piperizinyl, benzdioxolanyl, morpholino, triazine, thiazolyl or tetrazolyl.

Any aryl or heteroaryl group may be unsubstituted or substituted by one or more substituents including, but not limited to C₁₋₇ alkyl, particularly C₁₋₄ alkyl such as methyl, hydroxy, alkoxy, acyl, acyloxy, SCN, cyano, nitro, thioalkoxy, phenyl, heteroalkylaryl, alkylsulfonyl, halogen, and formyl.

The term “heteroalkyl” refers to saturated or unsaturated C₁₋₈ alkyl as defined above, and especially C₁₋₄ heteroalkyl which contain one or more heteroatoms, as part of the main, branched, or cyclic chains in the group. Heteroatoms may independently be selected from the group consisting of —NR— where R is hydrogen or alkyl, —S—, —O—, and —P—; preferably —NR— where R is hydrogen or alkyl, and/or —O—. Heteroalkyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, and —CH₂—CH₂—NH—CH₂—CH₂—.

The heteroalkyl group may be unsubstituted or substituted with one or more substituents, preferably one to three substituents, including but not limited to, alkyl, halogen, alkoxy, hydroxyl, mercapto, carboxy, and especially phenyl. The heteroatom(s) as well as the carbon atoms of the group may be substituted. The heteroatom(s) may also be in oxidized form.

The term “alkoxy” as used herein refers to a C₁₋₁₀ alkyl or alkenyl linked to an oxygen atom. Alkoxy is preferably C₁₋₇ alkoxy, more preferably C₁₋₄ alkoxy. Examples of alkoxy groups include, but are not limited to, groups such as methoxy, ethoxy, n-butoxy, tert-butoxy, and allyloxy.

The term “acyl” as used herein refers to the group —C(O)R where R is alkyl, especially C₁₋₇ alkyl such as methyl. Examples of acyl groups include, but are not limited to, acetyl, propanoyl and butanoyl.

The term “acyloxy” as used herein refers to the group —OC(O)R, wherein R is hydrogen, alkyl, especially C₁₋₇ alkyl such as methyl or ethyl, or phenyl or substituted alkyl as defined above.

The term “halogen” or “halo” as used herein refers to chlorine, bromine, fluorine, iodine, and is especially fluorine or chlorine.

The term “thioalkoxy” as used herein means a group —SR where R is an alkyl as defined above, e.g., methylthio, ethylthio, propylthio, butylthio, and the like.

The term “heteroalkylaryl” as used herein means a heteroalkyl group, e.g., —O—CH₂-substituted with an aryl group, especially phenyl. The phenyl group itself may also be substituted with one or more substituents such as halogen, especially fluoro and chloro, and alkoxy such as methoxy.

The term “alkylsulfonyl” as used herein means a group —SO₂R wherein R is alkyl, especially C₁₋₇ alkyl, such as methyl sulfonyl.

The term “alkylcycloalkyl” as used herein means —R-cycloalkyl where R is an alkyl group as defined above. Examples include cyclopentylmethyl.

The term “alkylaryl” as used herein means —R-aryl where R is an alkyl group as defined above. Examples include benzyl.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups may be found in Greene et al., “Protective Groups in Organic Synthesis”, 2nd Ed., John Wiley & Sons, Inc., New York (1991). Preferred amino protecting groups include, but are not limited to, benzyloxycarbonyl (CBz), t-butyl-oxycarbonyl (Boc), t-butyldimethylsilyl (TBDMS), 9-fluorenylmethyl-oxycarbonyl (Fmoc), or suitable photolabile protecting groups such as 6-nitroveratryloxy carbonyl (Nvoc), nitropiperonyl, pyrenylmethoxycarbonyl, nitrobenzyl, dimethyl dimethoxybenzyl, 5-bromo-7-nitroindolinyl, and the like. Preferred hydroxy protecting groups include Fmoc, TBDMS, photolabile protecting groups (such as nitroveratryl oxymethyl ether (Nvom)), Mom (methoxy methyl ether), and Mem (methoxy ethoxy methyl ether). Particularly preferred protecting groups include NPEOC (4-nitrophenethyloxycarbonyl) and NPEOM (4-nitrophenethyloxy-methyloxycarbonyl).

It will be appreciated that the compounds of formula (I) may exist in the form of optical isomers, racemates or diastereoisomers, for example, optical isomers in the R- or S-configuration. It is to be understood that the present invention embraces all enantiomers and their mixtures. Similar considerations apply in relation to starting materials exhibiting asymmetric carbon atoms as mentioned.

The compounds of the invention may exist in the form of solid crystalline salts. Preferably the crystalline salts are metal salts, preferably of divalent metals, although for some compounds it is possible to form crystalline solids by using monovalent counter ions, such as Na. The counter ion is preferably Mg, Ca or Zn.

The compounds of the invention may typically be in the form of a hydrate or a mixed solvate/hydrate. Typically, the crystalline salt of the invention contains about 2 to 8 waters of hydration, more typically about 2 to 6 waters of hydration, and even more typically about 2 to 4 waters of hydration. Thus, the crystalline salt of the invention typically comprises greater than 2% water, more typically about 4 to about 12% water and even more typically about 8 to about 9% water. Solvates may be of one or more organic solvents, such as lower alkyl alcohols, such as methanol, ethanol, isopropanol, butanol or mixtures thereof.

The compounds of the invention, e.g. the compounds of formula (I), may exist in free form or in salt form, e.g. in the form of a pharmaceutically acceptable salt. A “pharmaceutically acceptable salt” of a compound means a physiologically and pharmaceutically acceptable salt that possesses the desired pharmacological activity of the parent compound and does not impart undesired toxicological effects. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzene-sulfonic acid, 2-napthalenesulfonic acid, 4-toluenesulfonic acid, camphorsulfonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or

(2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanol-amine, tromethamine, N-methylglucamine, and the like.

A compound of the invention, e.g. the compounds of formula (I), may act as a prodrug. “Prodrug” means any compound which releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I) wherein a hydroxy, amino, or sulfhydryl group is bonded to any group that may be cleaved in vivo to regenerate the free hydroxyl, amino, or sulfhydryl group, respectively. Examples of prodrugs include, but are not limited to esters (e.g. acetate, formate, and benzoate derivatives), carbamates (e.g. N,N-dimethylamino-carbonyl) of hydroxy functional groups in compounds of formula (I), and the like.

Functional derivatives of compounds of formula (I) include e.g. acid chloride, acid anhydride or an activated ester.

### Compounds of the Invention

The compounds of the present invention can be used for the treatment or prevention of infectious disorders caused by a variety of bacterial and prokaryotic/eukaryotic organisms. The compounds of the present invention are especially useful for treatment of patients infected with Mycobacterium tuberculosis, including strains which are multi-drug resistant. Other, less known and often neglected diseases may also be treated with the compounds of the present invention. Examples include but are not limited to Mycobacterieum avium (often a secondary infection in AIDS patients); Mycobacterium ulcerans (Buruli ulcer). Parasitic diseases caused by eukaryotic protists such as Plasmodium falciparum (malaria), Plasmodium vivax (malaria), Trypanosoma brucei (sleeping sickness), Trypanosoma cruzi (Chagas disease), Leishmania donovani (Kalazar), and Leishmania major (Leishmaniosis), are other diseases which may be treated using the compounds of the present invention.

The compounds of the present invention have optimal PK properties and are particularly suited for chronic treatment. Further, the compounds have a reduced or eliminated inhibition of CYP450 and MMP, as well as a reduced release of aromatic amines in vivo, an important feature in avoiding methemoglobinemia.

The compounds of the invention also preferably have improved safety, toxicity and pharmacokinetic properties, e.g. a decrease or elimination of potential adverse events in human relative to prior art compounds.

The IC₅₀ values of the compounds of formula (I) determined for zinc-containing peptidyl deformylase range from about 0.001 μM to about 0.2 μM. Preferably the IC₅₀ values of the compounds of formula (I) are below 0.2 μM, more preferably below 0.1 μM, still more preferably below 0.05 μM, and most preferably below 0.01 μM. The IC₅₀ values of the compounds of formula (I) determined for nickel-containing peptidyl deformylase range from about 0.005 μM to about 3 μM. Preferably the IC₅₀ values of the compounds of formula (I) are below 3 μM, more preferably below 2 μM, more preferably below 1.5 μM, more preferably below 1 μM, still more preferably below 0.5 μM, still more preferably below 0.1 μM and most preferably below 0.01 μM.

In one aspect, compositions, for treating or preventing infectious disorders are provided, comprising a compound of the invention, a pharmaceutically acceptable salt thereof or a prodrug thereof, as disclosed herein in combination with a pharmaceutically acceptable carrier. In another embodiment, such compositions further include another therapeutic agent.

In another aspect, there is provided a dosage amount of a compound of the invention, a pharmaceutically acceptable salt thereof or a prodrug thereof, as disclosed herein in an effective amount for the treatment, prevention or alleviation of a disorder, such as an infectious disorder. These compounds or derivatives thereof can be screened for activity against different microbial agents and appropriate dosages can be determined using methods available in the art.

The compounds of the invention can be used to treat a subject to treat, prevent, or reduce the severity of an infection. Subjects include animals, plants, blood products, cultures and surfaces such as those of medical or research equipment, such as glass, needles, surgical equipment and tubing, and objects intended for temporary or permanent implantation into an organism. Preferred animals include mammals, e.g., mice, rats, cats, dogs, cows, sheep, pigs, horses, swine, primates, such as rhesus monkeys, chimpanzees, gorillas, and most preferably humans. Treating a subject includes, but is not limited to, preventing, reducing, or eliminating the clinical symptoms caused by an infection of a subject by a microorganism; preventing, reducing, or eliminating an infection of a subject by a microorganism; or preventing, reducing, or eliminating contamination of a subject by a microorganism. The microorganism involved is preferably a prokaryote, more preferably a bacterium or a eukaryotic protist.

In one aspect, methods of treating or preventing an infectious disorder in a subject, such as a human or other animal subject, that are responsive to inhibition of peptidyl deformylase are provided, by administering to the subject an effective peptidyl deformylase inhibiting amount of a compound of the invention, a pharmaceutically acceptable salt thereof or a prodrug thereof. In one embodiment, the compound or its derivative is administered in a pharmaceutically acceptable form optionally in a pharmaceutically acceptable carrier. The compound of the invention, pharmaceutically acceptable salt thereof or prodrug thereof, can be administered alone or in combination with another therapeutic agent. Examples of such therapeutic agents include, but are not limited to, β-lactam, quinolone, macrolide, glycopeptide and oxazolidinone. As used herein, an “infectious disorder” is any disorder characterized by the presence of a microbial infection, such as the presence of bacteria. Such infectious disorders include, for example, tuberculosis and multidrug resistant tuberculosis, central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, antibacterial prophylaxis in immunosuppressed patients, such as patients receiving cancer chemotherapy, or organ transplant patients and chronic diseases caused by infectious organisms, e.g., arteriosclerosis.

The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, nasal, vaginal, rectal, and oral administration. The specific dosage of antimicrobial to be administered, as well as the duration of treatment, can be adjusted as needed.

In another aspect of the present invention, methods are provided for inhibiting peptidyl deformylase. In one embodiment, the method comprises administering to a subject in need thereof an effective peptidyl deformylase inhibiting amount of a compound of formula (I), a pharmaceutically acceptable salt thereof or a prodrug thereof. The terms “subject” and “effective peptidyl deformylase inhibiting amount” are as defined above.

In yet another aspect of the invention, there is also provided the use of a compound of the formula (I), a pharmaceutically acceptable salt thereof or a prodrug thereof in the preparation of a medicament for use in the treatment of diseases mediated by infectious agents expressing biologically active peptidyl deformylase.

### Other Aspects

The present invention also provides pharmaceutical compositions which comprise a bioactive compound of formula (I), a pharmaceutically acceptable salt thereof, or a prodrug thereof, and a pharmaceutically acceptable carrier. The compositions of the invention include those in a form adapted for oral, topical or parenteral use and can be used for the treatment of bacterial infection in a subject such as animals, preferably mammals, more preferably humans. The pharmaceutical compositions can further include another therapeutic agent as described below.

The antibiotic compounds, also referred to herein as antimicrobial compounds, according to the invention can be formulated for administration in any convenient way for use in human or veterinary medicine, by analogy with other antibiotics. Such methods are known in the art (see, e.g., Remington's Pharmaceutical Sciences, Easton, Pa.: Mack Publishing Co.) and are not described in detail herein.

The composition can be formulated for administration by any route known in the art, such as subdermal, inhalation, oral, topical or parenteral. The compositions can be in any form known in the art, including but not limited to tablets, capsules, wafers, fast melts (without wafers), powders, granules, lozenges, creams or liquid preparations, such as oral or sterile parenteral solutions or suspensions. The compounds can also be administered in liposomal, micellar or microemulsion formulations. The compounds can also be administered as prodrugs, where the prodrug administered undergoes biotransformation in the treated mammal to a form which is biologically active.

The topical formulations of the present invention can be presented as, for instance, ointments, creams or lotions, solutions, salves, emulsions, plasters, eye ointments and eye or ear drops, impregnated dressings, transdermal patches, sprays and aerosols, and can contain appropriate conventional additives such as preservatives, solvents to assist drug penetration and emollients in ointments and creams.

The formulations can also contain compatible conventional carriers, such as cream or ointment bases and ethanol or oleyl alcohol for lotions. Such carriers can be present, for example, from about 1% up to about 99% of the formulation. For example, they can form up to about 80% of the formulation.

Tablets and capsules for oral administration can be in unit dose presentation form, and can contain conventional excipients such as binding agents, for example, syrup, acacia, gelatin, sorbitol, tragacanth, or polyvinylpyrollidone; fillers, for example, lactose, sugar, maize-starch, calcium phosphate, sorbitol or glycine; tabletting lubricants, for example, magnesium stearate, talc, polyethylene glycol or silica; disintegrants, for example, potato starch; or acceptable wetting agents, such as sodium lauryl sulphate. The tablets can be coated according to methods well-known in standard pharmaceutical practice.

Oral liquid preparations can be in the form of, for example, aqueous or oily suspensions, solutions, emulsions, syrups or elixirs, or can be presented as a dry product for reconstitution with water or another suitable vehicle before use. Such liquid preparations can contain conventional additives, such as suspending agents, for example, sorbitol, methyl cellulose, glucose syrup, gelatin, hydroxyethyl cellulose, carboxymethyl cellulose, aluminium stearate gel or hydrogenated edible fats; emulsifying agents, for example, lecithin, sorbitan monooleate, or acacia; non-aqueous vehicles (which can include edible oils), for example, almond oil, oily esters such as glycerine, propylene glycol, or ethyl alcohol; preservatives, for example, methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection can be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions can contain, for example, from about 0.1% by weight to about 99% by weight, e.g., from about 10-60% by weight, of the active material, depending on the method of administration. Where the compositions comprise dosage units, each unit will contain, for example, from about 1-1000 mg of the active ingredient.

The dosage of the compound of the invention will vary with the compound employed, the mode of administration, the treatment desired and the disease indicated, as well as other factors such as a patient's age, body weight, general health and sex. For example, the dosage as employed for adult human treatment will range, for example, from about 1-3000 mg per day, for instance 1500 mg per day, depending on the route and frequency of administration. Such a dosage corresponds to about 0.015-50 mg/kg per day. Suitably the dosage is, for example, from about 5-20 mg/kg per day.

Representative pharmaceutical formulations containing a compound of formula (I) are described below.

The present invention also provides a process for preparing a compound of the invention, e.g. a compound of formula (I) which process comprises reacting a compound of formula (A7):

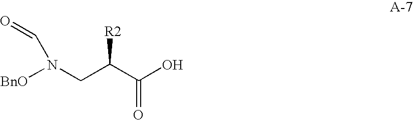

wherein R2 is as defined herein, with a compound of formula (E5):

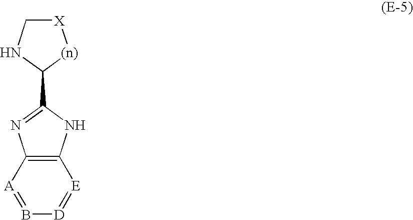

wherein X is CH₂ or CHF;
and A, B, D, E is CH, N, or CR5 as defined herein.

The present invention also provides a process for preparing a compound of the invention, e.g. a compound of formula (I) which process comprises reacting a compound of formula (A7):

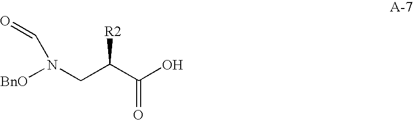

with one of the following compounds:

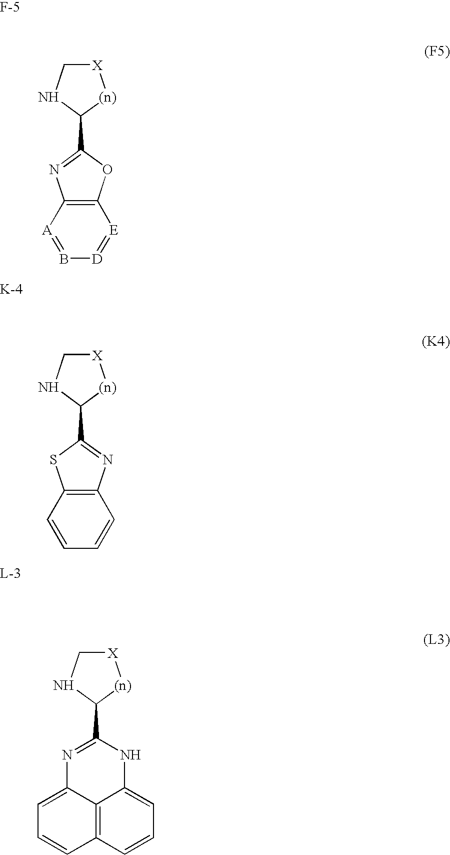

wherein X is CH₂ or CHF;
and A, B, D, E is CH, N, or CR5 as defined herein.

The above reactions may be carried out according to methods known in the art or as disclosed in the Examples below. The reaction may conveniently be carried out in the presence of a base and then followed by hydrogenation, preferably in the presence of a hydrogenation catalyst. Suitable bases include e.g. Hunig base (i.e. diisopropylethylamine) and inorganic bases such as sodium bicarbonate. The hydrogenation catalyst, preferably a palladium catalyst, e.g. palladium on carbon or palladium black, may then be added to the resulting product, e.g. after concentration and stirred under a hydrogen atmosphere e.g. for about 16 to about 24 hours. The palladium catalyst may be added preferably from about 5 mol % to about 10 mol % of the concentrated product.

The invention also contemplates other peptide deformylase inhibitors which are described below.

The compound N-{(R)-2-Cyclopentylmethyl-3-oxo-3-[(S)-2-(1H-tetrazol-5-yl)-pyrrolidine-1-yl]-propyl}-N-hydroxy-formamide having the structural formula:

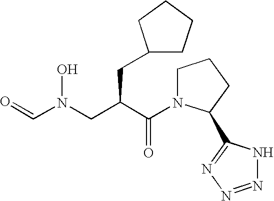

and a process for making the same, which process comprises reacting (R)-2-[(benzyloxy-formyl-amino)-methyl]-3-cyclopentyl-propionic acid A-7 (preparation is described in General Procedure A) and (S)-5-pyrrolidin-2-yl-1H-tetrazole H-2 (preparation is described in General Procedure H) according to General Procedure B.

The compound N-hydroxy-N-{(R)-2-[(S)-2-(1H-tetrazol-5-yl)-pyrrolidine-1-carbonyl]-heptyl}-formamide having the structural formula:

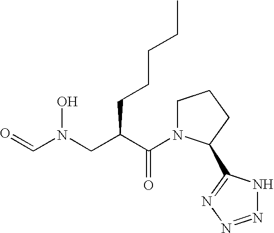

and a method for making the same which comprises reacting (R)-2-[(benzyloxy-formyl-amino)-methyl]-hexanoic acid A-7 (preparation is described in General Procedure A) and (S)-5-pyrrolidin-2-yl-1H-tetrazole H-2 (preparation is described in General Procedure H) according to General Procedure B.

The compound (R)-3-[(S)-2-(1H-benzoimidazol-2-yl)-pyrrolidine-1-carbonyl]-heptanoic acid hydroxyamide having the chemical structure:

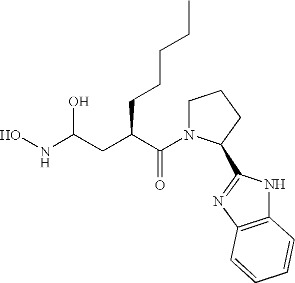

and a process for preparing the same, which process comprises reacting a compound of formula (D1) with a compound of formula (E5) according to General Procedure D.

The compound (R)-3-((S)-2-benzooxazol-2-yl-pyrrolidine-1-carbonyl)-heptanoic acid hydroxyamide having the chemical structure:

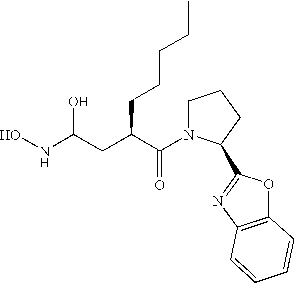

and a process for preparing the same, which process comprises reacting a compound of formula (D1) with a compound of formula (F5) according to General Procedure D.

The compound (R)-3-((S)-2-benzooxazol-2-yl-pyrrolidine-1-carbonyl)-heptanoic acid having the chemical structure:

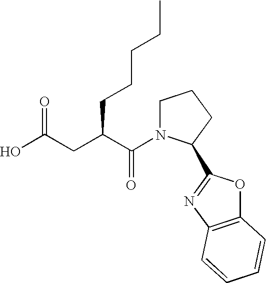

which compound is prepared from (R)-2-Butyl-succinic acid 4-tert-butyl ester D-1 (as described in D) and (S)-2-pyrrolidin-2-yl-benzooxazole F-5 (preparation is described in General Procedure F) according to General Procedure D.

The compound R)-3-((S)-2-benzooxazol-2-yl-pyrrolidine-1-carbonyl)-heptanoic acid methoxy-methyl-amide having the chemical structure:

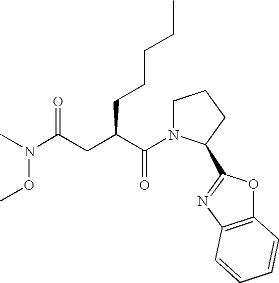

which compound is prepared from (R)-3-((S)-2-Benzooxazol-2-yl-pyrrolidine-1-carbonyl)-heptanoic acid (preparation is described in example 61) and commercially available O,N-dimethyl-hydroxylamine by treatment with EDC/HOBt in DMF.

The compound (R)-3-[(S)-2-(1H-benzoimidazol-2-yl)-pyrrolidine-1-carbonyl]-heptanoic acid amide having the chemical structure:

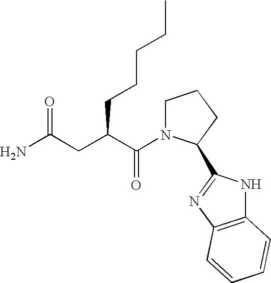

which is prepared by treatment of (R)-3-[(S)-2-(1H-benzoimidazol-2-yl)-pyrrolidine-1-carbonyl]-heptanoic acid (preparation is described in example 60) with ammonia in methanol.

The compound (R)-3-((S)-2-benzooxazol-2-yl-pyrrolidine-1-carbonyl)-heptanoic acid amide, having the chemical structure:

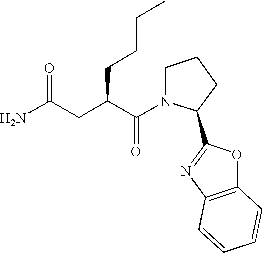

which compound is prepared by treatment of (R)-3-((S)-2-benzooxazol-2-yl-pyrrolidine-1-carbonyl)-heptanoic acid (preparation is described in example 61) with ammonia in methanol.

The compound N-hydroxy-N-{(R)-2-[(S)-2-(5-phenyl-1H-imidazol-2-yl)-pyrrolidine-1-carbonyl]-hexyl}-formamide having the chemical structure:

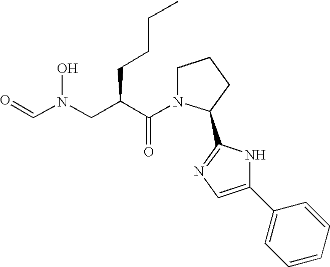

which compound is prepared from (R)-2-[(benzyloxy-formyl-amino)-methyl]-hexanoic acid A-7 (preparation is described in General Procedure A) and 5-Phenyl-2-(S)-pyrrolidin-2-yl-1H-imidazole G-2 (preparation is described in General Procedure G) according to General Procedure B.

The compound N-{(R)-2-cyclopentylmethyl-3-oxo-3-[(S)-2-(5-phenyl-1H-imidazol-2-yl)-pyrrolidine-1-yl]-propyl}-N-hydroxy-formamide having the chemical structure:

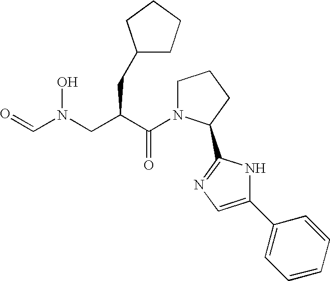

which compound is prepared from (R)-2-[(benzyloxy-formyl-amino)-methyl]-3-cyclopentyl-propionic acid A-7 (preparation is described in General Procedure A) and 5-phenyl-2-(S)-pyrrolidin-2-yl-1H-imidazole G-2 (preparation is described in General Procedure G) according to General Procedure B.

The compound 2-((S)-1-{(R)-2-[(formyl-hydroxy-amino)-methyl]-hexanoyl}-pyrrolidin-2-yl)-thiazole-4-carboxylic acid amide having the chemical structure:

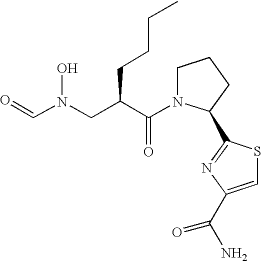

which compound is prepared from (R)-2-[(benzyloxy-formyl-amino)-methyl]-hexanoic acid A-7 (preparation is described in General Procedure A) and (S)-2-Pyrrolidin-2-yl-thiazole-4-carboxylic acid amide J-4 (preparation is described in General Procedure J) according to General Procedure C.

The compound 2-((S)-1-{(R)-2-[(formyl-hydroxy-amino)-methyl]-hexanoyl}-pyrrolidin-2-yl)-thiazole-4-carboxylic acid ethyl ester, which has the chemical structure:

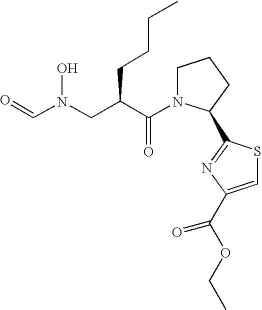

which compound is prepared from (R)-2-[(benzyloxy-formyl-amino)-methyl]-hexanoic acid A-7 (preparation is described in General Procedure A) and (S)-2-pyrrolidin-2-yl-thiazole-4-carboxylic acid ethyl ester J-4 (preparation is described in General Procedure J) according to General Procedure C.

Insofar as the production of starting materials is not particularly described, the starting material compounds are known or may be prepared analogously to methods known in the art or as disclosed in the examples hereinafter.

All patents, patent applications and publications cited in this application are hereby incorporated by reference in their entirety for all purposes to the same extent as if each individual patent, patent application or publication were so individually denoted.

### ABBREVIATIONS

AcOH, HOAc=acetic acid
Ac₂O=acetic anhydride
BOC, Boc=t-butyloxycarbonyl
DCM=dichloromethane
DIEA=diisopropylethylamine
DMF=dimethylformamide
DMSO=dimethylsulfoxide
Et=ethyl
EtOAc=ethyl acetate
Fmoc, FMOC=9-fluorenylmethyloxycarbonyl
HATU=O-(7-aza-benzotriazol-1-yl)-N,N,N′,N′-tetramethyluronium hexafluorophosphate
MCPBA=meta-chloroperoxy-benzoic acid
Me=methyl
Ph=phenyl
MeOH=methanol
MMP=matrix metalloproteinase
NVOM=nitroveratryloxymethyl ether
p-TSA=p-toluenesulfonic acid
RT=room temperature
TFA=trifluoroacetic acid
tBu=t-butyl
THF=tetrahydrofuran
THP=2-tetrahydropyranyl
TsOH or p-TSA=toluenesulfonic acid

### General Synthetic Methods

Compounds of this invention can be made by the methods depicted in the reaction schemes shown below.

The starting materials and reagents used in preparing these compounds are either available from commercial suppliers such as Aldrich Chemical Co., (Milwaukee, Wis., USA), Bachem (Torrance, Calif., USA), Emka-Chemie, or Sigma (St. Louis, Mo., USA) or are prepared by methods known to those skilled in the art following procedures set forth in references such as Fieser and Fieser's Reagents for Organic Synthesis, Volumes 1-15 (John Wiley and Sons, 1991); Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

### Preparation of Compounds of Formula (I)

Compounds of formula (I) can be prepared by methods well known in the art of organic chemistry. Representative synthetic procedures for preparing compounds of the present invention are illustrated and described in detail below. For example, compounds of the present invention may be prepared using the various starting compounds, intermediate compounds or final products as described in Schemes A-N below.

### General Procedure A: Synthesis of 2-[(benzyloxy-formyl-amino)-methyl]-hexanoic acid (A-7)

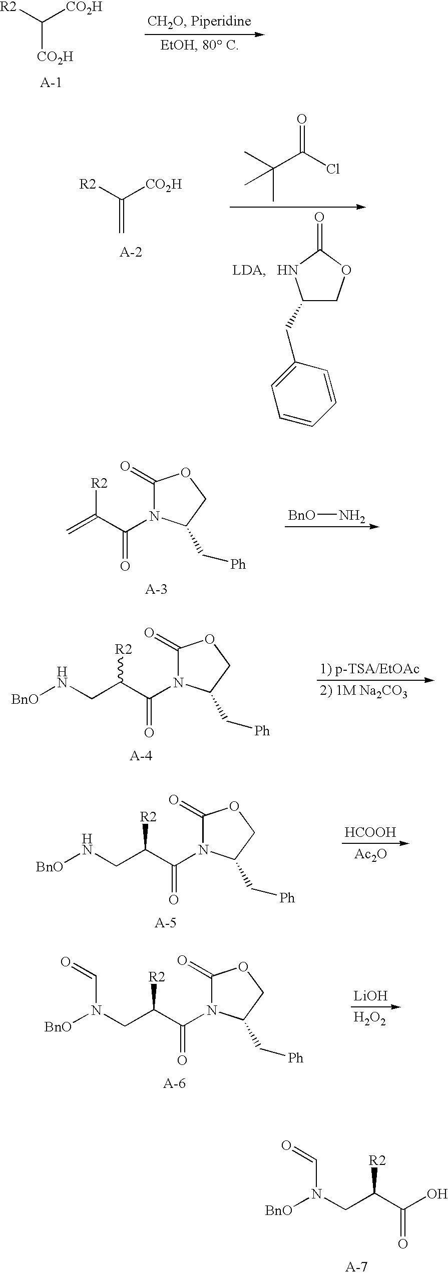

### Step 1: 2-n-butyl acrylic acid (A-2)

To a solution of alkyl malonic acid A-1 (R2=n-butyl (107.4 mmol) in ethanol (200 mL) is added piperidine (12.7 mL, 128.8 mmol, 1.2 equiv.) and 37% aqueous formaldehyde (40.0 mL, 536.9 mmol, 5 equiv.). The solution is heated to 80° C. during which time a precipitate appears, and then gradually redissolves over 1 hour. The reaction mixture is stirred at 80° C. overnight then cooled to room temperature (rt). The solvents are removed under reduced pressure, and the residue is dissolved in ethyl acetate, washed successively with 1 M HCl and brine, dried over anhyd Na₂SO₄, and filtered. The filtrate is concentrated to give the title compound A-2 as a clear oil.

### Step 2: 4-benzyl-3-(2-butyl-acryloyl)-oxazolidin-2-one (A-3)

2-n-Butyl acrylic acid (9.90 g, 77.2 mmol, and 1 equiv.) is dissolved in dry THF (260 mL) and cooled to −78° C. under a blanket of nitrogen. Hunig's base (17.5 mL, 100.4 mmol, 1.3 equiv.) and pivaloyl chloride (9.5 mL, 77.2 mmol, 1 equiv.) are added at such a rate that the temperature remained below −60° C. The mixture is stirred at −78° C. for 30 minutes, warmed to rt for 2 hours, and finally cooled back to −78° C.

In a separate flask, (S)-(−)-4-benzyl-2-oxazolidinone (13.49 g, 77.24 mmol) is dissolved in dry THF (150 mL) and cooled to −78° C. under a blanket of nitrogen. n-Butyllithium (2.5 M solution in hexanes, 30.9 mL, 77.2 mmol, 1 equiv.) is added slowly at −78° C., and the mixture is stirred for 30 minutes at rt. The resulting anion is slowly transferred via a cannula into the original reaction vessel. The mixture is allowed to warm to rt and is stirred overnight at rt. The reaction is quenched with 1 M KHCO₃, and the solvents are removed under reduced pressure. The residue is partitioned between ethyl acetate and water. The organic layer is washed with brine, dried over anhydrous Na₂SO₄, filtered, and concentrated to give a yellow oil which is purified by flash chromatography (hexane:ethyl acetate=4:1) to yield the title compound A-3 as a white solid (15.0 g, 52.2 mmol, 68%).

### Step 3: 4-benzyl-3-[2-(benzyloxyamino-methyl)-hexanoyl]-oxazolidin-2-one (p-toluenesulfonic acid salt)

Compound A-3 (8.25 g, 28.7 mmol) is mixed with O-benzylhydroxylamine (7.07 g, 57.4 mmol, 2 equiv.) and stirred for 40 hours at rt under nitrogen. The mixture is dissolved in ethyl acetate and p-toluenesulfonic acid (21.84 g, 114.8 mmol, and 4 equiv.) is added to precipitate excess O-benzylhydroxylamine as a white solid. The white solid is filtered off, and the filtrate is concentrated to give a crude yellow oil (HPLC analysis indicated a small trace of starting material). Charging the crude yellow oil with excess diethyl ether and cooling to 0° C. for 30 minutes gives a solid which is collected by filtration and dried in vacuo to afford the title compound as a white crystalline solid (single diastereomer).

### Step 4: 4-benzyl-3-[2-(benzyloxyamino-methyl)-hexanoyl]-oxazolidin-2-one (A-5)

To a solution of p-TSA salt (22.9 g, 39 3 mmol) dissolved in ethyl acetate (400 mL), is added 1 M Na₂CO₃ (200 mL, 5 equiv.) and stirred at rt for 30 minutes. The layers are separated, and the aqueous layer extracted with ethyl acetate. The combined organic layers are dried over anhydrous Na₂SO₄, filtered, and concentrated to give the title compound as a pale opaque oil (15.8 g, 38.6 mmol, 98%).

### Step 5: N-[2-(4-benzyl-2-oxo-oxazolidine-3-carbonyl)-hexyl]-N-benzyloxy-formamide (A-6)

A solution of compound A-5 (5.38 g, 13.1 mmol, 1 equiv.) in formic acid (7.4 mL, 196.6 mmol, 15 equiv.) is cooled to 0° C. under a blanket of nitrogen. In a separate flask, formic acid (7.4 mL, 196.6 mmol, 15 equiv.) is cooled to 0° C. under a blanket of nitrogen, and acetic anhydride (2.47 mL, 26.2 mmol, 2 equiv.) is added dropwise. The solution is stirred at 0° C. for 15 minutes. The resulting mixed anhydride is slowly transferred via syringe into the original reaction vessel. The mixture is stirred at 0° C. for 1 hour, then at rt for 3 hours. The mixture is concentrated, taken up in dichloromethane, and washed successively with saturated NaHCO₃ and brine. The organic layer is dried over anhydrous Na₂SO₄, filtered, and concentrated to give an opaque oil which is purified by flash chromatography (hexane:ethyl acetate=2:1 then dichloromethane:acetone=9:1) to yield the title compound as a colorless oil.

### Step 6: 2-[(benzyloxy-formyl-amino)-methyl]-hexanoic acid (A-7)

Compound A-6 (0.163 g, 0.372 mmol, 1 equiv.) is dissolved in THF (4.5 mL) and water (1.5 mL) and cooled to 0° C. Hydrogen peroxide (30% in water, 228 μL, 2.23 mmol, 6 equiv.) is added dropwise followed by the slow addition of a solution of lithium hydroxide (0.019 g, 0.446 mmol, 1.2 equiv.) in water (350 μL). The resulting mixture is stirred at 0° C. for 1.5 hours. The basic reaction mixture is quenched with Amberlite IR-120 resin (H⁺) to pH 4-5 at 0° C. The resin is filtered off and rinsed with ethyl acetate. The mixture is concentrated to remove THF, and then taken up in ethyl acetate. The aqueous layer is separated, and the organic layer dried over anhydrous Na₂SO₄, filtered, and concentrated to give an opaque oil which is purified by flash chromatography (dichloromethane:acetone=4:1 then acetone:methanol=99:1) to yield the title compound A-7 as a colorless oil.

### General Procedure B: Synthesis of N-Hydroxy-N-{(R)-2-[(S)-2-(5-trifluoromethyl-1H-benzoimidazol-2-yl)-pyrrolidine-1-carbonyl]-hexyl}-formamide

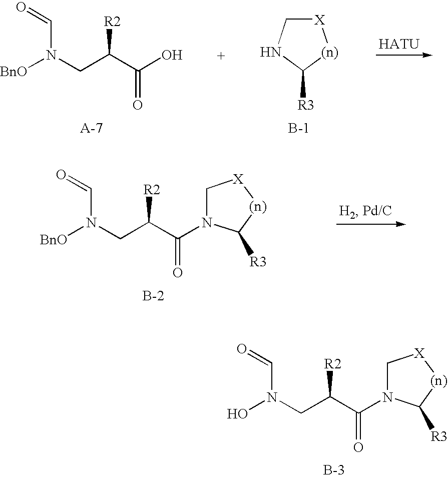

### Step 1: N-Benzyloxy-N-{(R)-2-[(S)-2-(5-trifluoromethyl-1H-enzoimidazol-2-yl)-pyrrolidine-1-carbonyl]-hexyl}-formamide (B-2)

To a mixture of A-7 (0.110 g, 0.39 mmol) and B-1 (0.135 g, 0.53 mmol) in anhydrous DCM at 0° C. under argon DIEA (0.65 ml, 3.94 mmol) followed by HATU (0.225 g, 0.6 mmol) is added. The reaction mixture is stirred overnight while the temp gradually warm up to room temp. The excess DCM is first evaporated under reduced pressure. The remaining crude material is diluted with ethyl acetate and washed with 10% citric acid (2×5 ml) and saturated NaHCO₃ (2×15 ml). The organic layers are dried over anhy MgSO₄, filtered and concentrated under reduced pressure. The residue is purified by silica gel column chromatography to yield the title compound B-2 (0.175 g, 86%).

### Step 2: N-Hydroxy-N-{(R)-2-[(S)-2-(5-trifluoromethyl-1H-benzoimidazol-2-yl)-pyrrolidine-1-carbonyl]-hexyl}-formamide

To a solution of B-2 (0.175 g, 0.34 mmol) in 10 ml EtOH/EtOAc (1:1) is added a catalytic amount of 10% Pd/C (0.018 g, 0.169 mmol) under Argon. Argon is replaced by hydrogen by bubbling through the solvent. After stirring under H₂ atmosphere for about 4 hr, the reaction mixture is filtered over a pad of celite. The celiteis washed several times with EtOH and the combined filtrate is evaporated to dryness via reduced pressure. The residue is purified by silica gel column chromatography to yield B-3 (0.087 g, 53%).

General Procedure C: Synthesis of N—[(R)-2-((S)-2-Benzothiazol-2-yl-pyrrolidine-1-carbonyl)-hexyl]-N-hydroxy-formamide (B-3)

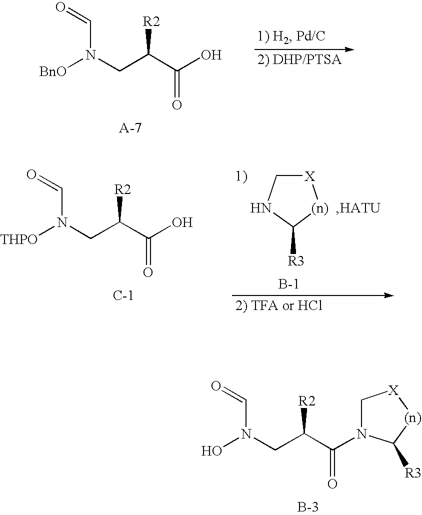

### Step 1: (R)-2-{[Formyl-(tetrahydro-pyran-2-yloxy)-amino]-methyl}-hexanoic acid (C-1)

To a solution of A-7 (6.36 g, 22.8 mmol) in EtOH/EtOAc (1:1) added a catalytic amount of 10% Pd/C (1.2 g, 0.5 eq) under Argon and Argon is replaced by Hydrogen by bubbling through the solvent. After stirring under H₂ atmosphere for about 3 hr, the reaction mixture is filtered over a pad of celite. The celite is washed several times with EtOH and the filtrate collected is evaporated to dryness via reduced pressure to yield the reversed hydroxamate form of A-7. The compound is taken further without any purification. A solution of the reversed hydroxamate (0.714 g, 3.78 mmol) in dichloromethane (12 ml) is cooled to 0° C., then 3,4-dihydro-2H-pyran (0.689 ml, 7.56 mmol) is added followed by p-toluenesulfonic acid monohydrate (0.072 mmol, 0.1 eq). The reaction mixture is stirred at 0° C. for 2 hr. Ice-cold sat. NaCl solution is added to the stirring reaction mixture. The two phases are partitioned with dichloromethane and extracted by excess dichloromethane. The combined organic layers are dried over MgSO₄, filtered and concentrated to give a light brown oil. The crude material is purified by liquid column chromatography to yield 0.752 g, 73% of C1.

### Step 2: N—[(R)-2-((S)-2-Benzothiazol-2-yl-pyrrolidine-1-carbonyl)-hexyl]-N-hydroxy-formamide (B-3)

To a solution of C-1 (0.083 g, 0.275 mmol) in anhydrous dichloromethane (5 ml) and anhydrous DMF (1 ml) is added DIEA (0.3 ml, 1.375 mmol) followed by a solution of B-1 (54 mg, 0.25 mmol) and then the addition of HATU (105 mg, 0.275 mmol). The reaction mixture is stirred overnight under Argon. Upon reaction completion, the excess dichloromethane is evaporated via reduced pressure, followed by addition of water and ethyl acetate to the reaction mixture. The two layers are partitioned and extractions are again carried out on the aqueous layer with more ethyl acetate. The combined organic layers are washed sequentially with 10% citric acid, sat. NaHCO₃, dried over MgSO₄, filtered and concentrated. Purification with flash chromatography yielded the protected compound (0.094 g, 82%). The removal of the protecting group i performed by stirring the obtained material (0.094 g, 0.2 mmol) in 30% TFA in dichloromethane (8 ml) for about 2 hr. The excess solvents are evaporated via reduced pressure to yield crude B-3. Final purification with preparative HPLC yields B-3 (20 mg, 27%).

### General Procedure D: Synthesis of 3-[2-(1H-Benzoimidazol-2-yl)-Pyrrolidine-1-carbonyl]-Heptanoic Acid Hydroxyamide (D-4)

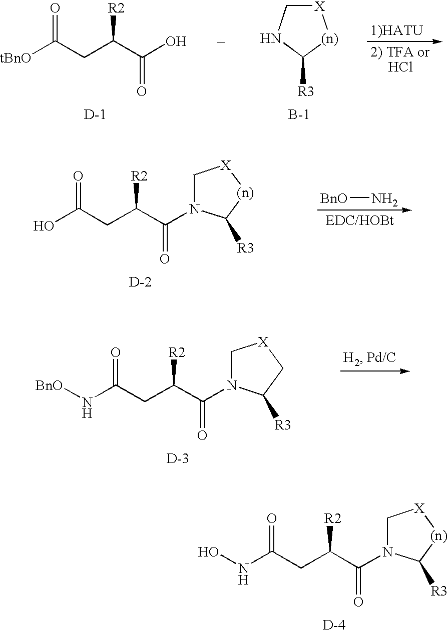

### Step 1: 3-[2-(1H-Benzoimidazol-2-yl)-pyrrolidine-1-carbonyl]-heptanoic acid (D-2)

2-(2-oxo-propyl)-hexanoic acid (D-1, 160 mg, 0.70 mmol) (preparation as in General Procedure E) is dissolved in anhydrous DMF (5 ml) and stirred under argon protection at rt. HATU (400 mg, 1.04 mmol, 1.5 eq) is then added and stirred at rt for about 10 minutes. 2-pyrrolidin-2-yl-1H-Benzoimidazole (B-1, 170 mg, 0.90 mmol, 1.3 eq) is then added to the mixture and stirring is continued for another 10 minutes at rt before DIEA (1.28 ml, 6.9 mmol, 10 eq) is added via a syringe. The reaction mixture is stirred for 2 hours at rt and then tested for reaction completion by TLC and LC-MS. When the reaction is completed, citric acid (5%, 15 ml) is added with stirring, CH₂Cl₂ is added and the phase separated. The aqueous layer is extracted with CH₂Cl₂ (3×) and the combined organic layers are washed with 5% of citric acid, saturated sodium bicarbonate solution, brine and dried over Magnesium Sulfate. After filtration the solvent is removed under reduced pressure to get a crude product as brown oil which is purified by flash column chromatography (Methanol:CH₂Cl₂=1:9) to get 3-[2-(1H-Benzoimidazole-2-yl)-pyrrolidino-1-carbonyl]-heptanoic acid tert-butyl ester as light brown solid (210 mg, 0.53 mmol, 75%).

3-[2-(1H-Benzoimidazol-2-yl)-pyrrolidino-1-carbonyl]-heptanoic acid tert-butyl ester (210 mg, 0.53 mmol) is dissolved in TFA in CH₂Cl₂ (7.5 ml of TFA in 2.5 ml of CH₂Cl₂), the reaction mixture is stirred at rt for 3 hours and then tested for reaction completion by TLC and LC-MS. When the reaction is completed, the solvent is removed under reduced pressure to yield an orange oil which is purified by flash column chromatography (Methanol:CH₂Cl₂=1:9) to get the title compound (D-2) as white solid (150 mg, 0.44 mmol, 83%).

### Step 2: 3-[2-(1H-Benzoimidazol-2-yl)-pyrrolidine-1-carbonyl]-heptanoic acid benzyloxy-amide (D-3)

D-2 (150 mg, 0.44 mmol, 1 equivalent) is dissolved in anhydrous DMF (5 ml) and stirred under argon protection at rt. HOBt (89.18 mg, 0.66 mmol, 1.5 equivalent) and EDC (126.52 mg, 0.66 mmol, 1.5 equivalent) are successively added and stirred at rt for ca. 5 minutes. O-Benzylhydroxylamine Hydrochloride (91.30 mg, 0.57 mmol, 1.3 equivalent) is then added under stirring to the mixture and stirring is continued for another 10 minutes at rt before DIEA (0.41 ml, 2.20 mmol, 5 equivalent) is added via a syringe. The reaction mixture is stirred overnight at rt and then tested for reaction completion by TLC and LC-MS. When the reaction is completed, citric acid (5%, 15 ml) is added with stirring, CH₂Cl₂ is added and the phase separated. The aqueous layer is extracted with CH₂Cl₂ (3×) and the combined organic layers are washed with citric acid (5%), saturated sodium bicarbonate solution, brine and dried over Magnesium Sulfate, filtered and concentrated to get a brown oil which is further purified by column chromatography (Methanol:CH₂Cl₂=1:9) to yield the title compound (D-3) as brown solid (89.6 mg, 0.19 mmol, 44%).

### Step 3: 3-[2-(1H-Benzoimidazole-2-yl)-pyrrolidine-1-carbonyl]-heptanoic acid hydroxyamide (D-4)

D-3 (89.6 mg, 0.19 mmol, 1 eq) is dissolved in ethanol and ethyl acetate (1:1) under argon protection at rt and added to a slurry of Pd/C (10%, 10 mg, 0.10 mmol, 0.5 eq) in ethanol and ethyl acetate (1 ml, 1:1) under Argon. Hydrogen gas is bubbled through the mixture and the reaction mixture is stirred under hydrogen atmosphere for 6 hours and then tested for reaction completion by TLC and LC-MS. When the reaction is completed, the mixture is filtered through celite, the celite is washed with 50 ml of ethanol, and the ethanol solution is concentrated under reduced pressure to a light brown oil which is purified first by column chromatography (Methanol:CH₂Cl₂=2:8) then by preparative reversed phase HPLC to yield the title compound (D-4) as white crystals (44.2 mg, 0.12 mmol, 65%).

### General Procedure E: Synthesis of 2-((2S,4R)-4-Fluoro-pyrrolidin-2-yl)-1H-imidazo[4,5-c]pyridine (E-5)

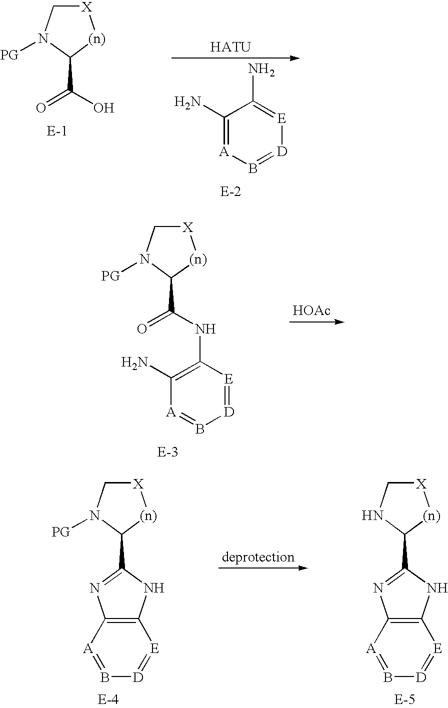

### Step 1: (2S,4R)-2-(3-Amino-pyridin-4-ylcarbamoyl)-4-fluoro-pyrrolidine-1-carboxylic acid tert-butyl ester (E-3)

To a mixture of E-1 (1.17 g, 5.0 mmol) and E-2 (0.71 g, 6.5 mmol) in anhydrous DMF/DCM (1:1) at 0° C. under argon DIEA (4.0 ml, 5.0 eq) followed by HATU (2.47 g, 1.2 eq) is added. The reaction mixture is stirred for another 4 hr while the temp gradually warm up to room temp. LCMS shows that the reaction is completed. The excess DCM is first evaporated via reduced pressure. The remaining crude material is diluted with ethyl acetate and partitioned with water. Extractions are repeated twice with more ethyl acetate. The combined organic layers are dried over anhy Na₂SO₄, filtered and concentrated. The crude is purified via liquid column chromatography and yields the desired product.

### Step 2: (2S,4R)-4-Fluoro-2-(1H-imidazo[4,5-c]pyridine-2-yl)-pyrrolidine-1-carboxylic acid tert-butyl ester (E-4)

E-3 (1.3 g, 4.0 mmol) is dissolved in 40 mL glacial acetic acid and refluxed at 60° C. overnight. The acid is then evaporated via reduced pressure. Purification of the crude material is carried out by flash master to yield the pure E-4 (0.693 g, 57%).

### Step 3: 2-((2S,4R)-4-Fluoro-pyrrolidin-2-yl)-1H-imidazo[4,5-c]pyridine (E-5)

Removal of the tert-butyl protecting group on E-4 (0.346, 1.13 mmol) is carried out by stirring E-4 (346 mg, 1.13 mmol) in a solution of 4N HCl in 1,4-dioxane (5 ml). The reaction mixture is stirred for about 30 min before the excess solvents are evaporated under reduced pressure to yield the target molecule E-5.

### General Procedure F: Synthesis of 2-Pyrrolidin-2-yl-oxazolo[4,5-b]pyridine (F-5)

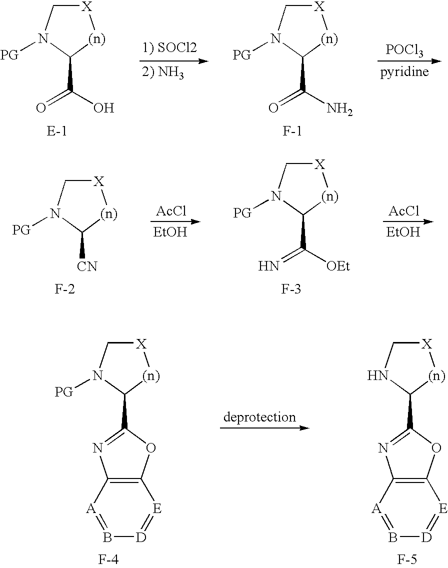

### Step 1: 2-{1-[2-(tert-Butyl-dimethyl-silanyloxy)-ethyl]-1H-benzoimidazol-2-yl}-pyrrolidine-1-carboxylic acid benzyl ester (F-1)

Pyrrolidine-1,2-dicarboxylic acid 1-benzyl ester E-1 (10.0 g, 40 mmol) is dissolved in CH₂Cl₂ (40 ml) and cooled under ice. SOCl₂ (29.3 ml, 400 mmol, 10 equiv.) is added slowly and the mixture is heated at 50° C. for 2 h. After cooling to rt the solvent is removed under reduced pressure to give a slightly yellow oil. The oily intermediate is dissolved in THF (25 ml), cooled to 0° C., and ammonia solution (38 ml, 96 mmol, 2.3 equiv.) is added. The mixture is stirred at rt for 1 hour and the solvent is removed under reduced pressure. The residue is partitioned between CH₂Cl₂ (150 ml) and water (30 ml). The layers are separated, and the organic phase is washed with brine, dried over MgSO₄, filtered, concentrated under reduced pressure to yield 2-Carbamoyl-pyrrolidine-1-carboxylic acid benzyl ester F-1 as a yellowish oil (12.0 g).

### Step 2: 2-Cyano-pyrrolidine-1-carboxylic acid benzyl ester (F-2)

2-{1-[2-(tert-Butyl-dimethyl-silanyloxy)-ethyl]-1H-benzoimidazol-2-yl}-pyrrolidine-1-carboxylic acid benzyl ester F-1 (12.0 g) is dissolved in pyridine (30 ml) and cooled under ice. POCl₃ (3.6 ml) is added slowly and the mixture is stirred at 0° C. for 1 hour. The pyridine is removed under reduced pressure, the crude mixture is dissolved in ethyl acetate (150 ml) and washed with 1N HCl solution (3×30 ml), concentrated under reduced pressure, dissolved in ethyl acetate (50 ml) again, washed with 1N HCl (50 ml) solution to completely remove the pyridine. The ethyl acetate solution is dried over MgSO₄, filtered, concentrated under reduced pressure to yield 2-Cyano-pyrrolidine-1-carboxylic acid benzyl ester F-2 as a slightly yellow oil (6.1 g, 26.5 mmol, 66%).

### Step 3: 2-Ethoxycarbonimidoyl-pyrrolidine-1-carboxylic acid benzyl ester (F-3)

A mixture of EtOH (15 ml) and CH₂Cl₂ (20 ml) is cooled to 0° C. and CH₃COCl is added slowly, followed by a solution of 2-Cyano-pyrrolidine-1-carboxylic acid benzyl ester F-2 (2.0 g, 8.7 mmol) in CH₂Cl₂ (20 ml). The reaction is stirred at rt for 2 hours and the solvent is removed under reduced pressure to give 2-ethoxycarbonimidoyl-pyrrolidine-1-carboxylic acid benzyl ester F-3 as a sticky solid.

### Step 4: 2-Oxazolo[4,5-b]pyridin-2-yl-pyrrolidine-1-carboxylic acid benzyl ester (F-4)

2-Ethoxycarbonimidoyl-pyrrolidine-1-carboxylic acid benzyl ester F-3 is dissolved in EtOH (15 ml) and 2-Amino-pyridin-3-ol (1.1 g, 9.6 mmol, 1.1 equiv) is added. The mixture is heated at 95° C. for 16 hours, then it is cooled to rt and the solvent is removed under reduced pressure. The crude product is purified by flash chromatography to yield the title compound as pale opaque oil (1.2 g, 3.7 mmol, 43%).

### Step 5: 2-Pyrrolidin-2-yl-oxazolo[4,5-b]pyridine (F-5)

2-Oxazolo[4,5-b]pyridin-2-yl-pyrrolidine-1-carboxylic acid benzyl ester F-4 (330 mg, 1.0 mmol) is dissolved in ethyl acetate/MeOH solution (1:1, 15 mL) and added slowly to 10% Pd/C (65 mg, 0.2 equiv.). H₂ gas is bubbled through the mixture for 6 hr until LC-MS shows the reaction is completed. Pd/C is filtered off and the filtrate is concentrated to dryness to yield the title compound 2-Pyrrolidin-2-yl-oxazolo[4,5-b]pyridine F-5 as a slightly yellow oil (143.6 mg, 0.76 mmol, 76%).

### General Procedure G: Synthesis of 5-phenyl-2-pyrrolidin-2-yl-1H-imidazole (G-2)

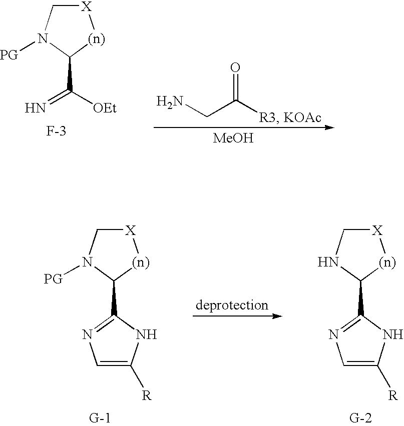

### Step 1: 2-(5-phenyl-1H-imidazol-2-yl)-pyrrolidine-1-carboxylic acid benzyl ester (G-1)

To a solution of compound F-3 (1.00 g, 3.62 mmol, 1 equiv.) dissolved in methanol (20 mL), is added potassium acetate (1.42 g, 14.5 mmol, 4 equiv.) and the mixture is heated to 78° C. under a blanket of argon. A solution of 2-aminoacetophenone (R=Ph) (1.24 g, 7.24 mmol, 2 equiv.) in methanol (10 mL) is added dropwise to the reaction mixture over a period of 3 minutes. The reaction mixture is stirred at 78° C. for 17 hours and allowed to cool to rt. The mixture is concentrated under reduced pressure, taken up in dichloromethane, and washed successively with saturated NaHCO₃. The organic layer is dried over anhydrous MgSO₄, filtered, and concentrated under reduced pressure to give a brown residue.

The residue is triturated with ether, and filtered. The filtrate is concentrated to give a brown residue which is then triturated with ice-cold acetonitrile, and filtered. The solid is further triturated with ether, and filtered. The combined filtrates from both ether and acetonitrile triturations are concentrated to give a brown solid which is purified by flash chromatography (hexane:ethyl acetate=1:1) to yield the title compound G-1 as a brown solid (0.505 g, 1.45 mmol, 40%).

Literature references: Breslin, H. J., Miskowski, T. A., Kukla, M. J., Leister, W. H., De Winter, H. L., Gauthier, D. A., Somers, M. V. F., Peeters, D. C. G., Roevens, P. W. M. J. Med. Chem., 2002, 45, 5303-5310.

### Step 2: 5-phenyl-2-pyrrolidin-2-yl-1H-imidazole (G-2)

Compound G-1 (0.50 g, 1.44 mmol, 1 equiv.) and 10% palladium on charcoal (16 mg) in methanol (15 mL) is stirred under a blanket of hydrogen at rt for 46 hours. The reaction mixture is filtered through celite and the filtrate is concentrated to give a brown residue which is purified by flash chromatography (dichloromethane:methanol:aqueous ammonia=1:9:few drops) to yield the title compound G-2 as a yellow solid (0.2103 g, 0.99 mmol, 98

### General Procedure H: Synthesis of 5-pyrrolidin-2-yl-1H-tetrazole (H-2)

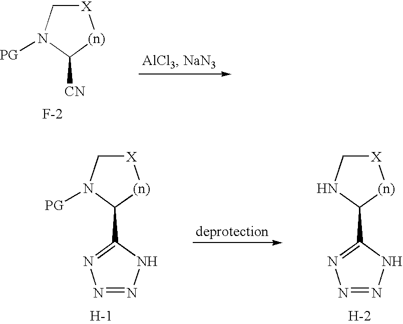

### Step 1: 2-(1H-Tetrazol-5-yl)-pyrrolidine-1-carboxylic acid benzyl ester (H-1)

To a solution of compound F-2 (PG=Cbz) (0.377 g, 1.64 mmol, 1 equiv.) in DMF (1.5 mL) is added sodium azide (0.111 g, 1.71 mmol, 1.04 equiv.) and ammonium chloride (0.097 g, 1.80 mmol, 1.1 equiv.). The solution is heated to 90-95° C. under a blanket of argon and stirred for 4 hours. The reaction mixture is poured onto ice (10 g) and acidified to pH 2 with 1 M HCl. The acidic reaction mixture extracted with dichloromethane and the layers are separated. The combined organic layers are washed successively with water and brine. The organic layer is dried over anhydrous MgSO₄, filtered, and concentrated to give the title compound H-1 as a colourless oil (0.420 g, 1.54 mmol, 94%).

Literature reference: Almquist, R. G., Chao, W. R., Jennings-White, C. J. Med. Chem., 1985, 28, 1067

### Step 2: 5-Pyrrolidin-2-yl-1H-tetrazole (H-2)

Compound H-1 (0.401 g, 1.49 mmol, 1 equiv.) and 10% palladium on charcoal (16 mg) in ethyl acetate/ethanol (1:1, 5 mL) is stirred under a blanket of hydrogen at rt for 4 hours. The reaction mixture is filtered through celite and the filtrate is concentrated to give the title compound H-2 as a white solid (0.168 g, 1.21 mmol, 81%) which is only soluble in presence of acetic acid.

### General Procedure J: Synthesis of 4-Phenyl-2-(S)-pyrrolidin-2-yl-thiazole (J-4)

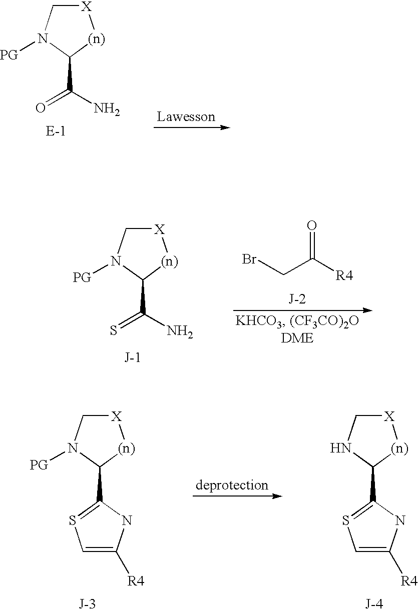

### Step 1: (S)-2-Thiocarbamoyl-pyrrolidine-1-carboxylic acid tert-butyl ester (J-1)

E-1 (1.0 g, 4.7 mol) is dissolved in 20 ml anhydrous DME and added to another round bottom flask containing a slurry of Lawesson's reagent (1.89 g, 4.7 mol) in anhydrous DME (5 ml) under Argon at room temp. The reaction mixture is refluxed at 80° C. for about 2.5 hr and the reaction completion is monitored by LCMS. The excess solvent is evaporated under reduced pressure and the crude residue obtained is kept at low temp away from direct light. The crude material is purified by liquid column chromatography to yield the desired product J-1 (50%).

### Step 2: (S)-2-(4-Phenyl-thiazol-2-yl)-pyrrolidine-1-carboxylic acid tert-butyl ester (J-3)

A mixture of J-1 (1.92 mmol) and potassium bicarbonate (15.4 mmol) are stirred vigorously in anhydrous DME (15 ml). After stirring at room temp for 8 min J-2, (5.77 mmol) is added and stirring is continued for 45 min. The reaction mixture is cooled to 0° C. and trifluoroacetic anhydride (0.7 ml, 7.69 mmol) is added dropwise to the reaction mixture and stirring is continued for another 1 hr at 0° C. The mixture is allowed to slowly warm up to room temp. The excess solvent is evaporated and the residue is partitioned between water and ethyl acetate. The organic layer is collected and extraction is repeated twice more with ethyl acetate. The combined organic layers are dried over anhydrous Na₂SO₄, filtered and concentrated via reduced pressure. The crude material is purified by liquid column chromatography to yield the desired product J-3 (62%).

### Step 3: 4-Phenyl-2-(S)-pyrrolidin-2-yl-thiazole (J-4)

To a solution of J-3 (1.18 mmol) in dichloromethane (15 ml) trifluoroacetic acid (4.5 ml) is added dropwise at 0° C. The reaction mixture is stirred for about 2 hr at low temp, then slowly warmed up to room temp. The excess solvent is evaporated via reduced pressure and yielded the crude material. The crude is purified by liquid column chromatography to yield the desired product J-4 (20%).

### General Procedure K: Synthesis of (S)-2-Pyrrolidin-2-yl-benzothiazole (K-4)

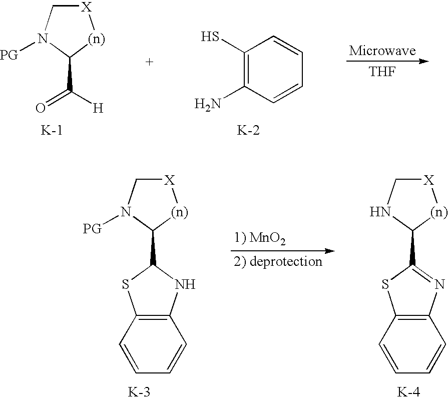

### Step 1: (S)-2-(2,3-Dihydro-benzothiazol-2-yl)-pyrrolidine-1-carboxylic acid tert-butyl ester (K-3)

A mixture of K-1 (0.400 g, 2.0 mmol) and K-2 (432 uL, 4.0 mmol) in dry THF is subjected to microwave at 100° C. for 10 min. The solvent is evaporated to dryness to yield the product K3 (0.612 g). The crude compound K-3 is taken further without any purification.

### Step 2: (S)-2-Pyrrolidin-2-yl-benzothiazole (K-4)

To a solution of K-3 (0.612 mg, 0.31 mmol) from the previous step in toluene (20 ml) is added MnO₂ (8 g, 100 mmol) and the mixture is refluxed at 120° C. under Argon overnight. The MnO₂ is filtered over a pad of celite to give a brownish oil upon solvent evaporation. Then residue is dissolved in Ethyl acetate (20 ml) and washed three times with 1N HCl solution to remove the by-products formed during the reaction. The organic phase collected from the work-up is evaporated to dryness under vacuum to yield the desired product K-4. The crude material is purified by silica gel column chromatography N-Boc-protected K-4 (0.214 g, 35%). N-Boc-proteced K-4 (0.109 g) is deprotected by stirring in a solution of 4N HCl in 1,4-dioxane (1.5 ml) for 2 hr at rt. The solvent is removed under reduced pressure to yield the title compound K-4 (0.073 g).

### General Procedure L: Synthesis of 2-pyrrolidin-2-yl-1H-perimidine (L-2)

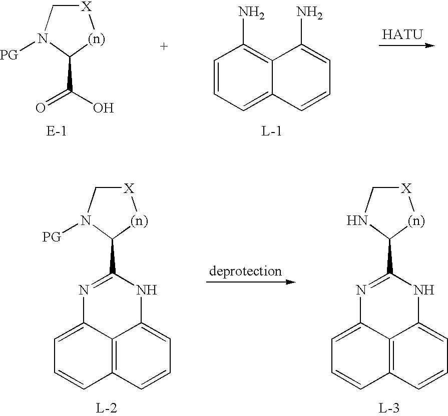

### Step 1: 2-(1H-perimidin-2-yl)-pyrrolidine-1-carboxylic acid benzyl ester (L-2)

N-(benzyloxycarbonyl)-L-proline E-1 (2.00 g, 8.00 mmol, 1 equiv.) is dissolved in DMF (30 mL) and cooled to 0° C. and stirred under a blanket of argon. HATU (3.65 g, 9.60 mmol, 1.2 equiv.), 1,8-diaminonapthalene L-1 (1.51 g, 9.60 mmol, 1.2 equiv.) and DIEA (6.63 mL, 40.0 mmol, 5 equiv.) are added to the solution at 0° C. The reaction mixture is allowed to warm to rt and stirred for 1 hour. The mixture is concentrated, taken up in dichloromethane and washed successively with 5% citric acid and saturated NaHCO₃. The organic layer is dried over anhydrous MgSO₄, filtered, and concentrated to give a brown oil which is purified by flash chromatography (dichloromethane:methanol=49:1) to yield the title compound L-2 as a brown solid (2.93 g, 7.89 mmol, 98%).

### Step 2: 2-pyrrolidin-2-yl-1H-perimidine (L-2)

Compound L-2 (2.93 g, 7.90 mmol, 1 equiv.) and 10% palladium on charcoal (84 mg) in ethyl acetate/ethanol (1:1, 50 mL) is stirred under a blanket of hydrogen at rt for 15 hours. The reaction mixture is filtered through celite and the filtrate is concentrated to give a brown residue which is purified by flash chromatography (hexane:ethyl acetate=1:1 then dichloromethane:methanol=9:1) to yield the title compound H-2 as a yellow solid (0.613 g, 2.58 mmol, 53%).

### General Procedure M: Synthesis of 1-[(benzyloxy-formyl-amino)-methyl]-cyclohexanecarboxylic acid (M-5)

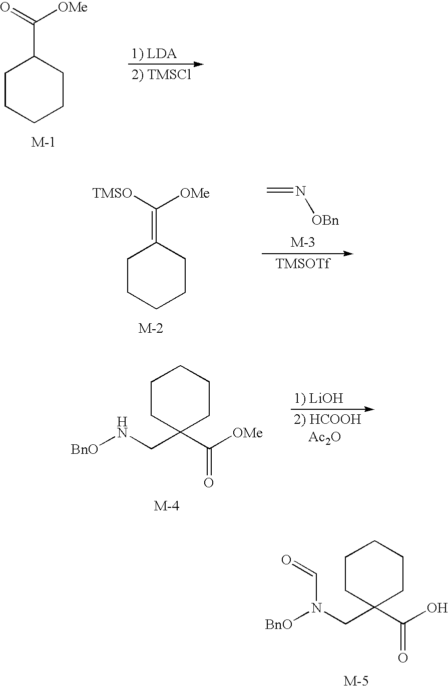

### Step 1: Cyclohexylidene-methoxy-methoxy-trimethyl-silane (M-2)

Lithium diisopropylamine (2 M solution in THF, 35.2 mL, 70.3 mmol, 2 equiv.) is diluted in THF (70 mL) and cooled to −78° C. Cyclohexanecarboxylic acid methyl ester M-1 (5.00 g, 35.2 mmol, 1 equiv.) is added dropwise. The solution is stirred at −78° C. for 30 minutes followed by the addition of trimethylchlorosilane (8.88 mL, 70.3 mmol, 2 equiv.). The resulting mixture is stirred at −78° C. for 30 minutes, then at rt for 17 hours during which time a white precipitate appears and fuming occurs and then fuming gradually ceases over 17 hours. The mixture is concentrated under reduced pressure, taken up in hexane and filtered. The filtrate is concentrated to give an orange oil which is purified by distillation (110° C., 10 mbar) to yield the title compound M-2 as a clear oil (4.673 g, 21.8 mmol, 62%).

Literature references: (1) Ikeda, K., Achiwa, K., Sekiya M., Tetrahedron Lett., 1983, 24, 4707-4710; (2) Maslak, V., Matović, R., Sai\check{c}ić, R. N., Tetrahedron, 2004, 60, 8957-8966.

### Step 2: Formaldehyde-O-Benzyl-Oxime (M-3)

To a solution of a 36.6% aqueous solution of formaldehyde (2.21 mL, 27.0 mmol, 1 equiv.) in water (15 mL) is added pyridine (6.50 mL, 81.0 mmol, 3 equiv.) and O-benzylhydroxylamine hydrochloride (4.31 g, 27.0 mmol, 1 equiv.). The mixture is heated to 70° C. for 1 hour, and allowed to cool to rt. The mixture is acidified with 3N HCl (15 mL) and extracted with ether. The organic layers are combined, dried over anhydrous MgSO₄, filtered and concentrated to give a colorless oil which is purified by flash chromatography (hexane:ethyl acetate=4:1) to yield the title compound M-3 as a colorless oil (3.00 g, 22.2 mmol, 82%).

### Step 3: 1-(Benzyloxyamino-methyl)-cyclohexanecarboxylic acid methyl ester (M-4)

To a solution of compound M-3 (0.636 g, 4.70 mmol, 1 equiv.) dissolved in dichloromethane (15 mL), is added compound M-2 (1.00 g, 4.70 mmol, 1 equiv.), cooled to 0° C. and stirred under a blanket of argon. Trimethylsilyl triflate (0.085 mL, 0.47 mmol, 0.1 equiv.) is added dropwise at 0° C. The reaction mixture is allowed to warm up to rt and stirred for 5 hours. The mixture is washed with 10% potassium carbonate (50 mL), dried over anhydrous MgSO₄, filtered and concentrated to give a yellow oil which is purified by flash chromatography (hexane:ethyl acetate=9:1) to yield the title compound M-4 as a clear oil (0.961 g, 3.46 mmol, 74%).

Literature reference: Maslak, V., Matović, R., Sai\check{c}ić, R. N., Tetrahedron, 2004, 60, 8957-8966.

### Step 3: 1-[(benzyloxy-formyl-amino)-methyl]-cyclohexanecarboxylic acid (M-5)

Compound M-5 (0.166 g, 0.60 mmol, 1 equiv.) is dissolved in THF (6 mL) and lithium hydroxide (3 N solution in water, 2 mL, 10 equiv.) is added dropwise under a blanket of argon, and the mixture is stirred at rt for 5 hours. The reaction mixture is heated to 50° C. and stirred for 50 hours. Water (10 mL) is added to the mixture, and extracted with ethyl acetate. The combined organic layer is dried over anhydrous MgSO₄, filtered, and concentrated to give a pink oil which is purified by flash chromatography (hexane:ethyl acetate=1:1) to yield the free acid (1-(benzyloxyamino-methyl)-cyclohexanecarboxylic acid) as a white solid (0.134 g, 0.51 mmol, 85%).

A solution of the free acid (1-(benzyloxyamino-methyl)-cyclohexanecarboxylic acid) (0.133 g, 0.504 mmol, 1 equiv.) in dichloromethane (3 mL) is cooled to 0° C. under a blanket of argon. In a separate flask, formic acid (0.19 mL, 5.04 mmol, 10 equiv.) is cooled to 0° C. under a blanket of argon, and acetic anhydride (0.1 mL, 1.01 mmol, 2 equiv.) is added dropwise. The solution is stirred at 0° C. for 15 minutes. The resulting mixed anhydride is slowly transferred via syringe into the original reaction vessel. The mixture is stirred at 0° C. for 30 minutes, then allowed to warm up to rt. Water (10 mL) is added to the mixture, and the aqueous layer extracted with dichloromethane. The combined organic layer is dried over anhydrous MgSO₄, filtered, and concentrated to give a yellow solid which is purified by flash chromatography (hexane:ethyl acetate=1:1) to yield the title compound M-5 as a white solid (0.133 g, 0.46 mmol, 90%).

### General Procedure N: Synthesis of 1-[2-(tert-Butyl-dimethyl-silanyloxy)-ethyl]-2-pyrrolidin-2-yl-1H-benzoimidazole (N-3)

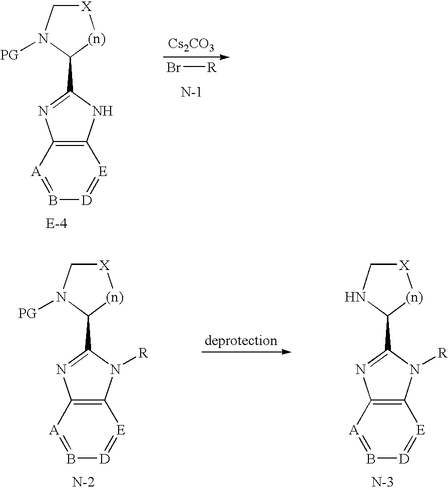

### Step 1: 2-{1-[2-(tert-Butyl-dimethyl-silanyloxy)-ethyl]-1H-benzoimidazol-2-yl}-pyrrolidine-1-carboxylic acid benzyl ester (N-2)

2-(1H-Benzoimidazol-2-yl)-pyrrolidine-1-carboxylic acid benzyl ester E-4 (4.0 mmol) is dissolved in acetonitrile (15 ml), (2-Bromo-ethoxy)-tert-butyl-dimethyl-silane (1.91 g, 8.0 mmol, 2 equiv.) and Cs₂CO₃ (3.9 g, 12.0 mmol, 3 equiv.) are added and the reaction is heated at 60° C. overnight. After cooling to rt the mixture is filtered and the filtrate is concentrated under reduced pressure. The residues are dissolved in ethyl acetate (100 ml) and washed with brine (30 ml). The crude product is purified by flash chromatography (hexane:ethyl acetate=70:30) to yield the title compound N-2 as slightly yellow paste (1.3 g, 2.7 mmol, 67.8%).

### Step 2: 1-[2-(tert-Butyl-dimethyl-silanyloxy)-ethyl]-2-pyrrolidin-2-yl-1H-benzoimidazole (N-3)

2-{1-[2-(tert-Butyl-dimethyl-silanyloxy)-ethyl]-1H-benzoimidazol-2-yl}-pyrrolidine-1-carboxylic acid benzyl ester N-2 (1.3 g, 2.7 mmol) is dissolved in ethyl acetate/MeOH (1:1, mL) and added slowly to 10% Pd/C (130 mg, 0.1 equiv.). Through the mixture is bubbled H₂ gas for 1 hr until LC-MS shows the reaction is completed. Pd/C is filtered off and the filtrate is concentrated under reduced pressure to yield the title compound 1-[2-(tert-Butyl-dimethyl-silanyloxy)-ethyl]-2-pyrrolidin-2-yl-1H-benzoimidazole N-3 as slightly yellow oil (818 mg, 2.4 mmol, 88.9%).

### EXAMPLES

### Example 1

### N-{(R)-2-[(S)-2-(1H-Benzoimidazol-2-yl)-pyrrolidine-1-carbonyl]-hexyl}-N-hydroxy-formamide

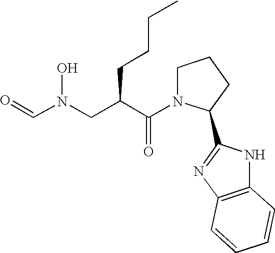

The title compound is prepared from (R)-2-[(Benzyloxy-formyl-amino)-methyl]-hexanoic acid A-7 (preparation is described in General Procedure A) and (S)-2-Pyrrolidin-2-yl-1H-benzoimidazole E-5 (preparation is described in General Procedure E; PG=CBz) according to General Procedure B.

1H-NMR (MeOH-d₄, rotamers): δ 8.28 (s, 0.30H), 7.86-7.84 (d, 0.45H), 7.77 (s, 0.25H), 7.65-7.40 (m, 2H), 7.30-7.12 (m, 2H), 5.48 (s, 1H), 5.42 (d, 0.33H), 5.28-5.15 (m, 0.67H), 3.93-3.78 (m, 1.33H), 3.80-3.50 (m, 1.70H), 3.47-3.35 (m, 0.95H), 3.15-3.00 (m, 0.71H), 2.88-2.74 (m, 0.31H), 2.55-1.90 (m, 4H), 1.65-1.05 (m, 6H), 0.93-0.90 (m, 1.15H), 0.84-0.80 (m, 1.85H).

13C-NMR (MeOH-d₄, rotamers): δ 163.4, 159.9, 159.7, 123.5, 115.2, 57.6, 56.8, 53.4, 53.3, 51.2, 47.7, 44.3, 43.1, 43.0, 34.4, 32.4, 30.9, 30.2, 25.8, 23.8, 23.3, 14.2.

ES-MS: calcd. for C₁₉H₂₆N₄O₃ (358.44); found (pos.): 359.7 [M+H]; found (neg.): 357.4 [M−H].

### Example 2

### N-{(R)-2-[(S)-2-(1H-Benzoimidazol-2-yl)-pyrrolidine-1-carbonyl]-heptyl}-N-hydroxy-formamide

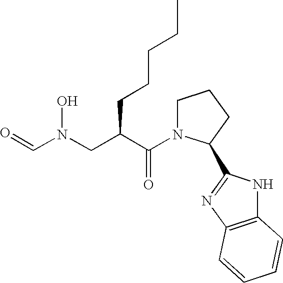

The title compound is prepared from (R)-2-[(Benzyloxy-formyl-amino)-methyl]-heptanoic acid A-7 (R2=n-pentyl, preparation is described in General Procedure A) and (S)-2-Pyrrolidin-2-yl-1H-benzoimidazole E-5 (preparation is described in General Procedure E; PG=CBz) according to General Procedure B.

1H-NMR (MeOH-d₄, rotamers): δ 7.87 (s, 0.64H), 7.84 (s, 0.36H), 7.70-7.90 (m, 0.68H), 7.51-7.49 (m, 1.60H), 7.25-7.18 (m, 1.72H), 5.50-5.30 (m, 1H), 5.22-5.19 (m, 1H), 3.90-3.87 (m, 1.84H), 3.74-3.65 (m, 2.08H), 3.47-3.42 (m, 1.07H), 2.40-2.10 (m, 2.61H), 2.10-1.95 (m, 1H), 1.95-1.75 (m, 0.39H), 1.70-1.05 (m, 8H), 1.05-0.85 (m, 1.13H), 0.85-0.70 (m, 1.87H).

13C-NMR (MeOH-d₄, rotamers): 175.5, 175.1, 164.0, 163.4, 159.9, 159.7, 157.0, 125.9, 124.2, 124.0, 123.5, 115.7, 57.6, 56.7, 53.3, 47.7, 43.0, 34.4, 34.2, 33.1, 33.0, 32.3, 31.5, 31.1, 27.6, 25.8, 23.4, 23.3, 14.3.

ES-MS: calcd. for C₂₀H₂₈N₄O₃ (372.47); found (pos.): 374.0 [M+H]; found (neg.): 371.6 [M−H].

### Example 3

### N-{(R)-3-[(S)-2-(1H-Benzoimidazol-2-yl)-pyrrolidine-1-yl]-2-benzyl-3-oxo-propyl}-N-hydroxy-formamide

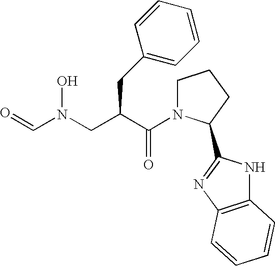

The title compound is prepared from (R)-2-[(Benzyloxy-formyl-amino)-methyl]-3-phenyl-propionic acid A-7 (R2=benzyl, preparation is described in General Procedure A) and (S)-2-Pyrrolidin-2-yl-1H-benzoimidazole E-5 (preparation is described in General Procedure E; PG=CBz) according to General Procedure B.

1H-NMR (MeOH-d₄, rotamers): δ 8.28 (s, 0.15H), 7.96 (s, 0.33H), 7.85 (s, 0.52H), 7.70-7.45 (m, 1.95H), 7.35-7.15 (m, 5.39H), 7.10-6.80 (m, 1.66H), 5.47 (s, 1.75H), 5.15-5.30 (m, 0.25H), 4.34 (t, 0.62H), 4.00 (d, 0.34H), 3.90-3.80 (m, 0.63H), 3.80-3.65 (m, 0.39H), 3.65-3.35 (m, 2.63H), 3.20-3.00 (m, 0.40H), 3.00-2.58 (m, 2.04H), 2.30-2.20 (m, 0.52H), 2.20-2.05 (m, 0.59H), 2.04-1.88 (m, 0.85H), 1.85-1.68 (m, 0.51H), 1.65-1.45 (m, 1.10H), 0.35-0.20 (m, 0.39H).

13C-NMR (MeOH-d₄, rotamers): δ 175.2, 174.8, 174.3, 164.1, 163.5, 160.0, 159.8, 156.1, 155.8, 155.3, 139.6, 139.3, 130.2, 130.0, 129.8, 129.5, 129.3, 128.2, 128.1, 127.6, 127.5, 124.1, 123.9, 123.6, 57.1, 56.3, 54.8, 53.7, 47.3, 47.1, 46.9, 38.8, 38.3, 33.7, 33.4, 23.2, 23.1.

ES-MS: Calcd. for C₂₂H₂₄N₄O₃ (392.46); found (pos.): 394.0 [M+1]; found (neg.): 391.4 [M-1].

### Example 4

### N-{(R)-3-[(S)-2-(1H-Benzoimidazol-2-yl)-pyrrolidin-1-yl]-2-cyclopentylmethyl-3-oxo-propyl}-N-hydroxy-formamide

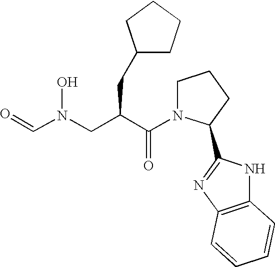

The title compound is prepared from (R)-2-[(Benzyloxy-formyl-amino)-methyl]-3-cyclopentyl-propionic acid A-7 (R2=cyclopentylmethyl, preparation is described in General Procedure A) and (S)-2-Pyrrolidin-2-yl-1H-benzoimidazole E-5 (preparation is described in General Procedure E; PG=CBz) according to General Procedure B.

1H-NMR (MeOH-d₄, rotamers): δ 8.28 (s, 0.17H), 8.20 (s, 0.08H), 7.83 (0.59H), 7.75 (s, 0.16H), 7.63-7.47 (m, 2H), 7.30-7.16 (m, 2H), 5.41 (d, J=6.5, 0.35H), 5.26-5.18 (m, 0.65H), 3.95-3.82 (m, 1.49H), 3.80-3.58 (m, 1.75H), 3.49-3.35 (m, 0.85H), 3.17-3.08 (m, 0.23H), 2.93-2.85 (m, 0.18H), 2.69-2.60 (m, 0.20H), 2.53-2.40 (m, 0.38H), 2.40-2.15 (m, 2.35H), 2.13-1.97 (m, 1.06H), 1.94-1.28 (m, 9.46H), 1.25-0.92 (m, 2.11H).

13C-NMR (MeOH-d₄, rotamers): δ 176.3, 175.7, 175.2, 174.9, 163.9, 163.4, 159.9, 159.7, 156.8, 156.1, 139.2, 124.2, 123.9, 123.5, 115.7, 57.6, 56.7, 53.4, 47.9, 43.9, 43.3, 42.5, 42.4, 38.8, 38.2, 37.6, 37.2, 34.6, 34.4, 34.3, 33.9, 33.8, 32.2, 26.3, 26.1, 25.9, 23.3, 23.2.

ES-MS: calcd. for C₂₁H₂₈N₄O₃ (384.48); found (pos.): 385.7 [M+H]; found (neg.): 383.3 [M−H], 497.5 [M+CF₃CO₂].

### Example 5

### N-{(R)-2-[(S)-2-(1H-Benzoimidazol-2-yl)-pyrrolidine-1-carbonyl]-pentyl}-N-hydroxy-formamide

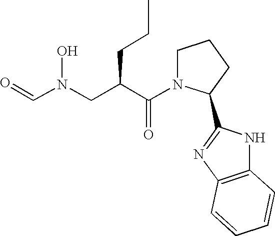

The title compound is prepared from (R)-2-[(Benzyloxy-formyl-amino)-methyl]-pentanoic acid A-7 (R2=propyl, preparation is described in General Procedure A) and (S)-2-Pyrrolidin-2-yl-1H-benzoimidazole E-5 (preparation is described in General Procedure E; PG=CBz) according to General Procedure B.

1H-NMR (MeOH-d₄, rotamers): δ 8.28 (s, 0.16H), 8.20 (s, 0.07H), 7.87 (0.18H), 7.84 (s, 0.39H), 7.77 (s, 0.17H), 7.65-7.47 (m, 1.77H), 7.29-7.16 (m, 1.91H), 5.43 (d, J=7.6, 0.36H), 5.25-5.17 (m, 0.61H), 3.96-3.80 (m, 1.42H), 3.78-3.59 (m, 1.75H), 3.49-3.39 (m, 0.84H), 3.15-3.06 (m, 0.26H), 2.97-2.85 (m, 0.24H), 2.80 (s, 1.13H), 2.72-2.62 (m, 0.22H), 2.53-1.98 (m, 3.65H), 1.94-1.66 (m, 0.39H), 1.64-1.21 (m, 4.05H), 0.97-0.82 (m, 3.0H).

13C-NMR (MeOH-d₄, rotamers): δ 176.3, 175.7, 175.2, 164.0, 163.4, 159.9, 159.7, 157.1, 156.1, 155.7, 139.2, 124.2, 123.9, 123.5, 115.7, 57.6, 56.7, 56.9, 53.2, 47.7, 42.8, 38.9, 34.8, 34.2, 33.2, 32.5, 25.8, 21.3, 21.2, 14.6, 14.4.

ES-MS: calcd. for C₁₈H₂₄N₄O₃ (344.18); found (pos.): 346.1 [M+H]; found (neg.): 343.5 [M−H], 457.7 [M+CF₃CO₂].

### Example 6

### N-{(R)-2-[(2S,4R)-2-(1H-Benzoimidazol-2-yl)-4-fluoro-pyrrolidine-1-carbonyl]-hexyl}-N-hydroxy-formamide

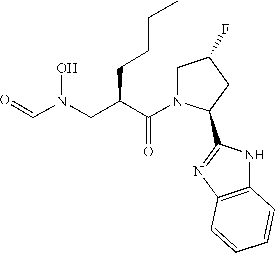

The title compound is prepared from (R)-2-[(Benzyloxy-formyl-amino)-methyl]-hexanoic acid A-7 (R2=n-butyl, preparation is described in General Procedure A) and 2-((2S,4R)-4-Fluoro-pyrrolidin-2-yl)-1H-benzoimidazole E-5 (preparation is described in General Procedure E; PG=CBz) according to General Procedure B.

1H-NMR (MeOH-d₄, rotamers): δ 8.24 (s, 0.27H), 8.11 (s, 0.21H), 7.83 (0.41H), 7.66 (s, 0.11H), 7.63 (s, 0.13H), 7.59-7.48 (m, 2H), 7.28-7.18 (m, 2H), 5.54-5.47 (m, 0.63H), 5.44-5.37 (br.s, 0.50H), 5.29 (t, J=8.5, 0.87H), 4.40-3.98 (m, 1.81H), 3.82-3.64 (m, 1.09H), 3.54-3.44 (m, 0.44H), 3.42-3.40 (m, 0.25H), 3.39-3.35 (m, 0.28H), 3.35-3.32 (m, 0.12H), 3.22-3.13 (m, 0.46H), 3.11-3.03 (m, 0.31H), 3.00-2.85 (m, 0.41H), 2.81-2.67 (m, 0.90H), 2.62-2.52 (m, 0.42H), 2.52-2.36 (m, 0.63H), 1.56-1.28 (m, 3.07H), 1.25-1.08 (m, 2.91H), 0.95-0.87 (m, 0.78H), 0.80-0.71 (m, 2.23H).

13C-NMR (MeOH-d₄, rotamers): δ 175.1, 165.0, 163.0, 160.5, 158.6, 156.2, 139.0, 124.4, 122.9, 116.6, 115.0, 55.9, 54.4, 43.5, 42.2, 31.0, 30.0, 23.7, 14.8, 13.6.

ES-MS: calcd. for C₁₉H₂₅FN₄O₃ (376.19); found (pos.): 377.7 [M+H]; found (neg.): 375.6 [M−H].

### Example 7

### N-{(R)-2-[(S)-2-(1H-Benzoimidazol-2-yl)-4-fluoro-pyrrolidine-1-carbonyl]-heptyl}-N-hydroxy-formamide

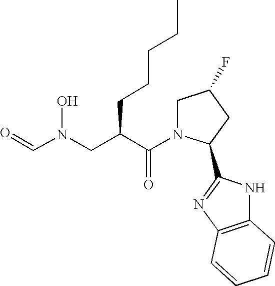

The title compound is prepared from (R)-2-[(Benzyloxy-formyl-amino)-methyl]-heptanoic acid A-7 (R2=n-pentyl, preparation is described in General Procedure A) and 2-((2S,4R)-4-Fluoro-pyrrolidin-2-yl)-1H-benzoimidazole E-5 (preparation is described in General Procedure E; PG=CBz) according to General Procedure B.

1H-NMR (MeOH-d₄, rotamers): δ 8.22 (s, 0.41H), 7.78 (s, 0.59H), 7.70-7.45 (m, 2H), 7.35-7.12 (m, 2H), 5.55-5.44 (m, 1.15H), 5.44-5.32 (m, 0.67H), 5.32-5.23 (m, 1.18H), 1.68-1.05 (m, 8H), 0.95-0.82 (m, 1H), 0.82-0.65 (m, 2H).

ES-MS: calcd. for C₂₀H₂₇FN₄O₃ (390.46); found (pos.): 391.7 [M+H]; found (neg.): 389.5 [M−H].

### Example 8

### N-{(R)-3-[(2S,4R)-2-(1H-Benzoimidazol-2-yl)-4-fluoro-pyrrolidin-1-yl]-2-cyclopentylmethyl-3-oxo-propyl}-N-hydroxy-formamide

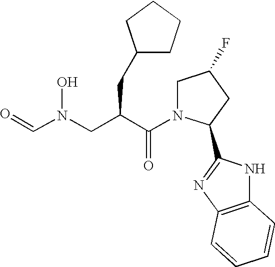

The title compound is prepared from (R)-2-[(Benzyloxy-formyl-amino)-methyl]-3-cyclopentyl-propionic acid A-7 (R2=cyclopentylmethyl, preparation is described in General Procedure A) and 2-((2S,4R)-4-Fluoro-pyrrolidin-2-yl)-1H-benzoimidazole E-5 (preparation is described in General Procedure E; PG=CBz) according to General Procedure B.

1H-NMR (MeOH-d₄, rotamers): δ 8.23 (s, 0.30H), 8.17 (s, 0.06H), 7.83 (0.42H), 7.63 (s, 0.22H), 7.59-7.48 (m, 1.93H), 7.28-7.17 (m, 1.97H), 5.54-5.45 (m, 0.62H), 5.43-5.37 (br.s, 0.51H), 5.31 (t, J=8.5, 8.3, 0.88H), 4.38-4.15 (m, 1.0H), 4.15-3.94 (m, 0.79H), 3.87-3.68 (m, 1.13H), 3.49-3.32 (m, 1.29H), 3.25-3.13 (m, 0.58H), 3.13-3.04 (m, 0.34H), 3.00-2.84 (m, 0.39H), 2.84-2.58 (m, 1.30H), 2.58-2.43 (m, 0.54H), 2.43-2.37 (m, 0.13H), 1.87-1.14 (m, 9.50H), 1.11-0.81 (m, 1.91H).

13C-NMR (MeOH-d₄, rotamers): δ 175.2, 163.0, 160.4, 155.9, 124.4, 122.8, 115.1, 55.8, 54.3, 50.0, 43.0, 41.7, 39.3, 38.1, 34.0, 32.6, 27.3, 26.0, 24.7.

ES-MS: calcd. for C₂₁H₂₇FN₄O₃ (402.21); found (pos.): 403.4 [M+H]; found (neg.): 401.3 [M−H].

### Example 9

### N-Hydroxy-N-{(R)-2-[(S)-2-(3H-imidazo[4,5-c]pyridin-2-yl)-pyrrolidine-1-carbonyl]-hexyl}-formamide

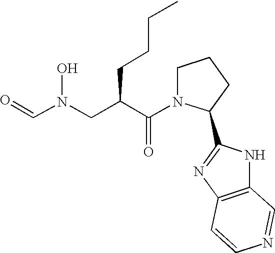

The title compound is prepared from (R)-2-[(Benzyloxy-formyl-amino)-methyl]-hexanoic acid A-7 (R2=n-butyl, preparation is described in General Procedure A) and (S)-2-Pyrrolidin-2-yl-3H-imidazo[4,5-c]pyridine E-5 (preparation is described in General Procedure E; PG=CBz) according to General Procedure B.

1H-NMR (MeOH-d₄, rotamers): δ 8.89 (s, 0.14H), 8.84 (s, 0.11H), 8.79 (s, 0.61H), 8.31-8.25 (d, 0.94H), 8.24-8.2 (d, 0.17H), 7.86-7.8 (m, 0.56H), 7.76 (s, 0.15H), 7.74-7.70 (m, 0.17H), 7.69-7.65 (m, 0.13H), 7.59-7.55 (d, 0.64H), 5.49-5.45 (m, 0.29H), 5.28-5.2 (m, 0.71H), 4.68-4.6 (dd, J=0.04, 0.58H), 8.7-8.6 (m, 1.3H), 3.81-3.63 (m, 1.67H), 3.5-3.4 (m, 0.9H), 3.78-3.7 (m, 0.43H), 2.91-2.83 (m, 0.26H), 2.73-2.65 (m, 0.18H), 2.35-2.51 (m, 1.3H), 2.37-2.25 (m, 0.9), 2.26-2.06 (m, 1.7), 1.64-1.5 (m, 1.2H), 1.52-1.4 (m, 1.1H), 1.42-1.2 (m, 4.8H), 0.96-0.9 (m, 1.1H), 0.9-0.78 (2H).

ES-MS: calcd. for C₁₈H₂₅N₅O₃ (359.4); found (pos.): 360.6 [M+H].

### Example 10

### N-{(R)-2-Cyclopentylmethyl-3-[(S)-2-(3H-imidazo[4,5-c]pyridine-2-yl)-pyrrolidin-1-yl]-3-oxo-propyl}-N-hydroxy-formamide

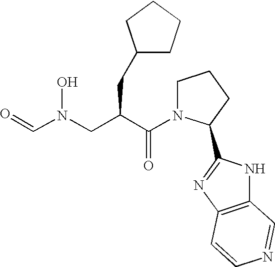

The title compound is prepared from (R)-2-[(Benzyloxy-formyl-amino)-methyl]-3-cyclopentyl-propionic acid A-7 (R2=cyclopentylmethyl, preparation is described in General Procedure A) and (S)-2-Pyrrolidin-2-yl-3H-imidazo[4,5-c]pyridine E-5 (preparation is described in General Procedure E; PG=CBz) according to General Procedure B.

1H-NMR (MeOH-d₄, rotamers): δ 8.38-8.19 (m, 1.57H), 7.83 (s, 0.74H), 7.74 (s, 0.49H), 7.65 (br.s, 0.86H), 5.30-5.22 (m, 1.0H), 3.98-3.83 (m, 2.05H), 3.83-3.58 (m, 2.63H), 3.53-3.41 (m, 1.09H), 3.34 (s, 1.28H), 3.17-3.07 (m, 0.41H), 2.45-2.00 (m, 5.07H), 1.89-1.72 (m, 2.92H), 1.68-1.23 (m, 0.75H), 1.23-0.90 (m, 2.87H).

13C-NMR (MeOH-d₄, rotamers): δ 175.3, 164.0, 159.7, 56.8, 53.5, 42.4, 38.8, 37.3, 33.9, 33.7, 32.3, 26.3, 26.1, 25.9, 23.4.

### Example 11

### N-{(R)-2-Cyclopentylmethyl-3-[(2S,4R)-4-fluoro-2-(3H-imidazo[4,5-c]pyridin-2-yl)-pyrrolidin-1-yl]-3-oxo-propyl}-N-hydroxy-formamide

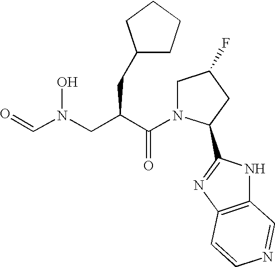

The title compound is prepared from (R)-2-[(Benzyloxy-formyl-amino)-methyl]-3-cyclopentyl-propionic acid A-7 (R2=cyclopentylmethyl, preparation is described in General Procedure A) and 2-((2S,4R)-4-Fluoro-pyrrolidin-2-yl)-3H-imidazo[4,5-c]pyridine E-5 (preparation is described in General Procedure E; PG=CBz) according to General Procedure B.

1H-NMR (MeOH-d₄, rotamers): δ 8.88 (s, 0.08H), 8.85 (s, 0.71H), 8.32 (d, J=5.9, 0.71H), 8.30-8.23 (m, 0.54H), 8.15 (s, 0.74H), 7.82 (s, 0.36H), 7.76-7.63 (m, 1.10H), 5.55 (br.s, 0.62H), 5.42 (br.s, 0.54H), 5.34 (t, J=9.1, 8.5, 0.85H), 4.40-4.19 (m, 1.04H), 4.19-3.97 (m, 0.89H), 3.90-3.78 (m, 0.46H), 3.78-3.66 (m, 0.69H), 3.54-3.38 (m, 0.98H), 3.26-3.16 (m, 0.51H), 3.16-3.06 (m, 0.36H), 2.84-2.67 (m, 0.45H), 2.57-2.44 (m, 0.55H), 1.92-1.70 (m, 1.52H), 1.70-1.24 (m, 7.46H), 1.24-1.15 (m, 0.27H), 1.15-1.00 (m, 1.02H), 1.00-0.83 (m, 0.80H).

13C-NMR (MeOH-d₄, rotamers): δ 175.5, 175.3, 165.5, 164.0, 161.2, 159.6, 159.4, 140.5, 140.4, 138.1, 112.4, 111.0, 94.3, 94.0, 92.5, 92.3, 57.0, 55.2, 42.4, 38.9, 38.6, 37.4, 33.9, 26.0.

ES-MS: calcd. for C₂₀H₂₆FN₅O₃ (403.20); found (pos.): 404.8 [M+H]; found (neg.): 402.4 [M−H].

### Example 12

### N-{(R)-2-[(2S,4R)-4-Fluoro-2-(3H-imidazo[4,5-c]pyridin-2-yl)-pyrrolidine-1-carbonyl]-hexyl}-N-hydroxy-formamide

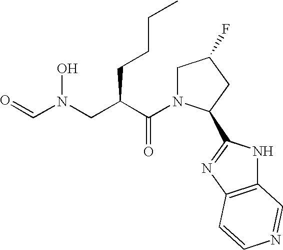

The title compound is prepared from (R)-2-[(Benzyloxy-formyl-amino)-methyl]-hexanoic acid A-7 (R2=n-butyl, preparation is described in General Procedure A) and 2-((2S,4R)-4-Fluoro-pyrrolidin-2-yl)-3H-imidazo[4,5-c]pyridine E-5 (preparation is described in General Procedure E; PG=CBz) according to General Procedure C.

1H-NMR (MeOH-d₄, rotamers): δ 8.86 (s, 0.77H), 8.32 (d, J=5.88, 0.72H), 8.32-8.28 (m, 0.14), 8.28-8.24 (m, 0.41H), 8.19 (s, 0.80H), 7.82 (s, 0.37H), 7.80-7.70 (d, J=6.1, 0.13H), 7.70-7.56 (m, 1.02H), 5.66-5.50 (m, 0.59H), 5.41 (bs, 0.47H), 5.36-5.20 (m, 0.83H), 4.50-3.94 (m, 1.79H), 3.90-3.66 (m, 1.13H), 3.66-3.44 (m, 0.55H), 3.44-3.34 (m, 0.53H), 3.24-3.14 (m, 0.46), 3.14-3.04 (m, 0.32H), 3.04-2.84 (m, 0.41H), 2.84-2.66 (m, 0.86H), 2.64-2.52 (m, 0.49H), 2.52-2.36 (m, 0.49H), 1.60-1.04 (m, 6.84H), 1.0-0.84 (m, 0.83H), 0.84-0.70 (m, 2.16H).

ES-MS: calcd. for C₁₈H₂₄FN₅O₃ (377.42); found (pos.): 378.9 [M+H].

### Example 13

### N-{(R)-2-[(S)-2-(6,7-Dihydro-1H-5,8-dioxa-1,3-diaza-cyclopenta[b]naphthalen-2-yl)-pyrrolidine-1-carbonyl]-hexyl}-N-hydroxy-formamide

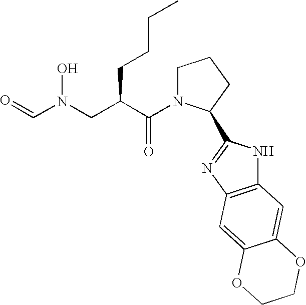

The title compound is prepared from (R)-2-[(Benzyloxy-formyl-amino)-methyl]-hexanoic acid A-7 (R2=n-butyl, preparation is described in General Procedure A) and (S)-2-Pyrrolidin-2-yl-6,7-dihydro-1H-5,8-dioxa-1,3-diaza-cyclopenta[b]naphthalene E-5 (preparation is described in General Procedure E; PG=CBz) according to General Procedure B.

1H-NMR (MeOH-d₄, rotamers): δ 8.27 (s, 0.14H), 8.23 (s, 0.14H), 7.89 (s, 0.23H), 7.83 (s, 0.39H), 7.77 (s, 0.15H), 7.04 (s, 0.30H), 6.97 (s, 0.37H), 6.93 (s, 0.98H), 5.34 (d, J=7.5, 0.41H), 5.19-5.11 (m, 0.59H), 3.92-3.79 (m, 1.41H), 3.77-3.55 (m, 1.87H), 3.48-3.38 (m, 0.88H), 3.25-3.17 (m, 0.45H), 3.12-3.02 (m, 0.22H), 2.67-2.59 (m, 0.23H), 2.44-2.10 (m, 2.67H), 2.10-1.96 (m, 1.08H), 1.89-1.77 (m, 0.42H), 1.63-1.16 (m, 6.74H), 0.97-0.79 (m, 3.33H).

13C-NMR (MeOH-d₄, rotamers): δ 176.2, 175.6, 175.1, 164.0, 163.4, 159.9, 159.7, 156.5, 155.6, 155.1, 142.9, 142.8, 142.5, 102.5, 65.5, 56.7, 53.3, 42.9, 32.2, 30.9, 30.2, 30.1, 25.8, 24.0, 23.9, 23.8, 14.2, 14.2.

ES-MS: calcd. for C₂₁H₂₈N₄O₅ (416.21); found (pos.): 417.0 [M+H]; found (neg.): 415.5 [M−H].

### Example 14

### N-{(R)-2-[(S)-2-(7-Chloro-5-trifluoromethyl-1H-benzoimidazol-2-yl)-pyrrolidine-1-carbonyl]-hexyl}-N-hydroxy-formamide

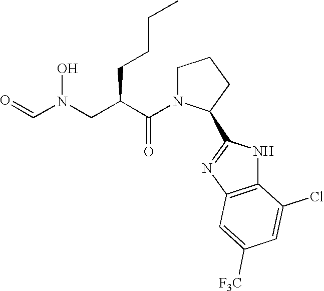

The title compound is prepared from (R)-2-[(Benzyloxy-formyl-amino)-methyl]-hexanoic acid A-7 (R2=n-butyl, preparation is described in General Procedure A) and 7-Chloro-2-(S)-pyrrolidin-2-yl-5-trifluoromethyl-1H-benzoimidazole E-5 (preparation is described in General Procedure E; PG=CBz) according to General Procedure B.

1H-NMR (MeOH-d₄, rotamers): δ 8.54 (s, 0.03H), 8.28 (s, 0.22H), 7.89-7.71 (m, 1.59H), 7.57-7.47 (m, 0.80H), 7.42 (s, 0.06H), 7.32 (s, 0.06H), 5.46-5.39 (d, 0.23H), 5.25-5.17 (m, 0.77H), 3.97-3.75 (m, 2.03H), 3.75-3.55 (m, 1.71H), 3.49-3.33 (m, 1.11H), 3.14-3.08 (m, 0.40H), 2.47-1.88 (m, 4.52H), 1.65-1.18 (m, 7.15H), 1.01-0.77 (m, 3.29H).

13C-NMR (MeOH-d₄, rotamers): δ 175.1, 164.0, 161.0, 159.7, 159.7, 126.5, 126.2, 119.8, 56.8, 53.3, 43.1, 43.0, 32.6, 31.0, 30.1, 25.9, 24.0, 23.8, 14.2.

ES-MS: calcd. for C₂₀H₂₄ClF₃N₄O₃ (460.15); found (pos.): 461.2 [M+H]; found (neg.): 459.6 [M−H].

### Example 15

### N-Hydroxy-N—((R)-2-{(S)-2-[5-(4-methyl-piperazin-1-yl)-1H-benzoimidazol-2-yl]-pyrrolidine-1-carbonyl}-hexyl)-formamide

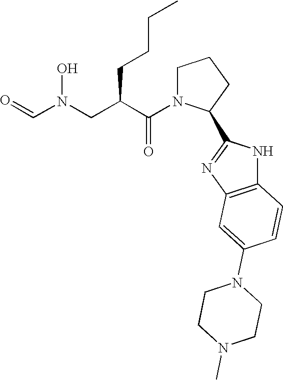

The title compound is prepared from (R)-2-[(Benzyloxy-formyl-amino)-methyl]-hexanoic acid A-7 (R2=n-butyl, preparation is described in General Procedure A) and 5-(4-Methyl-piperazin-1-yl)-2-(S)-pyrrolidin-2-yl-1H-benzoimidazole E-5 (preparation is described in General Procedure E; PG=CBz) according to General Procedure B.

1H-NMR (MeOH-d₄, rotamers): δ 8.35 (s, 0.86H), 8.22 (s, 0.14H), 7.85-7.70 (m, 0.65H), 7.50-7.35 (m, 0.84H), 7.13-6.90 (m, 1.52H), 5.40-5.30 (m, 0.40H), 5.20-5.08 (m, 0.60H), 3.90-3.79 (m, 1.77H), 3.79-3.60 (m, 2.39H), 3.50-3.38 (m, 1.20H), 3.25-3.07 (m, 5.67H), 2.90-2.68 (m, 3.63H), 2.48-2.17 (m, 3.42H), 2.10-1.98 (m, 1.41H), 1.90-1.73 (m, 0.54H), 1.60-1.18 (m, 6H), 0.98-0.86 (m, 1.24H), 0.86-0.72 (m, 1.76H).

13C-NMR (MeOH-d₄, rotamers): δ 175.8, 175.1, 168.5, 164.0, 163.3, 160.0, 159.7, 156.9, 148.3, 116.9, 116.8, 116.5, 116.4, 103.5, 55.4, 55.3, 44.4, 43.0, 32.3, 30.9, 30.2, 25.8, 23.8, 14.3, 14.2.

ES-MS: calcd. for C₂₄H₃₆N₆O₃ (456.59); found (pos.): 457.6 [M+H]; found (neg.): 455.5 [M−H].

### Example 16

### N-{(R)-2-[(S)-2-(5-Fluoro-1H-Benzoimidazol-2-yl)-pyrrolidine-1-carbonyl]-hexyl}-N-hydroxy-formamide

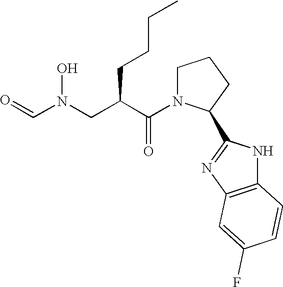

The title compound is prepared from (R)-2-[(Benzyloxy-formyl-amino)-methyl]-hexanoic acid A-7 (R2=n-butyl, preparation is described in General Procedure A) and 5-Fluoro-2-(S)-pyrrolidin-2-yl-1H-benzoimidazole E-5 (preparation is described in General Procedure E; PG=CBz) according to General Procedure B.

1H-NMR (MeOH-d₄, rotamers): 8.28 (s, 0.18H), 8.10 (s, 0.17H), 7.90-7.70 (m, 0.65H), 7.53-7.32 (m, 1H), 7.20-7.15 (m, 1H), 6.98-6.90 (m, 1H), 5.45-5.30 (m, 0.34H), 5.20-5.07 (m, 0.66H), 3.90-3.78 (m, 1.46H), 3.78-3.53 (m, 1.86H), 3.50-3.35 (m, 0.87H), 3.15-3.00 (m, 0.29H), 2.95-2.80 (m, 0.23H), 2.57-1.78 (m, 4.29H), 1.68-1.10 (m, 6H), 1.00-0.75 (m, 3H).

13C-NMR (MeOH-d₄, rotamers): δ 175.1, 162.1, 158.7, 112.4, 110.8, 110.5, 57.5, 56.1, 53.3, 51.9, 47.4, 43.6, 32.3, 30.9, 30.2, 27.1, 25.8, 23.8, 23.3, 16.1, 14.9, 13.6.

ES-MS: calcd. for C₁₉H₂₅FN₄O₃ (376.43); found (pos.): 377.8 [M+H]; found (neg.): 375.2 [M−H], 489.2 [M+CF₃CO₂].

### Example 17

### N-{(R)-2-[(S)-2-(5-Chloro-1H-benzoimidazol-2-yl)-pyrrolidine-1-carbonyl]-hexyl}-N-hydroxy-formamide

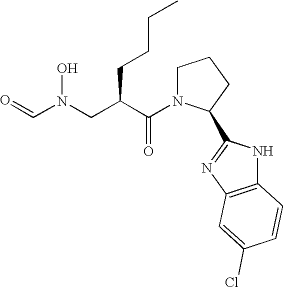

The title compound is prepared from (R)-2-[(Benzyloxy-formyl-amino)-methyl]-hexanoic acid A-7 (R2=n-butyl, preparation is described in General Procedure A) and 5-Chloro-2-(S)-pyrrolidin-2-yl-1H-benzoimidazole E-5 (preparation is described in General Procedure E; PG=Boc) according to General Procedure C.

1H-NMR (MeOH-d₄, rotamers): δ 8.23 (s, 0.30H), 8.17 (s, 0.06H), 7.83 (0.42H), 7.63 (s, 0.22H), 7.59-7.48 (m, 1.93H), 7.28-7.17 (m, 1.97H), 5.54-5.45 (m, 0.62H), 5.43-5.37 (br.s, 0.51H), 5.31 (t, J=8.5, 8.3, 0.88H), 4.38-4.15 (m, 1.0H), 4.15-3.94 (m, 0.79H), 3.87-3.68 (m, 1.13H), 3.49-3.32 (m, 1.29H), 3.25-3.13 (m, 0.58H), 3.13-3.04 (m, 0.34H), 3.00-2.84 (m, 0.39H), 2.84-2.58 (m, 1.30H), 2.58-2.43 (m, 0.54H), 2.43-2.37 (m, 0.13H), 1.87-1.14 (m, 9.50H), 1.11-0.81 (m, 1.91H).

13C-NMR (MeOH-d₄, rotamers): δ 175.2, 163.0, 160.4, 155.9, 124.4, 122.8, 115.1, 55.8, 54.3, 50.0, 43.0, 41.7, 39.3, 38.1, 34.0, 32.6, 27.3, 26.0, 24.7.

ES-MS: calcd. for C₁₉H₂₅ClN₄O₃ (392.16); found (pos.): 394.0 [M+H]; found (neg.): 391.3 [M−H]; 505.5 [M+CF₃CO₂].

### Example 18

### N-{(R)-2-[(S)-2-(5,6-Difluoro-1H-Benzoimidazol-2-yl)-pyrrolidine-1-carbonyl]-hexyl}-N-hydroxy-formamide

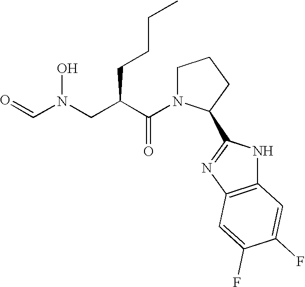

The title compound is prepared from (R)-2-[(Benzyloxy-formyl-amino)-methyl]-hexanoic acid A-7 (R2=n-butyl, preparation is described in General Procedure A) and 5,6-Difluoro-2-(S)-pyrrolidin-2-yl-1H-benzoimidazole E-5 (preparation is described in General Procedure E; PG=CBz) according to General Procedure B.

1H-NMR (MeOH-d₄, rotamers): δ 7.88-7.78 (m, 0.46H), 7.70 (s, 0.13H), 7.52-7.30 (m, 1.41H), 5.48 (s, 0.27H), 5.40-5.35 (m, 0.21H), 5.28-5.08 (m, 0.52H), 3.92-3.77 (m, 1.57H), 3.77-3.60 (m, 1.73H), 3.57-3.35 (m, 0.93H), 3.20-3.04 (m, 0.38H), 2.90-2.78 (m, 0.22H), 2.70-2.58 (m, 0.16H), 2.50-1.95 (m, 4H), 1.65-1.10 (m, 6H), 0.95-0.72 (m, 3H).

ES-MS: calcd. for C₁₉H₂₄F₂N₄O₃ (394.42); found (pos.): 395.5 [M+H]; found (neg.): 393.5 [M−H].

### Example 19

### N-Hydroxy-N-{(R)-2-[(S)-2-(5-methoxy-1H-benzoimidazol-2-yl)-pyrrolidine-1-carbonyl]-hexyl}-formamide

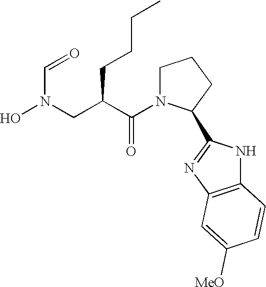

The title compound is prepared from (R)-2-[(Benzyloxy-formyl-amino)-methyl]-hexanoic acid A-7 (R2=n-butyl, preparation is described in General Procedure A) and 5-Methoxy-2-(S)-pyrrolidin-2-yl-1H-benzoimidazole E-5 (preparation is described in General Procedure E; PG=CBz) according to General Procedure B.

1H-NMR (MeOH-d₄, rotamers): δ 8.28 (s, 0.17H), 7.89 (s, 0.17H), 7.84 (s, 0.36H), 7.78 (s, 0.17H), 7.51-7.35 (m, 1H), 7.09-7.0 (m, 0.79H), 6.93-6.8 (m, 1H), 5.45-5.38 (m, 0.4H), 3.94-3.85 (m, 1.24H), 3.86-3.79 (m, 3.3H), 3.8-3.6 (m, 2H), 3.53-3.4 (m, 0.88H), 3.29-3.2 (m, 0.45H), 3.16-3.05 (m, 0.23H), 2.94-2.85 (m, 0.22H), 2.69-2.6 (m, 0.22H), 2.51-2.15 (m, 2.8H), 2.14-2.0 (m, 1H), 1.95 (m, 0.03H), 1.92-1.78 (m, 0.44H), 1.67-1.15 (m, 7H), 0.97-0.88 (m, 1.6H), 0.86-0.75 (m, 1.9H).

ES-MS: calcd. for C₂₀H₂₈N₄O₄ (388.47); found (pos.): 389.7[M+H].

### Example 20

### 2-((S)-1-{(R)-2-[(Formyl-hydroxy-amino)-methyl]-hexanoyl}-pyrrolidin-2-yl)-1H-benzoimidazole-5-carboxylic acid methyl ester

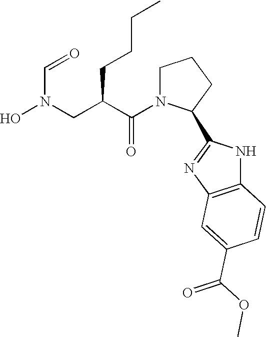

The title compound is prepared from (R)-2-[(Benzyloxy-formyl-amino)-methyl]-hexanoic acid A-7 (R2=n-butyl, preparation is described in General Procedure A) and (S)-2-Pyrrolidin-2-yl-1H-benzoimidazole-5-carboxylic acid methyl ester E-5 (preparation is described in General Procedure E; PG=CBz) according to General Procedure B.

1H-NMR (MeOH-d₄, rotamers): δ 8.29-8.2 (m, 0.8H), 7.99-7.9 (m, 0.7H), 7.85-7.8 (m, 0.2H), 7.76 (s, 0.1H), 7.61-7.52 (m, 0.6H), 5.45-5.4 (m, 0.2H), 5.32-5.25 (m, 0.4H), 5.22-5.15 (m, 0.7H), 4.57-4.55 (m, 0.2H), 3.91 (s, 2.8H), 3.82-3.6 (m, 1H), 3.51-3.4 (m, 0.6H), 3.2-3.02 (m, 0.2H), (s, 0.1H), 2.93-2.8 (m, 0.4H), 2.8 (s, 0.12H), 3.63-3.5 (m, 0.77H), 2.31-2.2 (m, 0.7H), 2.27-2.05 (m, 1.4H), 1.98 (s, 0.3H), 1.74-1.15 (m, 5.2H), 0.99-0.9 (m, 0.8H), 0.94-0.8 (m, 1.4).

ES-MS: calcd. for C₂₁H₂₈N₄O₅ (416.48); found (pos.): 417.5 [M+H].

### Example 21

### N-{(R)-2-[(S)-2-(5-Chloro-6-fluoro-1H-benzoimidazol-2-yl)-pyrrolidine-1-carbonyl]-hexyl}-N-hydroxy-formamide

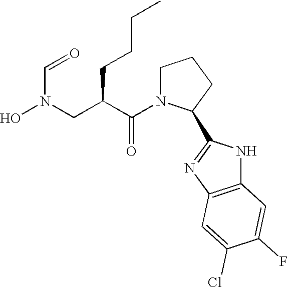

The title compound is prepared from (R)-2-[(Benzyloxy-formyl-amino)-methyl]-hexanoic acid A-7 (R2=n-butyl, preparation is described in General Procedure A) and 5-Chloro-6-fluoro-2-(S)-pyrrolidin-2-yl-1H-benzoimidazole E-5 (preparation is described in General Procedure E; PG=Boc) according to General Procedure C.

1H-NMR (MeOH-d₄, rotamers): δ 8.45 (s, 0.17H), 8.27 (s, 0.18H), 7.83-7.8 (m, 0.51H), 7.77 (s, 0.16H), 7.61-7.55 (m, 0.45H), 7.35-7.3 (m, 0.52H), 5.4-5.35 (m, 0.31H), 5.24-5.15 (m, 0.69H), 3.89-3.8 (m, 1.4H), 3.77-3.6 (m, 1.9H), 3.47-3.35 (m, 1H), 3.27-3.2 (m, 0.66H), 3.14-3.05 (m, 0.42H), 3.0 (s, 0.07H), 2.9-2.85 (m, 0.24H), 2.49-2.4 (m, 0.42H), 2.39-2.2 (m, 1.8H), 2.19-2.0 (m, 1.9H), 1.62-1.5 (m, 1H), 1.49-1.4 (m, 1H), 1.4-1.2 (m, 4.7H), 0.97-0.9 (m, 1H), 0.87-0.8 (m, 1.7H).

ES-MS: calcd. for C₁₉H₂₄ClFN₄O₃ (410.88); found (pos.): 411.5[M+H].

### Example 22

### N-{(R)-2-[(S)-2-(5-Amino-1H-benzoimidazol-2-yl)-pyrrolidine-1-carbonyl]-hexyl}-N-hydroxy-formamide

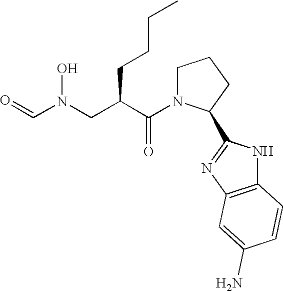

The title compound is prepared from (R)-2-[(Benzyloxy-formyl-amino)-methyl]-hexanoic acid A-7 (R2=n-butyl, preparation is described in General Procedure A) and 5-amino-2-(S)-pyrrolidin-2-yl-1H-benzoimidazole E-5 (preparation is described in General Procedure E; PG=X) according to General Procedure C.

1H-NMR (MeOH-d₄, rotamers): δ 8.00-7.64 (m, 0.85H), 7.34-7.2 (m, 0.77H), 6.85 (s, 7.12H), 6.80-6.65 (m, 0.95H), 5.40-5.30 (m, 0.42H), 5.24-5.05 (m, 0.58H), 3.97-3.77 (m, 1.34H), 3.77-3.54 (m, 1.86H), 3.54-3.33 (m, 0.93H), 3.15-3.0 (m, 0.39H), 3.0-2.84 (m, 0.30H), 2.67-2.54 (m, 0.35H), 2.47-2.10 (m, 2.75H), 2.10-2.02 (m, 1H), 1.67-1.17 (m, 7.28H), 0.97-0.85 (m, 1.29H), 0.85-0.70 (m, 1.57H).

ES-MS: calcd. for C₁₉H₂₇N₅O₃ (373.46); found (pos.): 374.7 [M+H].

### Example 23

### N-Hydroxy-N-{(R)-2-[(S)-2-(5-methyl-1H-benzoimidazol-2-yl)-pyrrolidine-1-carbonyl]-hexyl}-formamide

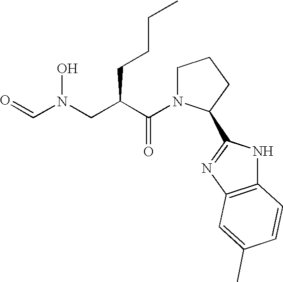

The title compound is prepared from (R)-2-[(Benzyloxy-formyl-amino)-methyl]-hexanoic acid A-7 (R2=n-butyl, preparation is described in General Procedure A) and 5-Methyl-2-(S)-pyrrolidin-2-yl-1H-benzoimidazole E-5 (preparation is described in General Procedure E; PG=CBz) according to General Procedure B.

1H-NMR (MeOH-d₄, rotamers): δ 8.03 (s, 0.19H), 7.87 (s, 0.40H), 7.83 (s, 0.17H), 7.70-7.60 (d, J=8.19, 0.18H), 7.46-7.30 (m, 1.18H), 7.29 (s, 0.74H), 7.26-7.16 (m, 0.34H), 7.16-6.94 (m, 1.05H), 5.46-5.30 (m, 0.39H), 5.26-5.12 (m, 0.61H), 3.96-3.78 (m, 1.45H), 3.78-3.55 (m, 2.08H), 3.50-3.36 (m, 0.91H), 3.28-3.15 (m, 0.70H), 2.42 (s, 3.44H), 2.40-2.14 (m, 2.72H), 2.14-1.95 (m, 1.4H), 1.65-1.48 (m, 1.39H), 1.48-1.10 (m, 7.43H), 0.95-0.85 (m, 1.52H), 0.85-0.75 (m, 1.98H). ES-MS: calcd. for C₂₀H₂₈N₄O₃ (372.47); found (pos.): 373.6 [M+H].

### Example 24

### N-Hydroxy-N-{2-[2-(5-Trifluoromethyl-1H-Benzoimidazol-2-yl)-pyrrolidine-1-carbonyl]-hexyl}-formamide

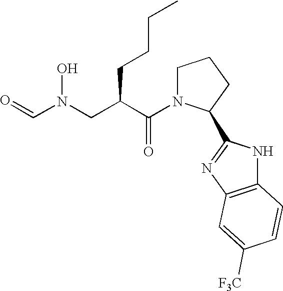

The title compound is prepared from (R)-2-[(Benzyloxy-formyl-amino)-methyl]-hexanoic acid A-7 (R2=n-butyl, preparation is described in General Procedure A) and 2-(S)-Pyrrolidin-2-yl-5-trifluoromethyl-1H-benzoimidazole E-5 (preparation is described in General Procedure E; PG=Boc) according to General Procedure C.

1H-NMR (MeOH-d₄, rotamers): δ 8.27 (s, 0.20H), 7.90-7.60 (m, 2.40H), 7.60-7.40 (m, 0.93H), 5.55-5.40 (m, 0.37H), 5.30-5.13 (m, 0.63H), 3.90-3.55 (m, 3.31H), 3.55-3.33 (m, 0.93H), 3.17-3.0 (m, 0.29H), 3.0-2.85 (m, 0.23H), 2.80 ((s, 2.70H), 2.73-2.55 (m, 0.18H), 2.55-2.42 (m, 0.28H), 2.42-2.33 (m, 0.98H), 2.33-2.23 (m, 0.89H), 2.23-2.13 (m, 0.93H), 2.13-2.0 (m, 1.27H), 1.70-1.03 (m, 7.90H), 1.0-0.83 (m, 1.31H), 0.83-0.70 (m, 1.89H).

ES-MS: calcd. for C₂₀H₂₅F₃N₄O₃ (426.44); found (pos.): 427.6 [M+H].

### Example 25

### Pyrazine-2-carboxylic acid [2-((S)-1-{(R)-2-[(formyl-hydroxy-amino)-methyl]-hexanoyl}-pyrrolidin-2-yl)-1H-benzoimidazol-5-yl]-amide

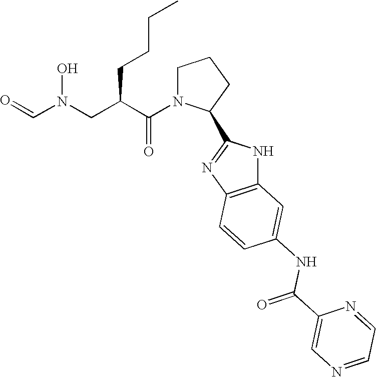

The title compound is prepared from (R)-2-[(Benzyloxy-formyl-amino)-methyl]-hexanoic acid A-7 (R2=n-butyl, preparation is described in General Procedure A) and Pyrazine-2-carboxylic acid ((S)-2-pyrrolidin-2-yl-3H-benzoimidazol-5-yl)-amide E-5 (preparation is described in General Procedure E; PG=CBz) according to General Procedure B.

1H-NMR (MeOH-d₄, rotamers): δ 9.34 (s, 1.19H), 8.81 (s, 1.21H), 8.74 (s, 1.24H), 8.34-8.14 (m, 1.22H), 7.84 (s, 0.53H), 7.80-7.70 (m, 0.30H), 7.70-7.60 (m, 0.18H), 7.60-7.40 (m, 1.87H), 5.54-5.34 (m, 0.35H), 5.34-5.10 (m, 0.65H), 4.0-3.80 (m, 1.58H), 3.80-3.55 (m, 2.17H), 3.55-3.36 (m, 0.93H), 3.17-3.04 (m, 0.31H), 2.98-2.84 (m, 0.20H), 2.76-2.60 (m, 0.21H), 2.54-1.94 (m, 4.63H), 1.70-1.10 (m, 7.74H), 1.0-0.64 (m, 3.63H).

ES-MS: calcd. for C₂₄H₂₉N₇O₄ (479.54); found (pos.): 480.5 [M+H].

### Example 26

### N-[2-((S)-1-{(R)-2-[(Formyl-hydroxy-amino)-methyl]-hexanoyl}-pyrrolidin-2-yl)-3H-benzoimidazol-5-yl]-benzenesulfonamide

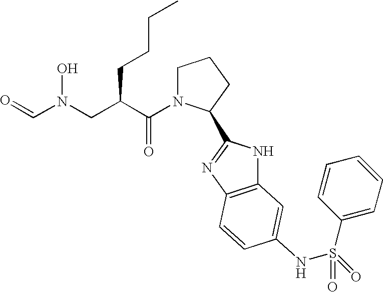

The title compound is prepared from (R)-2-[(Benzyloxy-formyl-amino)-methyl]-hexanoic acid A-7 (R2=n-butyl, preparation is described in General Procedure A) and N—((S)-2-Pyrrolidin-2-yl-3H-benzoimidazol-5-yl)-benzenesulfonamide E-5 (preparation is described in General Procedure E; PG=CBz) according to General Procedure B.

1H-NMR (MeOH-d₄, rotamers): δ 8.36 (s, 0.17H), 7.82 (s, 0.43H), 7.76-7.60 (m, 1.94H), 7.46-7.14 (m, 3.49H), 7.04-6.80 (m, 0.91H), 5.48-5.30 (m, 0.30H), 5.24-5.0 (m, 0.70H), 3.96-3.74 (m, 1.66H), 3.74-3.52 (m, 1.86H), 3.52-3.34 (m, 0.99H), 3.17-3.04 (m, 0.31H), 2.98-2.84 (m, 0.20H), 2.76-2.60 (m, 0.21H), 2.50-1.94 (m, 4.13H), 1.94-1.66 (m, 0.49H), 1.66-1.06 (m, 6.87H), 1.0-0.84 (m, 1.09H), 0.84-0.64 (m, 1.90H).

ES-MS: calcd. for C₂₅H₃₁N₅O₅S (513.62); found (pos.): 514.3 [M+H].

### Example 27

### N-{(R)-2-[(S)-2-(2,2-difluoro-5H-[1,3]dioxolo[4′,5′:4,5]benzo[1,2-d]imidazol-6-yl)-pyrrolidine-1-carbonyl]-hexyl}-N-hydroxy-formamide

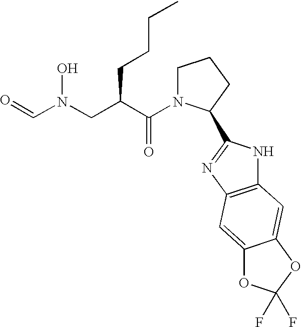

The title compound is prepared from (R)-2-[(Benzyloxy-formyl-amino)-methyl]-hexanoic acid A-7 (preparation is described in General Procedure A) and 2,2-Difluoro-6-(S)-pyrrolidin-2-yl-5H-[1,3]dioxolo[4′,5′:4,5]benzo[1,2-d]imidazole E-5 (preparation is described in General Procedure E, PG=CBz) according to General Procedure B.

1H-NMR (MeOH-d₄, rotamers): δ 8.27 (s, 0.15H), 7.93-7.80 (m, 0.50H), 7.77 (s, 0.17H), 7.50-7.34 (m, 0.45H), 7.31 (s, 1.04H), 5.44-5.30 (m, 0.31H), 5.26-5.03 (m, 0.68H), 3.96-3.74 (m, 1.72H), 3.74-3.54 (m, 1.80H), 3.50-3.33 (m, 0.94H), 2.50-2.13 (m, 3.03H), 2.13-2.03 (m, 0.96H), 1.66-1.50 (m, 1.38H), 1.50-1.34 (m, 1.24H), 1.34-1.14 (m, 5.56H), 1.0-0.86 (m, 1.21H), 0.86-0.73 (m, 1.88H).

ES-MS: calcd. for C₂₀H₂₄F₂N₄O₅ (438.43); found (pos.): 439.6 [M+H].

### Example 28

### N-Hydroxy-N—((R)-2-{(S)-2-[6-(morpholine-4-sulfonyl)-1H-benzoimidazol-2-yl]-pyrrolidine-1-carbonyl}-hexyl)-formamide

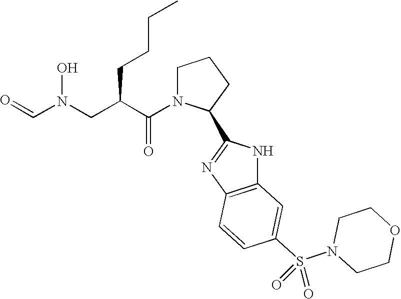

The title compound is prepared from (R)-2-[(Benzyloxy-formyl-amino)-methyl]-hexanoic acid A-7 (preparation is described in General Procedure A) and 6-(Morpholine-4-sulfonyl)-2-(S)-pyrrolidin-2-yl-1H-benzoimidazole E-5 (preparation is described in General Procedure E, PG=CBz) according to General Procedure B.

1H-NMR (MeOH-d₄, rotamers): δ 8.27 (s, 0.21H), 7.95 (s, 0.68H), 7.83 (s, 0.49), 7.75-7.50 (m, 2.13H), 5.64-5.34 (m, 0.25H), 5.30-5.10 (m, 0.75H), 3.90 (t, J=6.63, 1.51H), 3.80-3.60 (m, 5.72H), 3.55-3.34 (m, 0.83H), 3.16-3.03 (m, 0.31H), 3.00-2.84 (m, 3.83H), 2.50-2.24 (m, 1.80H), 2.24-2.00 (m, 1.87H), 1.66-1.50 (m, 1.17H), 1.50-1.35 (m, 1.12H), 1.35-1.10 (m, 4.93H), 1.00-0.85 (m, 0.77H), 0.854-0.70 (m, 2.17H).

ES-MS: calcd. for C₂₃H₃₃N₅O₆S (507.61); found (pos.): 508.6 [M+H].

### Example 29

### N-{(R)-2-[(S)-2-(1,7-Dihydro-imidazo[4,5-f]indazol-6-yl)-pyrrolidine-1-carbonyl]-hexyl}-N-hydroxy-formamide

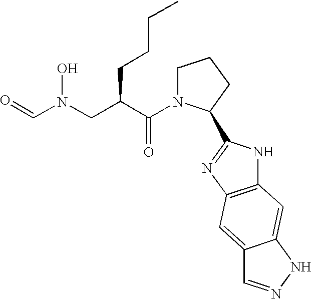

The title compound is prepared from (R)-2-[(Benzyloxy-formyl-amino)-methyl]-hexanoic acid A-7 (preparation is described in General Procedure A) and (S)-6-Pyrrolidin-2-yl-1,7-dihydro-imidazo[4,5-f]indazole E-5 (preparation is described in General Procedure E, PG=CBz) according to General Procedure B.

1H-NMR (MeOH-d₄, rotamers): δ 8.18-8.02 (m, 0.84H), 7.98-7.80 (m, 1.37H), 7.65-7.40 (m, 0.79H), 5.50-5.389 m, 0.36H), 5.28-5.10 (m, 0.64H), 3.95-3.60 (m, 4.76H), 3.55-3.38 (m, 1.24H), 2.50-2.00 (m, 3.58H), 2.00-1.80 (m, 0.42H), 1.68-1.18 (m, 6H), 1.00-0.80 (m, 3H).

ES-MS: calcd. for C₂₀H₂₆N₆O₃ (398.47); found (pos.): 399.1 [M+H]; found (neg.): 397.4 [M−H], 511.4 [M+CF₃CO₂].

### Example 30

### N-Hydroxy-N-{(R)-2-[(S)-2-(9H-purin-8-yl)-pyrrolidine-1-carbonyl]-hexyl}-formamide

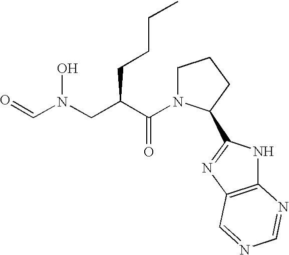

The title compound is prepared from (R)-2-[(Benzyloxy-formyl-amino)-methyl]-hexanoic acid A-7 (preparation is described in General Procedure A) and (S)-8-Pyrrolidin-2-yl-9H-purine E-5 (preparation is described in General Procedure E, PG=CBz) according to General Procedure B.

1H-NMR (MeOH-d₄, rotamers): δ 9.00-8.95 (m, 0.19H), 8.95-8.70 (m, 1.96H), 8.28 (s, 0.23H), 7.89-7.78 (m, 0.62H), 5.50-5.38 (m, 0.18H), 5.30-5.10 (m, 0.82H), 3.95-3.78 (m, 1.39H), 3.78-3.50 (m, 1.87H), 3.50-3.35 (m, 0.74H), 3.18-3.05 (m, 0.26H), 2.90-2.78 (m, 0.16H), 2.60-1.80 (m, 4.58H), 1.70-1.15 (m, 6H), 1.00-0.70 (m, 3H).

### Example 31

### N-{(R)-2-[(S)-2-(6-Cyano-1H-benzoimidazol-2-yl)-pyrrolidine-1-carbonyl]-hexyl}-N-hydroxy-formamide

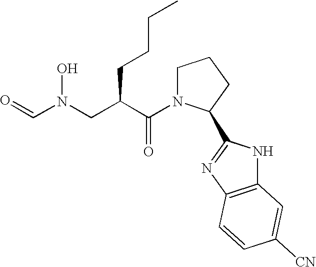

The title compound is prepared from (R)-2-[(Benzyloxy-formyl-amino)-methyl]-hexanoic acid A-7 (preparation is described in General Procedure A) and (S)-2-Pyrrolidin-2-yl-3H-benzoimidazole-5-carbonitrile E-5 (preparation is described in General Procedure E, PG=Boc) according to General Procedure B.

1H-NMR (MeOH-d₄, rotamers): δ 7.90-7.45 (m, 3H), 5.42 (s, 1H), 3.76-3.64 (m, 3H), 3.28-3.18 (m, 2H), 2.75-2.58 (m, 4H), 1.45-1.20 (m, 6H), 0.98-0.72 (m, 3H).

ES-MS: Calcd. for C₂₀H₂₅N₅O₃ (383.45); found (pos.): 384.5 [M+H]; found (neg.): 382.4 [M−H], 496.2 [M+CF₃CO₂].

### Example 32

### N-Hydroxy-N—((R)-2-{(S)-2-[1-(2-methoxy-ethyl)-1H-benzoimidazol-2-yl]-pyrrolidine-1-carbonyl}-hexyl)-formamide

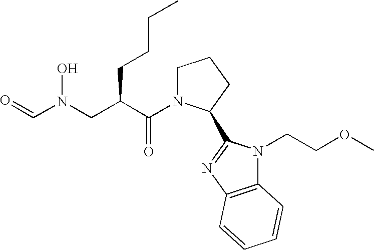

The title compound is prepared from (R)-2-[(Benzyloxy-formyl-amino)-methyl]-hexanoic acid A-7 (preparation is described in General Procedure A) and 1-(2-Methoxy-ethyl)-2-(S)-pyrrolidin-2-yl-1H-benzoimidazole N-3 (preparation is described in General Procedure N, PG=CBz) according to General Procedure B.

1H-NMR (CDCl₃, rotamers): δ 11.4-11.2 (br, 0.5H), 8.29 (s, 0.090H), 8.12 (s, 0.10H), 7.83-7.60 (m, 1.90H), 7.30-7.24 (m, 4.20H), 5.59 (d, 0.92H), 4.40-4.20 (m, 2.25H), 4.00-3.55 (m, 6.38H), 3.50-3.30 (m, 1.04H), 3.26 (s, 0.72H), 3.22 (s, 2.97H), 2.55-2.30 (m, 2.37H), 2.15-2.05 (m, 1.11H), 2.00-1.80 (m, 2.36H), 1.60-1.40 (m, 1.14H), 1.45-1.10 (m, 6.53H), 0.90-0.80 (m, 2.95H), 1.80-1.65 (m, 0.72H).

13C-NMR (CDCl₃, rotamers): δ 200.68, 174.39, 161.62, 155.84, 155.56, 140.60, 134.69, 123.79, 123.38, 122.94, 122.43, 119.01, 109.47, 70.83, 70.70, 70.14, 59.27, 59.06, 59.01, 54.68, 52.70, 50.46, 48.60, 46.89, 44.41, 43.85, 33.42, 31.82, 28.89, 28.70, 25.17, 22.94, 22.63, 21.82, 13.90, 13.78.

ESI-MS: calcd. for C₂₂H₃₂N₄O₄ (416.52); found (pos.): 417.2 [M+H]. found (neg.): 415.5 [M−H].

### Example 33

### N-Hydroxy-N—((R)-2-{(S)-2-[1-(2-hydroxy-ethyl)-1H-benzoimidazol-2-yl]-pyrrolidine-1-carbonyl}-hexyl)-formamide

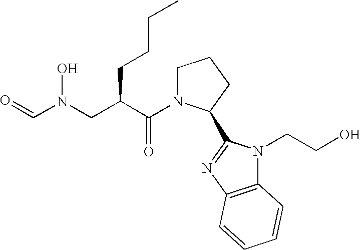

The title compound is prepared from (R)-2-[(Benzyloxy-formyl-amino)-methyl]-hexanoic acid A-7 (preparation is described in General Procedure A) and 2-((S)-2-Pyrrolidin-2-yl-benzoimidazol-1-yl)-ethanol N-3 (preparation is described in General Procedure N, PG=CBz) according to General Procedure B.

1H-NMR (CDCl₃, rotamers): δ 10.48 (s, 0.50H), 8.22 (s, 0.97H), 7.70-7.60 (m, 1.03H), 7.30-7.05 (m, 3H), 6.86 (s. 0.55H), 5.68 (d, 0.68H), 5.55-5.30 (m, 0.27H), 5.90-5.75 (br, 0.13H), 4.40-4.15 (m, 1.88H), 4.15-3.50 (m, 5.47H), 3.42-3.25 (m, 0.80H), 3.18-3.05 (m, 0.18H), 3.04-2.87 (m, 0.11H), 2.77-2.62 (m, 0.62H), 2.42-2.20 (m, 1.42H), 2.10-1.75 (m, 2.63H), 1.64-1.05 (m, 5.91H), 0.90-0.74 (m, 2.84H).

13C-NMR (CDCl₃, rotamers): δ 173.94, 172.56, 162.07, 161.72, 156.62, 156.25, 155.92, 142.75, 140.76, 134.67, 134.31, 123.79, 123.35, 122.76, 122.69, 122.29, 122.14, 119.46, 119.20, 109.84, 109.43, 109.26, 61.94, 61.84, 59.64, 55.13, 53.71, 51.10, 49.79, 47.94, 47.30, 47.24, 46.64, 46.33, 43.21, 41.62, 33.15, 31.98, 31.38, 29.86, 29.16, 28.99, 28.67, 25.02, 22.81, 22.73, 22.62, 21.54, 13.89.

ESI-MS: calcd. for C₂₁H₃₀N₄O₄ (402.50); found (pos.): 403.3 [M+H]. found (neg.): 401.5[M−H].

### Example 34

### N-{(R)-2-Cyclopentylmethyl-3-[(S)-2-(1H-naphtho[2,3-d]imidazol-2-yl)-pyrrolidin-1-yl]-3-oxo-propyl}-N-hydroxy-formamide

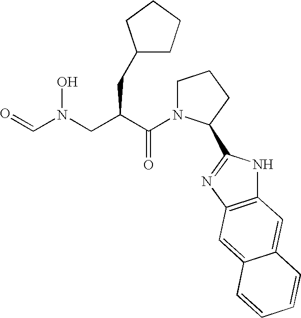

The title compound is prepared from (R)-2-[(Benzyloxy-formyl-amino)-methyl]-3-cyclopentyl-propionic acid A-7 (preparation is described in General Procedure A) and (S)-2-Pyrrolidin-2-yl-1H-naphtho[2,3-d]imidazole E-5 (preparation is described in General Procedure E; PG=CBz) according to General Procedure B.

1H-NMR (DMSO-d₄, rotamers): δ 9.07 (s, 0.25H), 8.92-8.66 (m, 4.09H), 8.70 (s, 0.51H), 8.60 (s, 0.14H), 8.20-8.00 (m, 1.97H), 6.22-6.14 (m, 0.28H), 5.98 (br.s, 0.72H), 4.91-4.76 (m, 1.58H), 4.64-4.47 (m, 1.69H), 4.47-4.28 (m, 1.75H), 3.13-2.94 (m, 1.58H), 2.94-2.65 (m, 2.10H), 2.65-2.46 (m, 2.63H), 2.46-2.10 (m, 6.68H), 2.09-2.00 (m, 0.73H), 1.95-1.62 (m, 2.56H), 1.46-1.32 (m, 1.03H), 1.26-1.00 (m, 2.06H).

13C-NMR (DMSO-d₄, rotamers): δ172.0, 157.3, 143.8, 135.0, 129.8, 128.0, 127.3, 123.5, 122.8, 114.6, 106.4, 54.9, 51.7, 48.6, 46.9, 46.3, 36.9, 32.6, 32.3, 30.9, 24.8, 24.4.

ES-MS: calcd. for C₂₅H₃₀N₄O₃ (434.23); found (pos.): 436.0 [M+H]; found (neg.): 433.4 [M−H].

### Example 35

### N-Hydroxy-N-{(R)-2-[(S)-2-(1H-naphtho[2,3-d]imidazol-2-yl)-pyrrolidine-1-carbonyl]-pentyl}-formamide

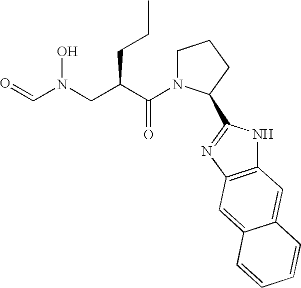

The title compound is prepared from (R)-2-[(Benzyloxy-formyl-amino)-methyl]-pentanoic acid A-7 (preparation is described in General Procedure A) and (S)-2-Pyrrolidin-2-yl-1H-naphtho[2,3-d]imidazole E-5 (preparation is described in General Procedure E; PG=CBz) according to General Procedure B.

1H-NMR (MeOH-d₄, rotamers): δ 8.29 (s, 0.21H), 8.15 (s, 0.17H), 8.08-7.83 (m, 4.28H), 7.81-7.72 (m, 0.19H), 7.42-7.27 (m, 1.86H), 5.51-5.43 (m, 0.35H), 5.23-5.21 (m, 0.65H), 4.00-3.82 (m, 1.46H), 3.82-3.60 (m, 2.02H), 3.51-3.38 (m, 0.83H), 3.18-3.09 (m, 0.23H), 2.99-2.84 (m, 0.29H), 2.76-2.65 (m, 0.19H), 2.58-2.00 (m, 3.67H), 1.98-1.76 (m, 0.52H), 1.67-1.23 (m, 4.12H), 0.99-0.84 (m, 3H).

13C-NMR (MeOH-d₄, rotamers): δ 131.9, 129.7, 128.2, 125.7, 125.6, 124.1, 49.9, 47.0.

ES-MS: calcd. for C₂₂H₂₆N₄O₃ (394.20); found (pos.): 395.9 [M+H]; found (neg.): 393.6 [M−H].

### Example 36

### N-Hydroxy-N-{(R)-2-[(S)-2-(1H-naphtho[2,3-d]imidazol-2-yl)-pyrrolidine-1-carbonyl]-hexyl}-formamide

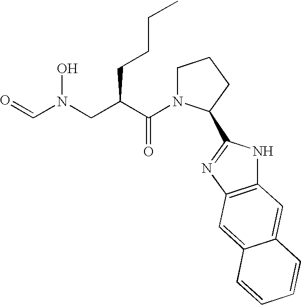

The title compound is prepared from (R)-2-[(Benzyloxy-formyl-amino)-methyl]-hexanoic acid A-7 (preparation is described in General Procedure A) and (S)-2-Pyrrolidin-2-yl-1H-naphtho[2,3-d]imidazole E-5 (preparation is described in General Procedure E; PG=CBz) according to General Procedure B.

1H-NMR (MeOH-d₄, rotamers): δ 8.20 (s, 0.19H), 8.03 (s, 0.32H), 7.90 (s, 1.62H), 7.89-7.82 (m, 1.92H), 7.75 (s, 0.49H), 7.68 (s, 0.15H), 7.34-7.25 (m, 1.93H), 5.39 (d, J=7.8, 0.29H), 5.25-5.05 (m, 0.71H), 3.89-3.75 (m, 1.52H), 3.72-3.52 (m, 1.73H), 3.45-3.29 (m, 0.73H), 3.10-2.97 (m, 0.19H), 2.89-2.75 (m, 0.34H), 2.67-2.53 (m, 0.15H), 2.51-2.30 (m, 1.28H), 2.29-2.00 (m, 2.39H), 1.99-1.87 (m, 0.32H) 1.87-1.77 (m, 0.30H), 1.76-1.68 (m, 0.24H), 1.6-1.47 (m, 1.05H), 1.45-1.31 (m, 1.28H), 1.29-1.11 (m, 5H), 0.87-0.81 (m, 1.15H), 0.77-0.70 (m, 1.98H).

ES-MS: calcd. for C₂₃H₂₈N₄O₃ (408.50); found (pos.): 409.9 [M+H].

### Example 37

### N-Hydroxy-N-{(R)-2-[(S)-2-(1H-naphtho[2,3-d]imidazol-2-yl)-pyrrolidine-1-carbonyl]-heptyl}-formamide

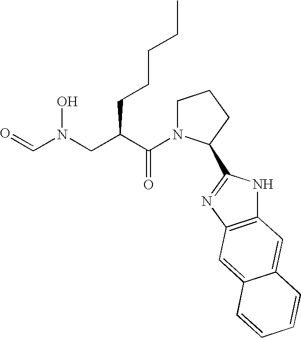

The title compound is prepared from (R)-2-[(Benzyloxy-formyl-amino)-methyl]-heptanoic acid A-7 (preparation is described in General Procedure A) and (S)-2-Pyrrolidin-2-yl-1H-naphtho[2,3-d]imidazole E-5 (preparation is described in General Procedure E; PG=CBz) according to General Procedure B.

1H-NMR (MeOH-d₄, rotamers): δ 8.00-7.65 (m, 4H), 7.35-7.17 (m, 2H), 5.40-5.30 (m, 0.32H), 5.24-5.08 (m, 0.68H), 3.88-3.75 (m, 1.82H), 3.70-3.50 (m, 2.13H), 3.45-3.27 (m, 1.05H), 2.45-2.28 (m, 1.39H), 2.28-1.90 (m, 2.61H), 1.56-0.98 (m, 8H), 0.90-0.60 (m, 3H).

13C-NMR (MeOH-d₄, rotamers): δ 173.9, 162.6, 162.0, 161.5, 160.2, 158.3, 137.6, 130.6, 127.5, 123.7, 116.2, 110.5, 102.2, 56.4, 55.5, 51.9, 47.8, 47.0, 46.5, 41.6, 31.7, 31.4, 29.7, 26.2, 24.6, 22.3, 22.1, 13.0, 12.9.

ES-MS: calcd. for C₂₄H₃₀N₄O₃ (422.53); found (pos.): 423.6 [M+H]; found (neg.): 421.6 [M−H], 535.6 [M+CF₃CO₂].

### Example 38

### N-{(R)-2-Benzyl-3-[(S)-2-(1H-naphtho[2,3-d]imidazol-2-yl)-pyrrolidine-1-yl]-3-oxo-propyl}-N-hydroxy-formamide

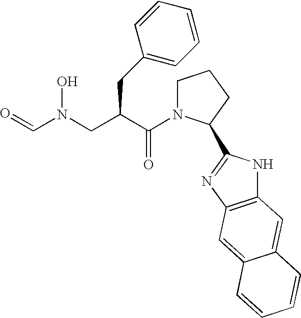

The title compound is prepared from (R)-2-[(Benzyloxy-formyl-amino)-methyl]-3-phenyl-propionic acid A-7 (preparation is described in General Procedure A) and (S)-2-Pyrrolidin-2-yl-1H-naphtho[2,3-d]imidazole E-5 (preparation is described in General Procedure E; PG=CBz) according to General Procedure B.

1H-NMR (MeOH-d₄, rotamer): δ 8.18 (m, 1H), 7.97-7.68 (m, 3.38H), 7.35-7.00 (m, 6.01H), 7.00-6.78 (m, 1.60H), 5.25-5.18 (m, 1H), 4.35-4.20 (m, 0.53H), 3.98-3.88 (d, 0.33H), 3.85-3.60 (m, 1.30H), 3.60-3.22 (m, 2.83H), 2.90-2.50 (m, 4H), 1.30-1.00 (m, 2H).

ES-MS: calcd. for C₂₆H₂₆N₄O₃ (442.52); found (pos.): 444.1 [M+1]; found (neg.): 442.3 [M-1], 556.1 [M+CF₃CO₂].

### Example 39

### N—[(R)-2-((S)-2-Benzooxazol-2-yl-pyrrolidine-1-carbonyl)-hexyl]-N-hydroxy-formamide

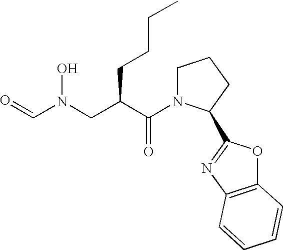

The title compound is prepared from (R)-2-[(Benzyloxy-formyl-amino)-methyl]-hexanoic acid A-7 (preparation is described in General Procedure A) and (S)-2-Pyrrolidin-2-yl-benzooxazole F-5 (preparation is described in General Procedure F) according to General Procedure B.

1H-NMR (MeOH-d₄, rotamers): δ 8.25 (s, 0.24H), 0.78 (s, 0.76H), 7.65-7.55 (m, 1.02H), 7.55-7.47 (m, 0.86H), 7.40-7.25 (m, 2.13H), 5.30-5.15 (m, 1H), 3.95-3.78 (m, 2.15H), 3.78-3.60 (m, 1.89H), 3.50-3.37 (m, 0.96H), 2.55-2.30 (m, 1.11H), 2.30-1.95 (m, 2.90H), 1.70-1.18 (m, 6H), 1.00-0.80 (m, 3H).

13C-NMR (MeOH-d₄, rotamers): δ 175.3, 175.1, 168.2, 163.0, 160.7, 158.7, 151.8, 141.9, 127.2, 127.1, 126.6, 126.5, 125.6, 125.0, 124.9, 121.3, 119.7, 112.5, 112.4, 110.8, 57.2, 55.8, 53.4, 47.3, 43.5, 42.3, 31.8, 31.2, 30.5, 30.1, 25.7, 23.9, 15.0, 13.8.

ES-MS: calcd. for C₁₉H₂₅N₃O₄ (359.43); found (pos.): 360.8 [M+H].

### Example 40

### N—[(R)-2-((S)-2-Benzooxazol-2-yl-pyrrolidine-1-carbonyl)-heptyl]-N-hydroxy-formamide

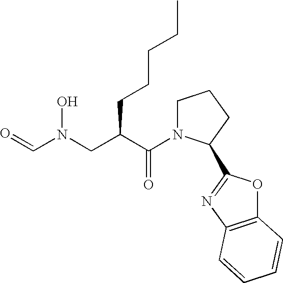

The title compound is prepared from (R)-2-[(Benzyloxy-formyl-amino)-methyl]-heptanoic acid A-7 (preparation is described in General Procedure A) and (S)-2-Pyrrolidin-2-yl-benzooxazole F-5 (preparation is described in General Procedure F) according to General Procedure B.

1H-NMR (MeOH-d₄, rotamers): δ 8.28 (s, 0.32H), 7.83 (s, 0.68H), 7.67-7.55 (m, 1.02H), 7.55-7.42 (m, 0.87H), 7.42-7.25 (m, 2.11H), 5.50-5.40 (m, 0.19H), 5.28-5.17 (m, 0.81H), 3.97-3.82 (m, 2.16H), 3.82-3.60 (m, 1.87H), 3.50-3.35 (m, 0.97H), 2.54-2.34 (m, 1.14H), 2.34-2.00 (m, 2.86H), 1.70-1.15 (m, 8H), 0.97-0.80 (m, 3H).

13C-NMR (MeOH-d₄, rotamers): δ 175.1, 168.2, 160.7, 158.7, 142.1, 127.2, 126.6, 126.5, 125.5, 125.0, 124.9, 121.3, 119.7, 112.5, 110.8, 107.5, 57.2, 55.8, 47.31, 43.6, 42.3, 33.1, 31.8, 27.5, 25.7, 23.6, 15.0, 13.7.

ES-MS: calcd. for C₂₀H₂₇N₃O₄ (373.46); found (pos.): 375.0 [M+H]; found (neg.): 373.0 [M−H].

### Example 41

### N—[(R)-3-((S)-2-Benzooxazol-2-yl-pyrrolidine-1-yl)-2-benzyl-3-oxo-propyl]-N-hydroxy-formamide

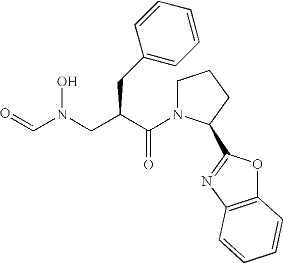

The title compound is prepared from (R)-2-[(Benzyloxy-formyl-amino)-methyl]-3-phenyl-propionic acid A-7 (preparation is described in General Procedure A) and (S)-2-Pyrrolidin-2-yl-benzooxazole F-5 (preparation is described in General Procedure F) according to General Procedure B.

1H-NMR (MeOH-d₄, rotamers): δ 8.18 (s, 0.22H), 7.78-7.68 (d, 0.78H), 7.67-7.37 (m, 1.98H), 7.35-7.15 (m, 3.92H), 7.15-6.95 (m, 3.09H), 5.39 (s, 0.38H), 5.18-5.08 (m, 0.62H), 4.28-4.17 (d, 0.42H), 3.80-3.55 (m, 1.71H), 3.55-3.35 (m, 1.75H), 3.35-3.26 (m, 1.12H), 2.87-2.70 (m, 0.94H), 2.70-2.55 (m, 0.96H), 2.10-1.95 (m, 2.10H), 1.80-1.60 (m, 1.26H), 1.55-1.35 (m, 0.74H).

13C-NMR (MeOH-d₄, rotamers): δ 174.1, 167.8, 163.8, 159.8, 151.9, 141.9, 139.7, 130.2, 130.1, 129.9, 129.8, 129.6, 129.5, 128.2, 127.7, 127.6, 126.9, 126.4, 126.1, 126.0, 125.8, 120.9, 120.6, 112.1, 111.9, 56.6, 56.3, 53.4, 47.3, 46.3, 45.2, 38.3, 37.4, 37.0, 32.2, 31.5, 25.5, 23.2.

ES-MS: calcd. for C₂₂H₂₃N₃O₄ (393.45); found (pos.): 395.1 [M+H].

### Example 42

### N—[(R)-3-((S)-2-Benzooxazol-2-yl-pyrrolidine-1-yl)-2-cyclopentylmethyl-3-oxo-propyl]-N-hydroxy-formamide

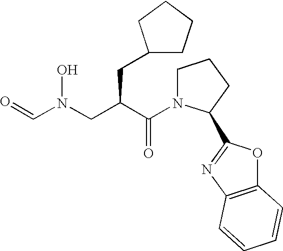

The title compound is prepared from (R)-2-[(Benzyloxy-formyl-amino)-methyl]-3-cyclopentyl-propionic acid A-7 (preparation is described in General Procedure A) and (S)-2-Pyrrolidin-2-yl-benzooxazole F-5 (preparation is described in General Procedure F) according to General Procedure B.

1H-NMR (MeOH-d₄, rotamers): δ 8.19 (s, 0.24H), 7.74 (s, 0.58H), 7.69 (s, 0.07H), 7.65-7.58 (m, 0.17H), 7.55-7.47 (m, 0.94H), 7.45-7.38 (m, 0.84H), 7.35-7.22 (m, 1.97H), 5.40-5.30 (m, 0.15H), 5.18-5.11 (m, 0.85H), 3.87-3.71 (m, 1.76H), 3.71-3.47 (m, 1.56H), 3.42-3.32 (m, 0.78H), 3.27-3.21 (m, 1.17H), 3.12-2.99 (m, 0.36H), 2.44-2.26 (m, 1.13H), 2.18-1.90 (m, 2.96H), 1.86-1.49 (m, 2.09H), 1.49-1.24 (m, 6.16H), 1.14-0.89 (m, 2.12H).

13C-NMR (MeOH-d₄, rotamers): δ 175.2, 168.1, 159.6, 151.8, 142.0, 126.4, 125.8, 120.5, 111.6, 99.5, 56.5, 53.8, 42.5, 38.8, 37.4, 34.2, 33.7, 31.8, 26.2, 25.7.

ES-MS: calcd. for C₂₁H₂₇N₄O₃ (385.20); found (pos.): 386.5 [M+H]; found (neg.): 384.4 [M−H], 769.7 [2M].

### Example 43

### N—[(R)-2-((S)-2-Benzooxazol-2-yl-pyrrolidine-1-carbonyl)-pentyl]-N-hydroxy-formamide

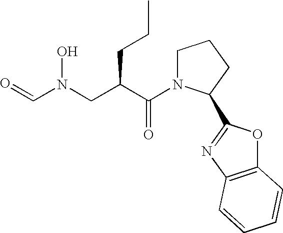

The title compound is prepared from (R)-2-[(Benzyloxy-formyl-amino)-methyl]-pentanoic acid A-7 (preparation is described in General Procedure A) and (S)-2-Pyrrolidin-2-yl-benzooxazole F-5 (preparation is described in General Procedure F) according to General Procedure B.

1H-NMR (MeOH-d₄, rotamers): δ 8.19 (s, 0.24H), 7.76-7.67 (m, 0.68H), 7.67-7.58 (m, 0.16H), 7.58-7.50 (m, 0.89H), 7.49-7.43 (m, 0.76H), 7.37-7.24 (m, 1.84H), 5.40-5.35 (m, 0.19H), 5.16-5.10 (m, 0.81H), 3.86-3.71 (m, 1.66H), 3.71-3.48 (m, 1.53H), 3.42-3.31 (m, 0.84H), 3.25 (s, 1.47H), 3.12-2.94 (m, 0.40H), 2.42-2.24 (m, 1.13H), 2.17-1.91 (m, 2.70H), 1.56-1.18 (m, 3.87H), 0.90-0.80 (m, 2.79H).

13C-NMR (MeOH-d₄, rotamers): δ 159.7, 151.8, 141.9, 126.3, 125.8, 120.5, 111.6, 56.5, 53.3, 42.8, 33.5, 31.8, 25.7, 21.1, 14.5.

ES-MS: calcd. for C₁₈H₂₃N₃O₄ (345.17); found (pos.): 346.8 [M+H]; found (neg.): 344.6 [M−H].

### Example 44

### N-{(R)-2-[(S)-2-(5-Ethanesulfonyl-benzooxazol-2-yl)-pyrrolidine-1-carbonyl]-hexyl}-N-hydroxy-formamide

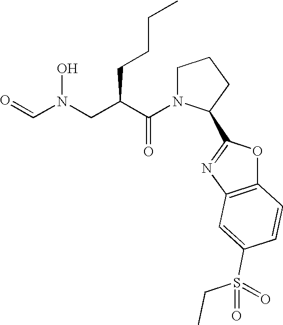

The title compound is prepared from (R)-2-[(Benzyloxy-formyl-amino)-methyl]-hexanoic acid A-7 (preparation is described in General Procedure A) and 5-Ethanesulfonyl-2-(S)-pyrrolidin-2-yl-benzooxazole F-5 (preparation is described in General Procedure F) according to General Procedure B.

1H-NMR (MeOH-d₄, rotamers): δ 8.28 (s, 0.38H), 8.10 (s, 0.62H), 7.98-7.80 (m, 1.20H), 7.80-7.70 (m, 1.80H), 5.30-5.10 (m, 1H), 3.94-3.80 (m, 2.19H), 3.80-3.63 (m, 2.27H), 3.50-3.37 (m, 1.27H), 3.15-3.00 (m, 1.27H), 2.55-2.30 (m, 1.13H), 2.28-2.02 (m, 2.87H), 1.67-1.23 (m, 6H), 1.23-1.14 (m, 3H), 0.97-0.82 (m, 3H).

ES-MS: calcd. for C₂₁H₂₉N₃O₆S (451.55); found (pos.): 452.8 [M+H]; found (neg.): 450.7 [M−H].

### Example 45

### N-{(R)-2-[(S)-2-(5-tert-Butyl-benzooxazol-2-yl)-pyrrolidine-1-carbonyl]-hexyl}-N-hydroxy-formamide

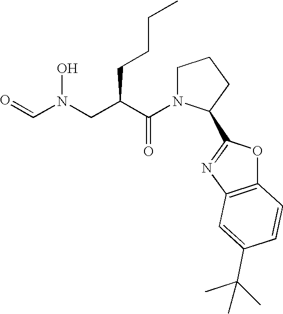

The title compound is prepared from (R)-2-[(Benzyloxy-formyl-amino)-methyl]-hexanoic acid A-7 (preparation is described in General Procedure A) and 5-tert-Butyl-2-(S)-pyrrolidin-2-yl-benzooxazole F-5 (preparation is described in General Procedure F) according to General Procedure B.

1H-NMR (MeOH-d₄, rotamers): δ 8.28 (s, 0.27H), 7.83 (s, 0.79H), 7.62 (1H), 7.55-7.50 (m, 0.32H), 7.44 (s, 2.21H), 5.30-5.15 (m, 1.05H), 3.95-3.85 (m, 2.28H), 3.70-3.65 (m, 1.83H), 3.50-3.40 (m, 0.98H), 2.50-2.34 (m, 1.40H), 2.30-2.05 (m, 3.87H), 1.65-1.43 (m, 2.87H), 1.43-1.25 (m, 17.89H), 0.94-0.83 (m, 4.00H).

13C-NMR (MeOH-d₄, rotamers): δ175.01, 168.33, 164.01, 159.70, 149.84, 149.54, 141.85, 124.73, 124.06, 117.44, 116.85, 110.81, 110.81, 56.54, 53.37, 42.90, 35.77, 32.14, 31.81, 31.12, 30.07, 25.69, 23.88, 14.39.

ESI-MS: calcd. for C₂₃H₃₃N₃O₄ (415.25); found (pos.): 416.4 [M+H]; found (neg.): 414.6[M−H].

### Example 46

### N-{(R)-2-[(S)-2-(6-Chloro-benzooxazol-2-yl)-pyrrolidine-1-carbonyl]-hexyl}-N-hydroxy-formamide

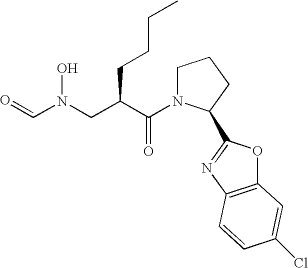

The title compound is prepared from (R)-2-[(Benzyloxy-formyl-amino)-methyl]-hexanoic acid A-7 (preparation is described in General Procedure A) and 6-Chloro-2-(S)-pyrrolidin-2-yl-benzooxazole F-5 (preparation is described in General Procedure F) according to General Procedure C.

1H-NMR (CDCl₃, rotamers): δ 9.94 (s, 0.13H), 9.40-8.98 (br, 0.49H), 8.26 (0.17H), 8.12 (s, 0.12H), 7.86-7.71 (m, 0.71H), 7.54-7.42 (m, 0.95H), 7.42-7.32 (m, 0.50H), 7.32-7.15 (m, 1.10H), 5.30-5.18 (m, 0.72H), 5.18-5.15 (d, J=7.68, 0.25H), 3.94-3.53 (m, 3.31H), 3.39-3.23 (m, 0.64H), 3.21-3.10 (m, 0.52H), 3.00-2.82 (m, 0.29H), 2.55-1.94 (m, 3.88H), 1.94-1.05 (m, 7.67H), 0.90-0.65 (m, 3.14H).

13C-NMR (CDCl₃, rotamers): δ 175.68, 172.37, 167.17, 166.30, 161.75, 161.44, 156.89, 156.62, 150.74, 139.96, 130.71, 130.39, 126.30, 125.83, 125.17, 124.93, 121.02, 120.56, 120.44, 119.91, 111.89, 111.49, 111.26, 111.20, 55.57, 55.16, 54.80, 51.27, 47.69, 47.12, 46.45, 46.42, 44.04, 43.28, 41.01, 31.37, 30.68, 30.54, 30.01, 29.91, 29.26, 28.89, 28.80, 24.74, 22.80, 22.76, 22.72, 13.96, 13.85.

ESI-MS: calcd. for C19H24ClN30 (393.14); found (pos.): 394.8 [M+H].

### Example 47

### N-{(R)-2-[(S)-2-(5,6-Difluoro-benzooxazol-2-yl)-pyrrolidine-1-carbonyl]-hexyl}-N-hydroxy-formamide

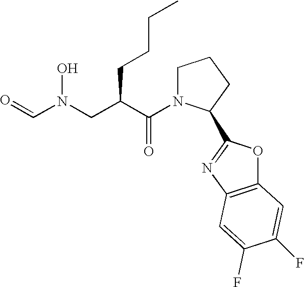

The title compound is prepared from (R)-2-[(Benzyloxy-formyl-amino)-methyl]-hexanoic acid A-7 (preparation is described in General Procedure A) and 5,6-Difluoro-2-(S)-pyrrolidin-2-yl-benzooxazole F-5 (preparation is described in General Procedure F) according to General Procedure B.

1H-NMR (CDCl₃, rotamers): δ 10.15-9.53 (br, 0.46H), 9.15 (br, 0.10H), 8.33 (s, 0.12H), 8.18 (s, 0.06H), 7.75 (s, 0.97H), 7.64-7.53 (m, 0.08H), 7.53-7.38 (m, 0.90H), 7.38-7.25 (m, 0.76H), 5.38-5.30 (m, 0.80H), 5.30-5.22 (m, 0.15H), 3.97-3.62 (m, 3.21H), 3.48-3.32 (m, 0.77H), 3.32-3.19 (m, 0.23H), 2.62-2.30 (m, 1.28H), 2.29-1.98 (m, 3.01H), 1.93-1.80 (br, 0.1H), 1.70-1.52 (m, 1.20H), 1.54-1.27 (m, 4.95H), 0.97-0.83 (t, J=6.8 Hz).

13C-NMR (CDCl₃, rotamers): δ 172.42, 168.32, 168.29, 161.55, 157.38, 156.94, 149.92, 149.77, 147.47, 147.42, 147.31, 147.27, 145.58, 145.57, 145.46, 145.45, 136.67, 136.56, 107.56, 107.46, 107.35, 99.88, 99.79, 99.65, 99.56, 55.27, 55.09, 54.77, 51.61, 47.63, 47.35, 47.06, 46.42, 43.10, 40.90, 30.71, 30.63, 30.54, 30.00, 29.96, 29.14, 29.85, 29.14, 28.85, 28.74, 24.68, 22.78, 22.74, 22.70, 22.67, 13.92, 13.80.

ESI-MS: calcd. for C19H23F2N3O4 (395.17); found (pos.): 396.5 [M+H]. found (neg.): 394.8[M−H].

### Example 48

### N-Hydroxy-N-{(R)-2-[(S)-2-(5-phenyl-benzooxazol-2-yl)-pyrrolidine-1-carbonyl]-hexyl}-formamide

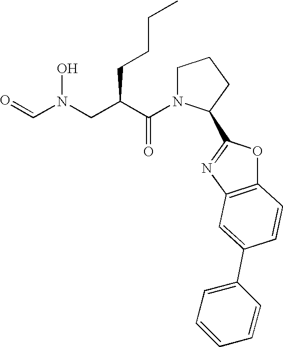

The title compound is prepared from (R)-2-[(Benzyloxy-formyl-amino)-methyl]-hexanoic acid A-7 (preparation is described in General Procedure A) and 5-Phenyl-2-(S)-pyrrolidin-2-yl-benzooxazole F-5 (preparation is described in General Procedure F) according to General Procedure B.

1H-NMR (CDCl₃, rotamers): δ 8.33 (s, 0.18H), 8.21 (s, 0.26H), 8.04 (s, 0.14H), 7.85-7.78 (m, 1.22H), 7.65-7.30 (m, 6.53H), 5.32-5.22 (m, 0.53H), 5.26 (d, J=7.5, 0.47H), 4.00-3.65 (m, 3.38H), 3.48-3.34 (m, 0.52H), 3.27-3.18 (m, 0.35H), 3.06-2.92 (m, 0.37H), 2.64-2.01 (m, 4.26H), 2.00-1.20 (m, 8.38H), 0.94-0.82 (m, 2.89). 13C-NMR (CDCl₃, rotamers): δ 172.3, 167.1, 161.8, 161.3, 156.1, 150.1, 141.6, 139.8, 138.3, 129.0, 128.9, 128.8, 127.7, 127.5, 127.2, 125.9, 124.7, 124.5, 118.6, 118.5, 117.7, 111.2, 110.6, 55.7, 54.9, 50.8, 48.0, 47.2, 46.4, 44.1, 41.2, 32.2, 31.5, 30.8, 30.6, 30.1, 29.9, 29.3, 28.9, 28.9, 24.8, 22.8, 22.3.

ES-MS: calcd. for C₂₅H₂₉N₃O₄ (435.22); found (pos.): 436.7 [M+H]; found (neg.): 434.5.

### Example 49

### N-Hydroxy-N—[(R)-2-((S)-2-oxazolo[4,5-b]pyridin-2-yl-pyrrolidine-1-carbonyl)-hexyl]-formamide

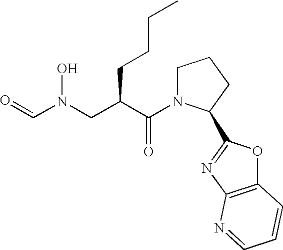

The title compound is prepared from (R)-2-[(Benzyloxy-formyl-amino)-methyl]-hexanoic acid A-7 (preparation is described in General Procedure A) and (S)-2-Pyrrolidin-2-yl-oxazolo[4,5-b]pyridine F-5 (preparation is described in General Procedure F) according to General Procedure B.

1H-NMR (CDCl₃, rotamers): δ 8.61-8.48 (m, 0.83H), 8.35 (d, J=13.0, 0.24H), 7.90-7.71 (m, 1.01H), 7.40-7.27 (m, 0.43H), 4.97-4.71 (m, 1H), 4.12-3.55 (m, 3.15H), 3.51-3.40 (m, 0.61H), 3.25-3.10 (m, 0.23H), 2.62-1.98 (m, 3.86H), 1.20-1.94 (m, 14.19H), 0.94-0.82 (m, 2.40).

13C-NMR (CDCl₃, rotamers): δ 173.8, 172.5, 169.9, 169.8, 163.5, 161.4, 156.2, 147.1, 146.6, 146.4, 120.6, 120.2, 119.9, 118.8, 118.4, 118.3, 55.4, 55.0, 54.8, 52.3, 50.7, 47.5, 47.1, 42.6, 41.1, 30.7, 30.6, 30.3, 29.9, 29.7, 29.6, 29.0, 28.9, 24.7, 24.4, 22.8, 22.7.

ES-MS: calcd. for C₁₈H₂₄N₄O₄ (360.18); found (pos.): 361.2 [M+H]; found (neg.): 359.2.

### Example 50

### N-Hydroxy-N—[(R)-2-((S)-2-naphtho[2,3-d]oxazol-2-yl-pyrrolidine-1-carbonyl)-hexyl]-formamide

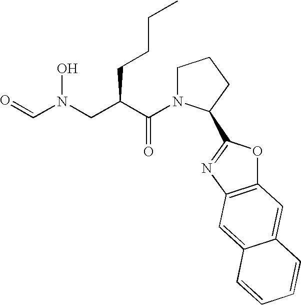

The title compound is prepared from (R)-2-[(Benzyloxy-formyl-amino)-methyl]-hexanoic acid A-7 (preparation is described in General Procedure A) and (S)-2-Pyrrolidin-2-yl-naphtho[2,3-d]oxazole F-5 (preparation is described in General Procedure F) according to General Procedure B.

1H-NMR (MeOH-d₄, rotamers): δ 8.54-8.46 (m, 0.19H), 8.32-8.23 (m, 0.36H), 8.10-7.91 (m, 4.57H), 7.85 (s, 0.82H), 7.79 (s, 0.17H), 7.54-7.42 (m, 1.85H), 7.14 (s, 0.21H), 5.31-5.22 (m, 1.0H), 3.98-3.85 (m, 2.01H), 3.85-3.61 (m, 2.56H), 3.52-3.41 (m, 1.07H), 2.56-2.39 (m, 1.36H), 2.31-2.00 (m, 4.29H), 1.68-1.27 (m, 8.63H), 1.00-0.82 (m, 4.27H).

13C-NMR (MeOH-d₄, rotamers): δ175.1, 170.5, 164.0, 159.7, 150.7, 141.9, 133.1, 132.9, 129.5, 129.0, 126.7, 126.0, 117.8, 107.5, 56.7, 53.4, 42.9, 31.7, 31.1, 30.1, 25.8, 23.9, 14.4, 14.2.

ES-MS: calcd. for C₂₃H₂₇N₃O₄ (409.20); found (pos.): 411.0 [M+H]; found (neg.): 408.6 [M−H].

### Example 51

### N-Hydroxy-N—[(R)-2-((S)-2-naphtho[2,3-d]oxazol-2-yl-pyrrolidine-1-carbonyl)-heptyl]-formamide

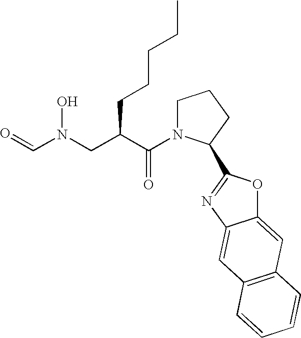

The title compound is prepared from (R)-2-[(Benzyloxy-formyl-amino)-methyl]-hexanoic acid A-7 (preparation is described in General Procedure A) and (S)-2-Pyrrolidin-2-yl-naphtho[2,3-d]oxazole F-5 (preparation is described in General Procedure F) according to General Procedure B.

1H-NMR (MeOH-d₄, rotamers): δ 8.40 (s, 0.34H), 8.28 (s, 0.66H), 8.08-7.92 (m, 3.24H), 7.92-7.87 (m, 0.61H), 7.53-7.40 (m, 1.60H), 7.28-7.18 (m, 0.56H), 5.48 (s, 0.45H), 5.30-5.20 (m, 0.55H), 3.97-3.83 (m, 1.80H), 3.82-3.65 (m, 2.20H), 3.52-3.40 (m, 1.02H), 2.55-2.35 (m, 1H), 2.35-2.07 (m, 3H), 1.68-1.17 (m, 8H), 0.97-0.75 (m, 3H).

13C-NMR (MeOH-d₄, rotamers): δ 175.1, 170.5, 159.7, 150.7, 141.9, 133.1, 132.9, 129.5, 129.0, 126.7, 126.0, 117.9, 107.5, 56.7, 53.4, 42.9, 33.0, 31.7, 31.4, 27.5, 25.8, 23.6, 23.4, 14.4.

ES-MS: calcd. for C₂₄H₂₉N₃O₄ (423.52); found (pos.):425.0 [M+H]; found (neg.): 422.6 [M−H], 536.6 [M+CF₃CO₂].

### Example 52

### N—[(R)-2-Benzyl-3-((S)-2-naphtho[2,3-d]oxazol-2-yl-pyrrolidine-1-yl]-3-oxo-propyl]-N-hydroxy-formamide

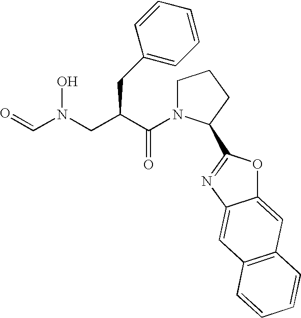

The title compound is prepared from (R)-2-[(Benzyloxy-formyl-amino)-methyl]-3-phenyl-propionic acid A-7 (preparation is described in General Procedure A) and (S)-2-Pyrrolidin-2-yl-naphtho[2,3-d]oxazole F-5 (preparation is described in General Procedure F) according to General Procedure B.

1H-NMR (MeOH-d₄, rotamers): δ 8.55 (s, 0.34H), 8.20-8.05 (m, 0.66H), 8.05-7.90 (m, 1.52H), 7.90-7.78 (m, 1.60H), 7.70-7.62 (m, 0.71H), 7.62-7.55 (m, 0.73H), 7.55-7.40 (m, 1.03H), 7.40-7.10 (m, 5.04H), 7.06-6.95 (m, 0.37H), 5.48 (s, 0.71H), 5.27-5.15 (m, 0.29H), 3.32-3.17 (m, 5H), 2.20-1.75 (m, 6H).

13C-NMR (MeOH-d₄, rotamers): δ 150.2, 139.5, 133.4, 132.9, 130.9, 130.2, 129.9, 129.6, 129.1, 128.8, 128.4, 127.8, 126.7, 126.0, 125.6, 124.9, 117.9, 111.8, 107.8, 105.6, 56.5, 54.8, 53.1, 47.4, 45.1, 37.9, 31.5, 25.6.

ES-MS: calcd. for C₂₆H₂₅N₃O₄ (443.51); found (pos.): 444.8 [M+H]; found (neg.): 442.6 [M−H], 556.6 [M+CF₃CO₂].

### Example 53

### N—[(R)-2-Cyclopentylmethyl-3-((S)-2-naphtho[2,3-d]oxazol-2-yl-pyrrolidine-1-yl)-3-oxo-propyl]-N-hydroxy-formamide

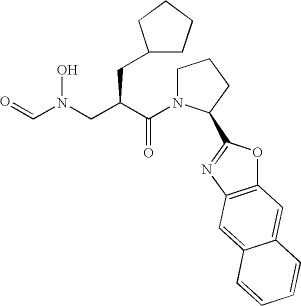

The title compound is prepared from (R)-2-[(Benzyloxy-formyl-amino)-methyl]-3-cyclopentyl-propionic acid A-7 (preparation is described in General Procedure A) and (S)-2-Pyrrolidin-2-yl-naphtho[2,3-d]oxazole F-5 (preparation is described in General Procedure F) according to General Procedure B.

1H-NMR (MeOH-d₄, rotamers): δ 8.54-8.49 (m, 0.15H), 8.31-8.25 (m, 0.24H), 8.21-8.13 (m, 0.12H), 8.10-8.03 (m, 0.83H), 8.01-7.91 (m, 2.27H), 7.84 (s, 0.59H), 7.79-7.75 (m, 0.07H), 7.74-7.70 (m, 0.06H), 7.67-7.63 (m, 0.17H), 7.60-7.54 (m, 0.18H), 7.54-7.43 (m, 1.60H), 7.34-7.20 (m, 0.38H), 7.17-7.11 (m, 0.17H), 5.47-5.44 (m, 0.30H), 5.32-5.24 (m, 0.70H), 4.00-3.87 (m, 1.42H), 3.82-3.62 (m, 1.65H), 3.52-3.41 (m, 0.77H), 3.41-3.31 (m, 1.16H), 2.55-2.37 (m, 0.94H), 2.34-2.04 (m, 3.06H), 1.98-1.48 (m, 7.91H), 1.46-1.32 (m, 1.03H), 1.26-1.00 (m, 2.06H).

13C-NMR (MeOH-d₄, rotamers): δ175.5, 175.2, 170.5, 159.7, 150.7, 144.7, 141.9, 133.1, 132.9, 129.5, 129.0, 126.7, 125.9, 117.9, 107.5, 56.7, 54.8, 53.8, 42.5, 38.8, 37.9, 37.9, 37.5, 34.2, 33.7, 31.7, 26.3, 26.2, 25.8.

ES-MS: calcd. for C₂₅H₂₉N₃O₄ (435.22); found (pos.): 437.0 [M+H]; found (neg.): 434.5 [M−H].

### Example 54

### N-Hydroxy-N—[(R)-2-((S)-2-naphtho[2,3-d]oxazol-2-yl-pyrrolidine-1-carbonyl)-pentyl]-formamide

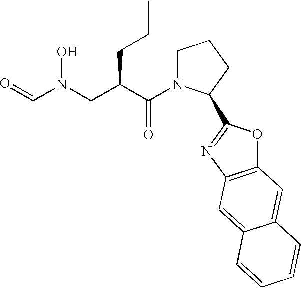

The title compound is prepared from (R)-2-[(Benzyloxy-formyl-amino)-methyl]-pentanoic acid A-7 (preparation is described in General Procedure A) and (S)-2-Pyrrolidin-2-yl-naphtho[2,3-d]oxazole F-5 (preparation is described in General Procedure F) according to General Procedure B.

1H-NMR (MeOH-d₄, rotamers): δ 8.54-8.46 (m, 0.19H), 8.30-8.25 (m, 0.31H), 8.22-8.16 (m, 0.18H), 8.12-7.91 (m, 4.49H), 7.84 (s, 0.80H), 7.69-7.62 (m, 0.22H), 7.62-7.53 (m, 0.22H), 7.53-7.44 (m, 2.33H), 7.32-7.20 (m, 0.51H), 7.14 (s, 0.21H), 5.30-5.22 (m, 1.0H), 4.00-3.83 (m, 1.99H), 3.83-3.62 (m, 2.52H), 3.54-3.49 (m, 1.08H), 2.56-2.38 (m, 1.38H), 2.31-2.00 (m, 4.32H), 1.69-1.32 (m, 5.62H), 1.01-0.91 (m, 4.14H).

13C-NMR (MeOH-d₄, rotamers): δ 175.1, 170.5, 159.7, 150.7, 141.9, 133.1, 132.9, 129.5, 129.0, 126.7, 126.0, 117.8, 107.5, 56.7, 54.8, 53.4, 42.8, 33.8, 33.5, 31.8, 25.8, 21.1, 14.5.

ES-MS: calcd. for C₂₂H₂₅N₃O₄ (395.18); found (pos.): 396.7 [M+H]; found (neg.): 394.7 [M−H].

### Example 55

### N—[(R)-2-((S)-2-Benzothiazol-2-yl-pyrrolidine-1-carbonyl)-hexyl]-N-hydroxy-formamide

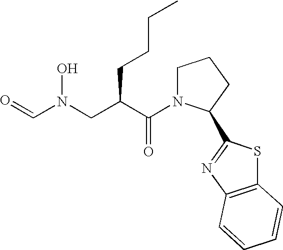

The title compound is prepared from (R)-2-[(Benzyloxy-formyl-amino)-methyl]-hexanoic acid A-7 (preparation is described in General Procedure A) and (S)-2-Pyrrolidin-2-yl-benzothiazole K-4 (preparation is described in General Procedure K) according to General Procedure C.

1H-NMR (MeOH-d₄, rotamers): δ 8.28 (s, 0.24H), 8.04-7.96 (m, 0.29), 7.96-7.80 (m, 2.30H), 7.76 (s, 0.11H), 7.44-7.24 (m, 0.96H), 5.76-5.64 (m, 0.03), 5.64-5.50 (m, 0.11H), 5.50-5.34 (m, 0.86H), 4.10-3.94 (m, 0.09H), 3.94-3.70 (m, 3.32H), 3.70-3.54 (m, 0.33H), 3.54-3.34 (m, 0.80H), 3.20-3.08 (m, 0.31H), 3.08-2.97 (m, 0.07), 2.97-2.80 (m, 0.06H), 2.60-2.46 (m, 0.16H), 2.46-2.30 (m, 1.06H), 2.30-1.90 (m, 3.42H), 1.76-1.57 (m, 0.98H), 1.57-1.14 (m, 5.97H), 1.0-0.82 (m, 3.27H).

ES-MS: calcd. for C₁₉H₂₅N₃O₃S (375.49); found (pos.): 376.4 [M+H].

### Example 56

### N—[(R)-3-((S)-2-Benzothiazol-2-yl-pyrrolidine-1-yl)-2-cyclopentylmethyl-3-oxo-propyl]-N-hydroxy-formamide

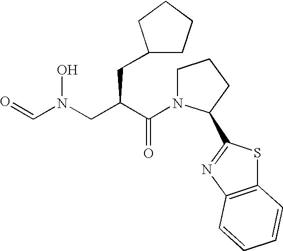

The title compound is prepared from (R)-2-[(Benzyloxy-formyl-amino)-methyl]-3-cyclopentyl-propionic acid A-7 (preparation is described in General Procedure A) and (S)-2-Pyrrolidin-2-yl-benzothiazole K-4 (preparation is described in General Procedure K) according to General Procedure C.

1H-NMR (MeOH-d₄, rotamers): δ 8.53 (s, 0.02H), 8.28 (s, 0.25H), 8.02-7.82 (m, 2.40H), 7.75 (s, 0.07H), 7.56-7.34 (m, 1.88H), 5.59-5.53 (m, 0.17H), 5.49-5.42 (m, 0.83H), 3.95-3.81 (m, 1.80H), 3.81-3.58 (m, 1.57H), 3.52-3.40 (m, 0.76H), 3.20-3.10 (m, 0.29H), 3.07-2.84 (m, 0.25H), 2.67 (s, 0.02H), 2.60-2.47 (m, 0.17H), 2.47-2.32 (m, 0.97H), 2.30-1.98 (3.05H), 1.98-1.45 (m, 8.26H), 1.45-1.01 (m, 3.53H), 0.92-0.80 (m, 0.02H).

13C-NMR (MeOH-d₄, rotamers): δ 175.7, 175.6, 164.0, 159.6, 154.2, 135.8, 127.7, 127.4, 126.4, 123.8, 123.5, 122.9, 60.9, 60.8, 42.4, 38.8, 37.6, 34.2, 33.6, 33.5, 33.3, 16.3, 26.1, 25.4.

ES-MS: calcd. for C₂₁H₂₇N₃O₃S (401.18); found (pos.): 402.8 [M+H]; found (neg.): 400.4 [M−H].

### Example 57

### N-{(R)-2-Cyclopentylmethyl-3-oxo-3-[(S)-2-(1H-tetrazol-5-yl)-pyrrolidine-1-yl]-propyl}-N-hydroxy-formamide

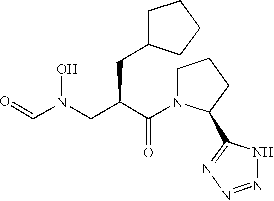

The title compound is prepared from (R)-2-[(Benzyloxy-formyl-amino)-methyl]-3-cyclopentyl-propionic acid A-7 (preparation is described in General Procedure A) and (S)-5-Pyrrolidin-2-yl-1H-tetrazole H-2 (preparation is described in General Procedure H) according to General Procedure B.

1H-NMR (MeOH-d₄, rotamers): δ 8.27 (s, 0.28H), 8.04-7.98 (m, 0.11H), 7.82 (s, 0.62H), 7.77 (s, 0.09H), 5.42-5.30 (m, 1.0H), 3.92-3.75 (m, 2.06H), 3.75-3.66 (m, 1.13H), 3.66-3.52 (m, 0.87H), 3.50-3.32 (m, 0.88H), 3.14-2.98 (m, 0.41H), 2.52-2.18 (m, 2.28H), 2.18-1.92 (m, 2.35H), 1.92-1.73 (m, 1.45H), 1.73-1.43 (m, 7.90H), 1.41-1.25 (m, 1.29H), 1.23-0.85 (m, 2.57H).

13C-NMR (MeOH-d₄, rotamers): δ174.1, 173.7, 163.6, 159.3, 158.3, 157.3, 138.5, 51.8, 50.4, 46.9, 45.8, 41.6, 40.3, 30.6, 24.7, 24.3, 23.5, 23.0.

ES-MS: calcd. for C₁₅H₂₄N₆O₃ (336.19); found (pos.): 337.6 [M+H]; found (neg.): 335.4 [M−H].

### Example 58

### N-Hydroxy-N-{(R)-2-[(S)-2-(1H-tetrazol-5-yl)-pyrrolidine-1-carbonyl]-heptyl}-formamide

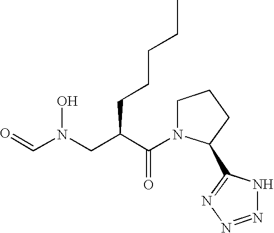

The title compound is prepared from (R)-2-[(Benzyloxy-formyl-amino)-methyl]-hexanoic acid A-7 (preparation is described in General Procedure A) and (S)-5-Pyrrolidin-2-yl-1H-tetrazole H-2 (preparation is described in General Procedure H) according to General Procedure B.

1H-NMR (MeOH-d₄, rotamers): δ 8.27 (s, 0.33H), 7.83 (s, 0.67H), 5.46 (s, 0.34H), 5.38-5.25 (m, 0.66H), 3.88-3.75 (m, 1.92H), 3.75-3.65 (m, 1.08H), 3.58-3.35 (m, 0.86H), 3.25-3.14 (m, 0.73H), 3.10-2.98 (m, 0.42H), 2.40-2.18 (m, 2H), 2.18-2.00 (m, 2H), 1.65-1.09 (m, 8H), 0.92-0.73 (m, 3H).

13C-NMR (MeOH-d₄, rotamers): δ 175.0, 165.0, 163.0, 160.7, 158.8, 53.3, 47.1, 43.5, 42.2, 33.0, 32.0, 31.2, 27.5, 25.7, 23.4, 22.2, 14.9, 13.7.

ES-MS: calcd. for C₁₄H₂₄N₆O₃ (324.39); found (pos.): 325.8 [M+H]; found (neg.): 323.6 [M−H], 437.7 [M+CF₃CO₂].

### Example 59

### (R)-3-[(S)-2-(1H-Benzoimidazol-2-yl)-pyrrolidine-1-carbonyl]-heptanoic acid hydroxyamide

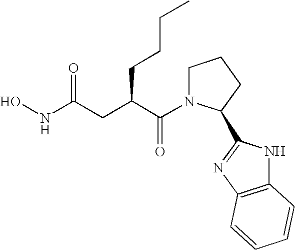

The title compound is prepared from (R)-2-Butyl-succinic acid 4-tert-butyl ester D-1 and (S)-2-Pyrrolidin-2-yl-1H-benzoimidazole E-5 (preparation is described in General Procedure E) according to General Procedure D.

1H-NMR (MeOH-d₄, rotamers): δ 7.50-7.40 (m, 2H), 7.25-7.10 (m, 2H), 5.50 (s, 0.11H), 5.28-5.15 (m, 0.89H), 4.00-3.80 (m, 2H), 3.18-3.02 (m, 1H), 2.40-2.00 (m, 6H), 1.50-1.10 (m, 6H), 0.95-0.74 (m, 3H).

13C-NMR (MeOH-d₄, rotamers): δ 176.7, 171.1, 157.3, 124.3, 122.7, 116.5, 114.9, 57.5, 56.0, 36.0, 32.5, 23.8, 14.9, 13.7.

ES-MS: calcd. for C₁₉H₂₆N₄O₃ (358.44); found (pos.): 359.8 [M+H]; found (neg.): 357.2 [M−H], 471.5 [M+CF₃CO₂].

### Example 60

### (R)-3-((S)-2-Benzooxazol-2-yl-pyrrolidine-1-carbonyl)-heptanoic acid hydroxyamid

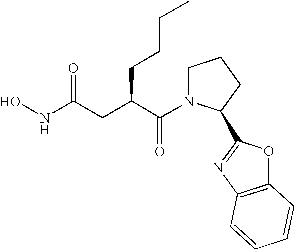

The title compound is prepared from (R)-2-Butyl-succinic acid 4-tert-butyl ester D-1 and (S)-2-Pyrrolidin-2-yl-benzooxazole F-5 (preparation is described in General Procedure F) according to General Procedure D.

1H-NMR (MeOH-d₄, rotamers): δ 7.68-7.57 (m, 1.03H), 7.57-7.45 (m, 0.90H), 7.42-7.27 (m, 2.07H), 5.55 (s, 0.84H), 5.30-5.16 (m, 1.16H), 4.05-3.80 (m, 2H), 3.20-3.10 (m, 1H), 2.50-2.05 (m, 6H), 1.65-1.20 (m, 6H), 1.00-0.80 (m, 3H).

13C-NMR (MeOH-d₄, rotamers): 176.5, 170.9, 168.4, 151.8, 141.9, 126.3, 125.7, 120.4, 111.6, 56.4, 54.8, 41.1, 36.3, 33.6, 31.9, 30.1, 25.7, 23.9, 14.4.

ES-MS: calcd. for C₁₉H₂₅N₃O₄ (359.43); found (pos.) 360.8 [M+H]; found (neg.): 358.6 [M−H], 472.3 [M+CF₃CO₂].

### Example 61

### (R)-3-[(S)-2-(1H-Benzoimidazol-2-yl)-pyrrolidine-1-carbonyl]-heptanoic acid

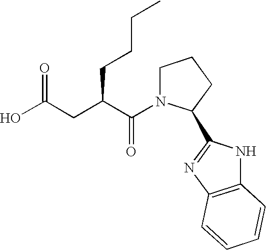

The title compound is prepared from (R)-2-Butyl-succinic acid 4-tert-butyl ester D-1 (as described in D) and (S)-2-Pyrrolidin-2-yl-1H-benzoimidazole E-5 (preparation is described in General Procedure E) according to General Procedure D.

1H-NMR (MeOH-d₄, rotamers): δ 7.80-7.65 (m, 2H), 7.60-7.48 (m, 2H), 5.30-5.18 (m, 1H), 4.15-4.00 (m, 1H), 3.96-3.80 (m, 1H), 3.10-3.00 (m, 1H), 2.68-2.54 (m, 2H), 2.35-2.14 (m, 2H), 2.04-1.98 (m, 2H), 1.45-1.15 (m, 6H), 0.96-0.77 (m, 3H).

13C-NMR (MeOH-d₄, rotamers): δ 177.3, 176.1, 157.3, 139.1, 124.6, 124.5, 124.3, 122.8, 116.5, 114.7, 57.4, 55.9, 41.6, 40.2, 37.2, 33.1, 32.4, 30.1, 25.9, 24.6, 23.8, 14.9, 13.6.

ES-MS: Calcd. for C₁₉H₂₅N₃O₃ (343.43); found (pos.): 344.7 [M+H]; found (neg.): 342.6 [M−H], 456.6 [M+CF₃CO₂].

### Example 62

### (R)-3-((S)-2-Benzooxazol-2-yl-pyrrolidine-1-carbonyl)-heptanoic acid

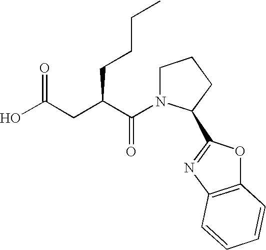

The title compound is prepared from (R)-2-Butyl-succinic acid 4-tert-butyl ester D-1 (as described in D) and (S)-2-Pyrrolidin-2-yl-benzooxazole F-5 (preparation is described in General Procedure F) according to General Procedure D.

1H-NMR (MeOH-d₄, rotamers): δ 7.72-7.58 (m, 1.02H), 7.58-7.45 (m, 0.91H), 7.40-7.20 (m, 2.07H), 5.47 (s, 0.09H), 5.40-5.35 (d, 0.09H), 5.28-5.15 (m, 0.82H). 4.00-3.90 (m, 1H), 3.90-3.78 (m, 1H), 3.18-3.00 (m, 1H), 2.72-2.58 (m, 0.91H), 2.50-2.30 (m, 2.07H), 2.30-2.00 (m, 3.02H), 1.68-1.19 (m, 6H), 0.95-0.80 (m, 3H).

13C-NMR (MeOH-d₄, rotamers): δ 177.0, 175.9, 168.5, 151.8, 142.0, 126.3, 125.7, 120.5, 111.6, 55.4, 40.8, 37.5, 33.5, 31.9, 30.0, 25.8, 23.9, 14.4.

ES-MS: calcd. for C₁₉H₂₄N₂O₄ (344.41); found (pos.): 345.9 [M+H]; found (neg.): 343.6 [M−H], 457.4 [M+CF₃CO₂].

### Example 63

### (R)-3-((S)-2-Benzooxazol-2-yl-pyrrolidine-1-carbonyl)-heptanoic acid methoxy-methyl-amide

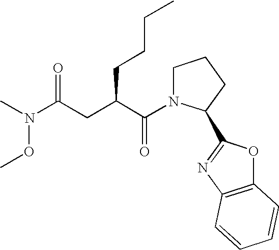

The title compound is prepared from (R)-3-((S)-2-Benzooxazol-2-yl-pyrrolidine-1-carbonyl)-heptanoic acid (preparation is described in example 61) and commercially available O,N-Dimethyl-hydroxylamine by treatment with EDC/HOBt in DMF.

1H-NMR (MeOH-d₄, rotamers): δ 7.60-7.52 (m, 1.02H), 7.52-7.40 (m, 0.89H), 7.37-7.20 (m, 2.09H), 5.48 (s, 0.33H), 5.25-5.10 (m, 0.67H), 4.05-3.92 (m, 1H), 3.92-3.80 (m, 1H), 3.75-3.58 (m, 3H), 3.20-3.00 (m, 4H), 2.95-2.80 (m, 1H), 2.60-2.45 (m, 1H), 2.45-2.30 (m, 1H), 2.30-2.00 (m, 3H), 1.68-1.54 (m, 0.98H), 1.54-1.20 (m, 5.02H), 1.00-0.80 (m, 3H).

13C-NMR (MeOH-d₄, rotamers): δ 177.3, 168.5, 151.8, 142.0, 126.3, 125.7, 120.4, 111.6, 61.7, 56.4, 40.4, 35.9, 33.6, 31.9, 30.1, 25.8, 23.9, 14.4.

ES-MS: calcd. for C₂₁H₂₉N₃O₄ (387.48); found (pos.): 388.9 [M+H].

### Example 64

### (R)-3-[(S)-2-(1H-Benzoimidazol-2-yl)-pyrrolidine-1-carbonyl]-heptanoic acid amide

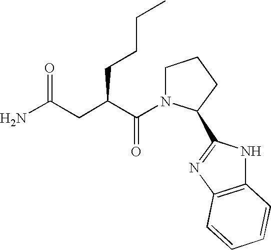

The title compound is prepared by treatment of (R)-3-[(S)-2-(1H-Benzoimidazol-2-yl)-pyrrolidine-1-carbonyl]-heptanoic acid (preparation is described in example 60) with ammonia in methanol.

1H-NMR (MeOH-d₄, rotamers): δ 7.67-7.50 (m, 2H), 7.40-7.30 (m, 2H), 5.50-5.40 (m, 0.21H), 5.27-5.15 (m, 0.79H), 4.03-3.95 (m, 1.02H), 3.95-3.78 (m, 1.06H), 3.68-3.57 (m, 0.44H), 3.15-3.00 (m, 1.12H), 3.00-2.85 (m, 0.28H), 2.65-2.06 (m, 7.37H), 1.48-1.10 (m, 6H), 0.92-0.75 (m, 3H).

13C-NMR (MeOH-d₄, rotamers): δ 176.9, 163.7, 156.8, 150.5, 135.7, 126.4, 124.8, 116.1, 114.5, 56.8, 55.4, 41.7, 38.3, 33.0, 32.5, 31.5, 30.0, 14.9.

ES-MS: calcd. for C₁₉H₂₆N₄O₂ (342.44); found (pos.): 343.9 [M+H]; found (neg.): 341.4 [M−H], 455.5 [M+CF₃CO₂].

### Example 65

### (R)-3-((S)-2-Benzooxazol-2-yl-pyrrolidine-1-carbonyl)-heptanoic acid amide

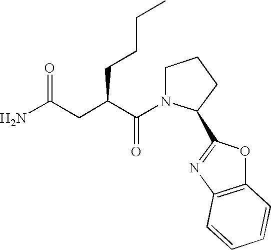

The title compound is prepared by treatment of (R)-3-((S)-2-Benzooxazol-2-yl-pyrrolidine-1-carbonyl)-heptanoic acid (preparation is described in example 61) with ammonia in methanol.

1H-NMR (MeOH-d₄, rotamers): δ 7.65-7.55 (m, 1.05H), 7.55-7.45 (m, 0.91H), 7.40-7.24 (m, 2.03H), 5.30-5.12 (m, 1H), 4.02-3.90 (m, 1.10H), 3.90-3.80 (m, 1.19H), 3.20-3.03 (m, 1.22H), 2.62-2.48 (m, 1.21H), 2.48-2.25 (m, 2.55H), 2.25-2.02 (m, 3.74H), 1.60-1.18 (m, 6H), 0.95-0.75 (m, 3H).

13C-NMR (MeOH-d₄, rotamers): δ 176.8, 168.5, 142.0, 127.1, 126.6, 126.5, 125.4, 124.9, 121.3, 119.7, 112.4, 110.8, 57.2, 55.7, 41.7, 40.4, 38.7, 33.5, 31.9, 25.8, 23.9, 15.0, 13.8.

ES-MS: calcd. for C₁₉H₂₅N₃O₃ (343.43); found (pos.): 344.8 [M+H].

### Example 66

### N-Hydroxy-N-{(R)-2-[(S)-2-(1H-perimidin-2-yl)-pyrrolidine-1-carbonyl]-hexyl}-formamide

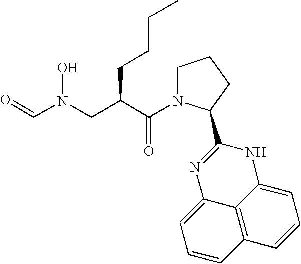

The title compound is prepared from (R)-2-[(Benzyloxy-formyl-amino)-methyl]-hexanoic acid A-7 (preparation is described in General Procedure A) and (S)-2-Pyrrolidin-2-yl-1H-perimidine L-3 (preparation is described in General Procedure L) according to General Procedure B.

1H-NMR (MeOH-d₄, rotamers): δ 8.53 (s, 0.24H), 8.27 (s, 0.20H), 7.87 (s, 0.13H), 7.82 (s, 0.57H), 7.15-6.96 (m, 4.71H), 6.43 (br.s, 2.31H), 4.49-4.38 (m, 0.99H), 3.90-3.80 (t, J=13.4, 8.1, 1.51H), 3.80-3.65 (m, 1.56H), 3.49-3.40 (m, 0.94H), 2.36-1.90 (m, 4.65H), 1.71-1.23 (m, 5.86H), 0.95-0.84 (m, 2.84H).

13C-NMR (MeOH-d₄, rotamers): δ 175.0, 160.6, 159.9, 159.7, 158.7, 140.0, 137.0, 130.0, 128.4, 123.5, 120.9, 62.1, 60.6, 43.6, 42.3, 31.5, 30.2, 24.0, 22.7, 14.9, 13.7.

ES-MS: calcd. for C₂₃H₂₈N₄O₃ (408.22); found (pos.): 409.9 [M+H]; found (neg.): 407.5 [M−H], 521.5 [M+CF₃CO₂].

### Example 67

### N-{(R)-2-Cyclopentylmethyl-3-oxo-3-[(S)-2-(1H-perimidin-2-yl)-pyrrolidin-1-yl]-propyl}-N-hydroxy-formamide

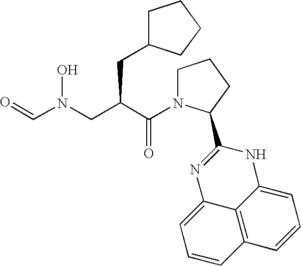

The title compound is prepared from (R)-2-[(Benzyloxy-formyl-amino)-methyl]-3-cyclopentyl-propionic acid A-7 (preparation is described in General Procedure A) and (S)-2-Pyrrolidin-2-yl-1H-perimidine L-3 (preparation is described in General Procedure L) according to General Procedure B.

1H-NMR (MeOH-d₄, rotamers): δ 8.27 (s, 0.19H), 8.21 (s, 0.27H), 7.87 (s, 0.11H), 7.82 (s, 0.60H), 7.17-6.97 (m, 3.88H), 6.53 (d, J=7.1, 0.48H), 6.45-6.38 (m, 1.48H), 4.66-4.57 (m, 0.22H), 4.47-4.39 (m, 0.78H), 4.02-3.95 (m, 0.16H), 3.92-3.55 (m, 3.22H), 3.51-3.40 (m, 0.78H), 3.17-3.08 (m, 0.31H), 2.34-1.86 (m, 5.64H), 1.84-1.45 (m, 6.68H), 1.43-1.28 (m, 0.87H), 1.25-1.02 (m, 2.05H).

13C-NMR (MeOH-d₄, rotamers): δ 175.1, 160.6, 159.8, 158.6, 142.0, 137.0, 130.0, 128.4, 123.4, 121.0, 119.4, 109.6, 108.2, 61.9, 60.4, 43.1, 41.8, 39.3, 38.0, 34.4, 31.4, 27.6, 26.3, 25.8, 25.0.

ES-MS: calcd. for C₂₅H₃₀N₄O₃ (434.23); found (pos.): 436.0 [M+H]; found (neg.): 433.1 [M−H], 547.6 [M+CF₃CO₂].

### Example 68

### N-Hydroxy-N-{(R)-2-[(S)-2-(5-phenyl-1H-imidazol-2-yl)-pyrrolidine-1-carbonyl]-hexyl}-formamide

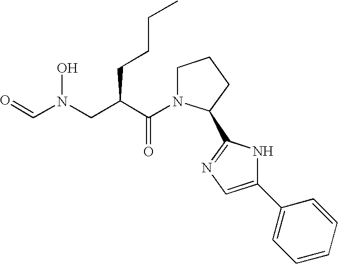

The title compound is prepared from (R)-2-[(Benzyloxy-formyl-amino)-methyl]-hexanoic acid A-7 (preparation is described in General Procedure A) and 5-Phenyl-2-(S)-pyrrolidin-2-yl-1H-imidazole G-2 (preparation is described in General Procedure G) according to General Procedure B.

1H-NMR (MeOH-d₄, rotamers): δ 8.28 (s, 0.16H), 7.84 (s, 0.42H), 7.78 (s, 0.21H), 7.63 (d, J=7.4, 2.08H), 7.40-7.29 (m, 2.45H), 7.29-7.18 (m, 1.67H), 5.30-5.24 (m, 0.41H), 5.15-5.08 (m, 0.59H), 3.93-3.78 (m, 1.35H), 3.78-3.65 (m, 1.32H), 3.65-3.53 (m, 0.70H), 3.48-3.35 (m, 0.75H), 3.25-3.14 (m, 0.50H), 3.14-3.02 (m, 0.27H), 2.93-2.82 (m, 0.22H), 2.67-2.57 (m, 0.24H), 2.45-2.32 (m, 0.47H), 2.32-2.10 (m, 2.26H), 2.10-1.93 (m, 0.45H), 1.93-1.80 (m, 0.45H), 1.66-1.17 (m, 6.47H), 0.96-0.87 (m, 1.36H), 0.87-0.79 (m, 1.82H).

13C-NMR (MeOH-d₄, rotamers): δ 160.7, 158.8, 130.5, 128.9, 126.7, 125.1, 55.5, 50.4, 47.3, 43.8, 32.6, 30.9, 30.2, 14.8, 13.6, 13.0.

ES-MS: calcd. for C₂₁H₂₈N₄O₃ (384.22); found (pos.): 385.6 [M+H]; found (neg.): 383.2 [M−H], 497.3 [M+CF₃CO₂].

### Example 69

### N-{(R)-2-Cyclopentylmethyl-3-oxo-3-[(S)-2-(5-phenyl-1H-imidazol-2-yl)-pyrrolidine-1-yl]-propyl}-N-hydroxy-formamide

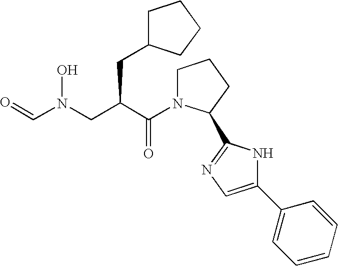

The title compound is prepared from (R)-2-[(Benzyloxy-formyl-amino)-methyl]-3-cyclopentyl-propionic acid A-7 (preparation is described in General Procedure A) and 5-Phenyl-2-(S)-pyrrolidin-2-yl-1H-imidazole G-2 (preparation is described in General Procedure G) according to General Procedure B.

1H-NMR (MeOH-d₄, rotamers): δ 8.27 (s, 0.16H), 8.22 (s, 0.30H), 7.83 (s, 0.39H), 7.77 (s, 0.15H), 7.74-7.60 (m, 2.17H), 7.47 (s, 0.04H), 7.39-7.28 (m, 2.92H), 7.28-7.20 (m, 0.95H), 5.30-5.25 (m, 0.35H), 5.19-5.08 (m, 0.65H), 3.95-3.79 (m, 1.45H), 3.79-3.67 (m, 0.95H), 3.67-3.51 (m, 0.74H), 3.50-3.39 (m, 0.67H), 3.16-3.06 (m, 0.22H), 2.91-2.82 (m, 0.16H), 2.67-2.56 (m, 0.20H), 2.49-2.10 (m, 2.77H), 2.00-1.95 (m, 1.14H), 1.94-1.30 (m, 9.73H), 1.24-0.96 (m, 2.12H).

13C-NMR (MeOH-d₄, rotamers): δ175.2, 167.3, 165.3, 164.9, 164.6, 162.6, 160.6, 158.6, 150.9, 137.8, 133.4, 130.6, 130.5, 128.9, 125.4, 125.1, 118.5, 116.6, 56.8, 55.3, 43.1, 41.8, 39.4, 32.1, 27.4, 26.1, 24.9.

ES-MS: calcd. for C₂₃H₃₀N₄O₃ (410.23); found (pos.): 412.0 [M+H]; found (neg.): 409.6 [M−H], 523.8 [M+CF₃CO₂].

### Example 70

### N-{1-[(S)-2-(1H-Benzoimidazol-2-yl)-pyrrolidine-1-carbonyl]-cyclohexylmethyl}-N-hydroxy-formamide

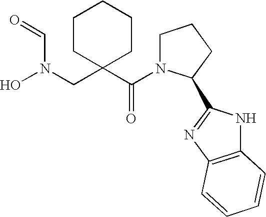

The title compound is prepared from 1-[(Benzyloxy-formyl-amino)-methyl]-cyclohexanecarboxylic acid M-5 (preparation is described in General Procedure M) and (S)-2-Pyrrolidin-2-yl-1H-benzoimidazole E-5 (preparation is described in General Procedure E) according to General Procedure B.

1H-NMR (MeOH-d₄, rotamers): δ 8.39 (s, 0.62H), 7.92 (s, 0.40H), 7.48 (br.s, 1.80H), 7.22-7.14 (m, 2.24H), 5.31 (br.s, 1.0H), 4.28-4.14 (m, 1.11H), 4.07-3.97 (m, 0.68H), 3.90-3.70 (m, 2.76H), 2.37-1.95 (m, 7.10H), 1.67-1.23 (m, 9.58H).

13C-NMR (MeOH-d₄, rotamers): δ 174.9, 168.1, 164.9, 157.8, 124.3, 122.7, 60.1, 58.6, 36.0, 32.3, 31.3, 28.1, 26.8, 25.2, 23.9, 22.8, 5.2.

ES-MS: calcd. for C₂₀H₂₆N₄O₃ (370.20); found (pos.): 371.9 [M+H]; found (neg.): 369.2 [M−H].

### Example 71

### N-{(R)-2-[(S)-2-(1H-Benzoimidazol-2-yl)-piperidine-1-carbonyl]-hexyl}-N-hydroxy-formamide

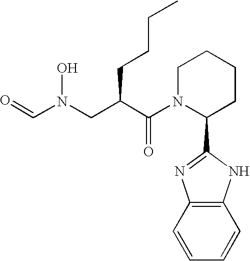

The title compound is prepared from (R)-2-[(Benzyloxy-formyl-amino)-methyl]-hexanoic acid A-7 (preparation is described in General Procedure A) and 5 (S)-2-Piperidin-2-yl-1H-benzoimidazole E-5 (preparation is described in General Procedure E) according to General Procedure B.

1H-NMR (MeOH-d₄, rotamers): δ 8.1-8.0 (m, 0.42H), 7.98-7.84 (m, 0.53H), 7.6-7.44 (bs, 1.83H), 7.30-7.10 (m, 2.20H), 6.1-6.01 (d, J=3.76, 0.46H), 5.80-5.72 (d, J=4.64, 0.24H), 5.72-5.62 (d, J=3.64, 0.29H), 4.75-4.55 (m, 0.70H), 7.34-7.25 (m, 1.93H), 5.39 (d, J=7.8, 0.29H), 5.25-5.05 (m, 0.71H), 3.70-3.40 (m, 1.30H), 3.40-3.30 (m, 1.30H), 3.23-3.03 (m, 0.84H), 2.75-2.60 (m, 0.89H), 2.60-2.30 (m, 0.78H), 1.95-1.80 (m, 1H), 1.80-1.44 (m, 6.36H), 1.44-1.25 (m, 5.84H), 1.00-0.8 (m, 3.39H).

ES-MS: calcd. for C₂₀H₂₈N₄O₃ (372.47); found (pos.): 373.9 [M+H].

### Example 72

### N-{(R)-2-[(R)-4-(1H-Benzoimidazol-2-yl)-thiazolidine-3-carbonyl]-hexyl}-N-hydroxy-formamide

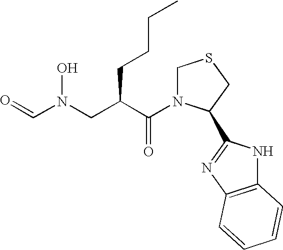

The title compound is prepared from (R)-2-[(Benzyloxy-formyl-amino)-methyl]-hexanoic acid A-7 (preparation is described in General Procedure A) and (R)-2-Thiazolidin-4-yl-1H-benzoimidazole E-5 (preparation is described in General Procedure E) according to General Procedure B.

1H-NMR (MeOH-d4, rotamers): δ 8.52 (s, 0.07H), 8.21 (m, 0.23H), 7.9-7.8 (m, 0.88H), (s, 1.8H), (m, 2.2H), 5.89 (m, 0.72H), 5.66 (m, 0.55H), (m, H), (dd, J=0.04, 0.38H), 3.84-3.7 (m, 1.2H), 3.67-3.45 (m, 3.2H), 3.14-3.1 (m, 0.49H), 1.79-1.25 (m, 8H), 0.97-0.75 (m, 3.6H).

ES-MS: calcd. for C₁₈H₂₄N₄O₃S (376.5); found (pos.): 377.1 [M+H].

### Example 73

### 2-((S)-1-{(R)-2-[(Formyl-hydroxy-amino)-methyl]-hexanoyl}-pyrrolidin-2-yl)-thiazole-4-carboxylic acid amide

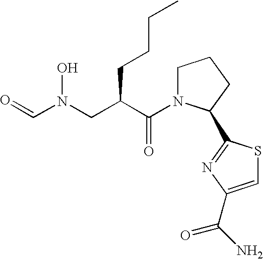

The title compound is prepared from (R)-2-[(Benzyloxy-formyl-amino)-methyl]-hexanoic acid A-7 (preparation is described in General Procedure A) and (S)-2-Pyrrolidin-2-yl-thiazole-4-carboxylic acid amide J-4 (preparation is described in General Procedure J) according to General Procedure C.

1H-NMR (MeOH-d₄, rotamers): δ 8.30-8.15 (m, 0.40H), 8.15-8.0 (m, 0.80H), 7.90-7.75 (m, 0.52H), 7.73 (s, 0.08H), 5.70-5.60 (m, 0.06H), 5.60-5.46 (m, 0.17H), 5.46-5.25 (m, 0.78H), 4.0-3.90 (m, 0.18H), 3.90-3.52 (m, 3.10H), 3.52-3.30 (m, 0.69H), 3.20-3.00 (m, 0.38H), 2.60-2.40 (m, 0.16H), 2.40-2.20 (m, 1.68H), 2.20-1.90 (m, 2.14H), 1.76-1.40 (m, 2.32H), 1.40-1.15 (m, 4.25H), 1.0-0.80 (m, 3H).

ES-MS: calcd. for C₁₆H₂₄N₄O₄S (368.46); found (pos.): 369.20 [M+H].

### Example 74

### 2-((S)-1-{(R)-2-[(Formyl-hydroxy-amino)-methyl]-hexanoyl}-pyrrolidin-2-yl)-thiazole-4-carboxylic acid ethyl ester

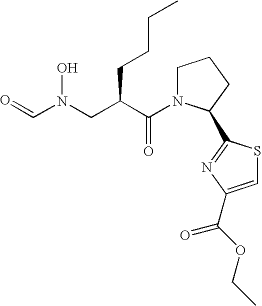

The title compound is prepared from (R)-2-[(Benzyloxy-formyl-amino)-methyl]-hexanoic acid A-7 (preparation is described in General Procedure A) and (S)-2-Pyrrolidin-2-yl-thiazole-4-carboxylic acid ethyl ester J-4 (preparation is described in General Procedure J) according to General Procedure C.

1H-NMR (MeOH-d₄, rotamers): δ 8.27 (s, 1.1H), 7.84 (s, 0.52H), 5.44-5.26 (m, 1H), 4.36 (q, J=7, 2.36H), 3.90-3.60 (m, 3.46H), 3.54-3.34 (m, 0.83H), 3.20-3.00 (m, 0.36), 2.40-2.20 (m, 2.32H), 2.20-2.04 (m, 2.44H), 1.74-1.42 (m, 2.84H), 1.42-1.26 (m, 8.10H), 1.0-0.74 (m, 3.56H).

ES-MS: calcd. for C₁₈H₂₇N₃O₅S (397.50); found (pos.): 398.6 [M+H].

### Example 75

### N-{(R)-2-[(R)-2-(1H-Benzoimidazol-2-yl)-pyrrolidine-1-carbonyl]-hexyl}-N-hydroxy-formamide

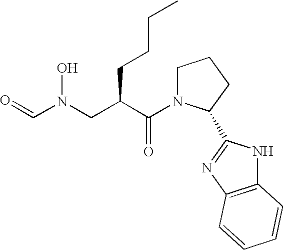

The title compound is prepared as a mixture with N-{(R)-2-[(S)-2-(1H-Benzoimidazol-2-yl)-pyrrolidine-1-carbonyl]-hexyl}-N-hydroxy-formamide (preparation is described in example 1) and isolated through purification by preparative HPLC.

1H-NMR (MeOH-d₄, rotamers): δ 8.64-8.6 (m, 0.04H), 8.35 (s, 0.23H), 8.33-8.25 (m, 0.16H), 8.01-7.95 (m, 0.07H), 7.81 (s, 0.25H), 7.72 (s, 0.26H), 7.64-7.6 (m, 0.06H), 7.61-7.45 (m, 1.7H), 7.51-7.45 (m, 0.07H), 7.29-7.15 (m, 1.8H), 5.58-5.5 (m, 0.29H), 5.44-5.5 (m, 0.12H), 5.43-5.35 (m, 0.27H), 5.35-5.25 (m, 0.33H), 4.12-4.0 (m, 0.32H), 3.98-3.8 (m, 1H), 3.62-3.8 (m, 1.5H), 3.62-3.5 (m, 0.23H), 3.5-3.46 (m, 0.18H), 3.44-3.4 (m, 0.18H), 3.4-3.36 (m, 0.15H), 3.25-3.15 (m, 0.44H), 3.16-3.1 (m, 0.07H), 3.07-2.95 (m, 0.5H), 2.85 (s, 0.2H), 2.80 (s, 0.6H), 2.61-2.45 (m, 0.47H), 2.45-2.31 (m, 0.6H), 2.29-1.95 (m, 3H), 1.73-1.55 (m, 0.76H), 1.55-1.43 (m, 0.65H), 1.45-1.19 (m, 4H), 1.25-1.05 (m, 1.4H), 0.99-0.85 (m, 1.88H), 0.82-0.6 (m, 1.14H), 0.45-0.29 (m, 1.4H).

ES-MS: calcd. for C₁₉H₂₆N₄O₃ (358.4); found (pos.): 359.6 [M+H].

### Example 76

### N—[(R)-2-((R)-2-Benzooxazol-2-yl-pyrrolidine-1-carbonyl)-hexyl]-N-hydroxy-formamide

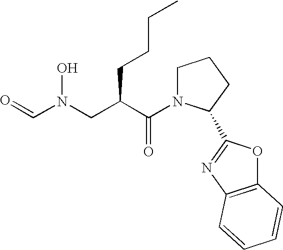

The title compound is prepared as a mixture with N—[(R)-2-((R)-2-Benzooxazol-2-yl-pyrrolidine-1-carbonyl)-hexyl]-N-hydroxy-formamide (preparation is described in example 38) and isolated through purification by preparative HPLC.

1H-NMR (CDCl₃, rotamers): δ 8.50 (s, 0.74H), 8.37-8.20 (m, 0.09H), 7.87-7.80 (m, 0.30H), 7.51 (s, 0.21H), 7.60-7.42 (1.83H), 7.38-7.22 (m, 2.18H), 5.50-5.38 (m, 1.03H), 5.28 (s, 0.12H), 4.18-4.07 (dd, J=13.9, 4.5, 0.79H), 4.07-4.00 (m, 0.23H), 3.87-3.78 (m, 1.12H), 3.77-3.40 (m, 2.84H), 3.32-3.22 (m, 0.28H), 3.18-3.07 (m, 0.84H), 2.44-2.18 (m, 2.71H), 2.14-1.88 (m, 2.64H), 1.87-1.80 (m, 0.25), 1.78-1.60 (m, 1.87H), 1.52-1.20 (m, 6.10H), 0.98-0.85 (m, 3.3H), 0.43-0.37 (t, 0.17H).

13C-NMR (CDCl₃, rotamers): δ 173.49, 166.27, 163.08, 156.80, 150.83, 139.06, 125.58, 125.14, 124.98, 124.70, 120.34, 119.10, 110.90, 110.65, 54.62, 54.46, 52.48, 51.53, 47.45, 47.19, 42.57, 41.58, 30.50, 30.15, 29.68, 29.60, 29.55, 24.25, 24.14, 22.78, 22.74, 13.90.

ESI-MS: calcd. for C19H25N3O4 (359.18); found (pos.): 360.5 [M+H]. found (neg.): 358.4[M−H].

### Example 77

### N—[(R)-3-((R)-2-Benzothiazol-2-yl-pyrrolidine-1-yl)-2-cyclopentylmethyl-3-oxo-propyl]-N-hydroxy-formamide

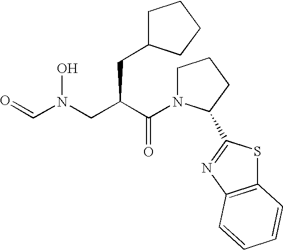

The title compound is prepared as a mixture with N—[(R)-3-((R)-2-Benzothiazol-2-yl-pyrrolidine-1-yl)-2-cyclopentylmethyl-3-oxo-propyl]-N-hydroxy-formamide (preparation is described in example 53) and isolated through purification by preparative HPLC.

1H-NMR (MeOH-d₄, rotamers): δ 8.19 (s, 0.24H), 7.74 (s, 0.58H), 7.69 (s, 0.07H), 7.65-7.58 (m, 0.17H), 7.55-7.47 (m, 0.94H), 7.45-7.38 (m, 0.84H), 7.35-7.22 (m, 1.97H), 5.40-5.30 (m, 0.15H), 5.18-5.11 (m, 0.85H), 3.87-3.71 (m, 1.76H), 3.71-3.47 (m, 1.56H), 3.42-3.32 (m, 0.78H), 3.27-3.21 (m, 1.17H), 3.12-2.99 (m, 0.36H), 2.44-2.26 (m, 1.13H), 2.18-1.90 (m, 2.96H), 1.86-1.49 (m, 2.09H), 1.49-1.24 (m, 6.16H), 1.14-0.89 (m, 2.12H).

13C-NMR (MeOH-d₄, rotamers): δ 175.2, 168.1, 159.6, 151.8, 142.0, 126.4, 125.8, 120.5, 111.6, 99.5, 56.5, 53.8, 42.5, 38.8, 37.4, 34.2, 33.7, 31.8, 26.2, 25.7.

ES-MS: calcd. for C₂₁H₂₇N₃O₃S (401.18); found (pos.): 402.4 [M+H]; found (neg.): 400.3 [M−H].

### Example 78

### N-Hydroxy-N-{(R)-2-[(S)-2-(5-nitro-1H-benzoimidazol-2-yl)-pyrrolidine-1-carbonyl]-hexyl}-formamide

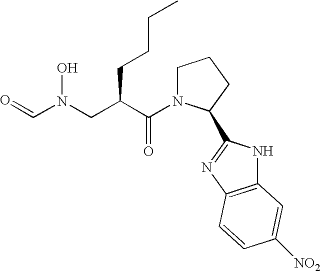

The title compound is prepared from (R)-2-[(Benzyloxy-formyl-amino)-methyl]-hexanoic acid A-7 (R1=n-butyl, preparation is described in General Procedure A) and 5-Nitro-2-(S)-pyrrolidin-2-yl-1H-benzoimidazole E-5 (preparation is described in General Procedure E; PG=Boc) according to General Procedure C.

1H-NMR (MeOH-d₄, rotamers): δ 8.43 (s, 0.67H), 8.27 (s, 0.20H), 8.25-8.10 (m, 0.94H), 7.85-7.55 (m, 1.6H), 5.52-5.35 (m, 0.21H), 5.30-5.13 (m, 0.71H), 4.0-3.83 (m, 1.5H), 3.83-3.55 (m, 1.71H), 3.55-3.33 (m, 0.70H), 2.50-2.0 (m, 3.73H), 1.65-1.10 (m, 6.5H), 1.0-0.72 (m, 3.13H). ES-MS: calcd. for C₁₉H₂₅N₅O₅ (403.44); found (pos.): 404.7 [M+H].

### Example 79

### N-Hydroxy-N-{(R)-2-[(S)-2-(5-trifluoromethoxy-1H-benzoimidazol-2-yl)-pyrrolidine-1-carbonyl]-hexyl}-formamide

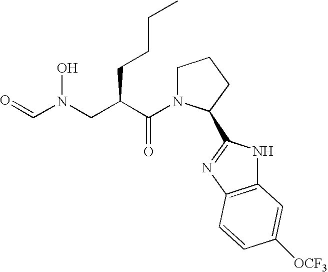

The title compound is prepared from (R)-2-[(Benzyloxy-formyl-amino)-methyl]-hexanoic acid A-7 (R2=n-butyl, preparation is described in General Procedure A) and 5-Trifluoromethoxy-2-(S)-pyrrolidin-2-yl-1H-benzoimidazole E-5 (preparation is described in General Procedure E; PG=Cbz) according to General Procedure B. 1H-NMR (MeOH-d₄, rotamers): δ 8.27 (s, 0.20H), 7.9-7.74 (m, 0.70H), 7.69-7.50 (m, 1.10H), 7.50-7.36 (m, 0.83H), 7.25-7.06 (m, 0.99H), 5.50-5.34 (m, 0.29H), 5.28-5.08 (m, 0.75H), 3.86-3.53 (m, 3.24H), 3.53-3.33 (m, 0.85H), 2.60-2.0 (m, 4.18H), 1.64-1.10 (m, 6.4H), 1.0-0.70 (m, 3.0H). ES-MS: calcd. for C₂₀H₂₅F₃N₄O₄ (442.44); found (pos.): 443.4 [M+H].

### Example 80

### 2-((S)-1-{(R)-2-[(Formyl-hydroxy-amino)-methyl]-hexanoyl}-pyrrolidin-2-yl)-1H-benzoimidazol-5-sulfonic acid amide

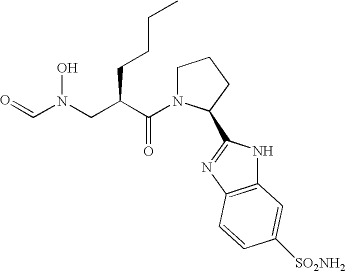

The title compound is prepared from (R)-2-[(Benzyloxy-formyl-amino)-methyl]-hexanoic acid A-7 (R2=n-butyl, preparation is described in General Procedure A) and (S)-2-Pyrrolidin-2-yl-3H-benzoimidazole-5-sulfonic acid amide E-5 (preparation is described in General Procedure E; PG=Cbz) according to General Procedure B.

1H-NMR (MeOH-d₄, rotamers): δ 8.27 (s, 0.20H), 8.13 (s, 0.23H), 8.10 (s, 0.61H), 7.9-7.74 (m, 1.70H), 7.74-7.66 (m, 0.24H), 7.63 (d, J=8.5 Hz, 0.72H), 5.50-5.30 (m, 0.279H), 5.30-5.10 (m, 0.78H), 4.0-3.80 (m, 1.6H), 3.80-3.60 (m, 1.73H), 3.55-3.35 (m, 0.82H), 3.20-3.05 (m, 0.34H), 2.95-2.80 (m, 0.26H), 2.75-2.60 (m, 0.16H), 2.60-2.45 (m, 0.29H), 2.45-2.23 (m, 1.79H), 2.23-1.97 (m, 2.59H), 1.97-1.80 (m, 0.34H), 1.66-1.49 (m, 1.19H), 1.49-1.38 (m, 1.1H), 1.38-1.12 (m, 4.25H), 1.0-0.89 (m, 0.84H), 0.89-0.72 (m, 2.28H). ES-MS: calcd. for C₁₉H₂₇N₅O₅S (437.52); found (pos.): 438.6 [M+H].

### Example 81

### N-{(R)-2-[(2S,4R)-2-(6,7-Dihydro-1H-5,8-dioxa-1,3-diaza-cyclopenta[b]naphthalene-2-yl)-4-fluoro-pyrrolidine-1-carbonyl]-hexyl}-N-hydroxy-formamide

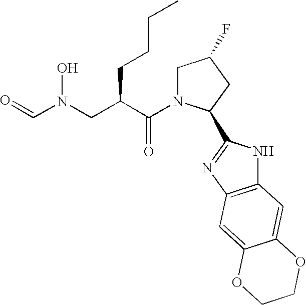

The title compound is prepared from (R)-2-[(Benzyloxy-formyl-amino)-methyl]-hexanoic acid A-7 (R2=n-butyl, preparation is described in General Procedure A) and 2-((2S,4R)-4-Fluoro-pyrrolidin-2-yl)-6,7-dihydro-1H-5,8-dioxa-1,3-diaza-cyclopenta[b]naphthalene E-5 (preparation is described in General Procedure E; PG=Boc) according to General Procedure B.

1H-NMR (MeOH-d₄, rotamers): δ 8.23 (s, 0.27H), 8.14 (s, 0.095H), 7.82 (s, 0.41H), 7.18 (s, 0.15H), 7.67 (s, 0.15H), 7.00 (s, 0.27H), 6.95 (s, 0.30H), 6.93 (s, 1.15H), 5.49 (s, 0.40H), 5.46-5.31 (m, 0.92H), 5.31-5.17 (m, 0.96H), 4.23 (s, 4H), 4.15-3.92 (m, 1.05H), 3.84-3.56 (m, 1.37H), 3.56-3.33 (m, 1.17H), 3.22-3.10 (m, 0.54H), 3.10-2.96 (m, 0.34H), 2.96-2.76 (m, 0.55H), 2.76-2.60 (m, 0.97H), 2.60-2.49 (m, 0.44H), 2.49-2.30 (m, 0.58H), 1.6-1.24 (m, 3.67H), 1.24-1.02 (m, 3.1H), 0.96-0.83 (m, 1H), 0.83-0.66 (m, 2.2H). ES-MS: calcd. for C₂₁H₂₇FN₄O₅ (434.47); found (pos.): 435.6 [M+H].

### Example 82

### N-{(R)-2-Cyclopentylmethyl-3-[(2S,4R)-2-(6,7-Dihydro-1H-5,8-dioxa-1,3-diaza-cyclopenta[b]naphthalene-2-yl)-4-fluoro-pyrrolidin-1-yl]-3-oxo-propyl}-N-hydroxy-formamide

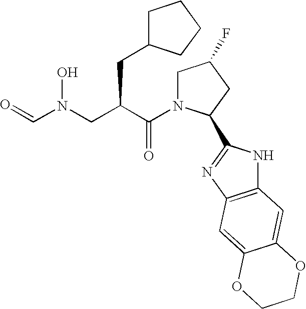

The title compound is prepared from (R)-2-[(Benzyloxy-formyl-amino)-methyl]-3-cyclopentyl-propionic acid A-7 (R2=cyclopentylmethyl, preparation is described in General Procedure A) and 2-((2S,4R)-4-Fluoro-pyrrolidin-2-yl)-6,7-dihydro-1H-5,8-dioxa-1,3-diaza-cyclopenta[b]naphthalene E-5 (preparation is described in General Procedure E; PG=Boc) according to General Procedure B.

1H-NMR (MeOH-d₄, rotamers): δ 8.22 (s, 0.27H), 8.11 (s, 0.22H), 7.82 (s, 0.38H), 7.72 (s, 0.12H), 7.64 (s, 0.12H), 7.0 (s, 0.2H), 6.97 (s, 0.2H), 6.93 (s, 1.05H), 5.50 (s, 0.38H), 5.44-5.32 (m, 0.78H), 5.32-5.16 (m, 0.84H), 4.23 (s, 4.6H), 4.15-3.88 (m, 0.76H), 3.88-3.65 (m, 1.16H), 3.50-3.34 (m, 1.12H), 3.25-2.85 (m, 0.98H), 2.80-2.53 (m, 1.24H), 2.53-2.40 (m, 0.5H), 2.40-2.30 (m, 0.14H), 1.90-1.70 (m, 1.73H), 1.70-1.40 (m, 6.1H), 1.40-1.28 (m, 1.41H), 1.28-1.14 (m, 0.28H), 1.14-0.97 (m, 1.03H), 0.97-0.80 (m, 0.69H). ES-MS: calcd. for C₂₃H₂₉FN₄O₅ (460.51); found (pos.): 461.4 [M+H].

### Example 83

### N-{2Cyclopentylmethyl-3-[2-(6,7-dihydro-1H-5,8-dioxa-1,3-diaza-cyclopenta[b]naphthalen-2-yl)-pyrrolidin-1-yl]-3-oxo-propyl}-N-hydroxy-formamide

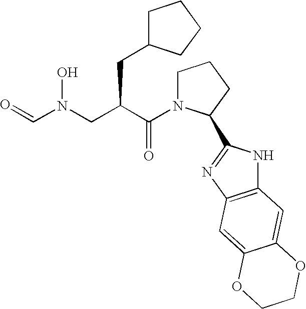

The title compound is prepared from (R)-2-[(Benzyloxy-formyl-amino)-methyl]-3-cyclopentyl-propionic acid A-7 (R2=cyclopentylmethyl, preparation is described in General Procedure A) and (S)-2-Pyrrolidin-2-yl-6,7-dihydro-1H-5,8-dioxa-1,3-diaza-cyclopenta[b]naphthalene E-5 (preparation is described in General Procedure E; PG=Cbz) according to General Procedure B.

1H-NMR (MeOH-d₄, rotamers): δ 8.27 (s, 0.15H), 8.21 (s, 0.19H), 7.86 (s, 0.17H), 7.83 (s, 0.37H), 7.75 (s, 0.16H), 7.03 (s, 0.24H), 6.98 (s, 0.30H), 6.93 (s, 0.90H), 5.33 (d, J=6.9, 0.40H), 5.19-5.11 (m, 0.60H), 4.23 (s, 4.0H), 3.93-3.79 (m, 1.48H), 3.76-3.65 (m, 1.20H), 3.65-3.55 (m, 0.71H), 3.48-3.35 (m, 0.98H), 3.15-3.05 (m, 0.29H), 2.95-2.84 (m, 0.23H), 2.67-2.59 (m, 0.24H), 2.49-2.37 (m, 0.48H), 2.37-2.11 (m, 2.47H), 2.11-1.97 (m, 1.18H), 1.87-1.31 (m, 9.90H), 1.23-0.93 (m, 2.24H). ¹³C-NMR (MeOH-d₄, rotamers): δ 176.3, 175.7, 175.2, 166.2, 163.9, 163.4, 159.9, 159.6, 156.3, 155.1, 142.9, 142.8, 142.5, 102.5, 65.5, 57.6, 56.6, 53.4, 38.9, 33.9, 33.8, 32.1, 26.3, 26.2, 26.1, 25.9, 23.2. ES-MS: calcd. for C₂₃H₃₀N₄O₅ (442.22); found (pos.): 443.6 [M+H]; found (neg.): 441.7 [M−H].

### Example 84

### N-{2-[2-(7,8-Dihydro-3H-6,9-dioxa-1,3-diaza-cyclopenta[α]naphthalen-2-yl)-pyrrolidine-1-carbonyl]-hexyl}-N-hydroxy-formamide

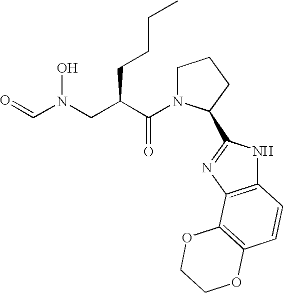

The title compound is prepared from (R)-2-[(Benzyloxy-formyl-amino)-methyl]-hexanoic acid A-7 (R2=n-butyl, preparation is described in General Procedure A) and (S)-2-Pyrrolidin-2-yl-7,8-dihydro-3H-6,9-dioxa-1,3-diaza-cyclopenta[a]naphthalene E-5 (preparation is described in General Procedure E; PG=Cbz) according to General Procedure B.

1H-NMR (MeOH-d₄, rotamers): δ 8.27 (s, 0.16H), 8.15 (s, 0.18H), 7.94 (s, 0.20H), 7.83 (s, 0.38H), 7.72 (s, 0.12H), 7.10-7.05 (m, 0.12H), 7.01-6.93 (m, 0.79H), 6.73-6.62 (m, 0.91H), 5.39-5.31 (m, 0.39H), 5.21-5.13 (m, 0.61H), 4.37-4.22 (m, 4.0H), 3.96-3.78 (m, 1.58H), 3.78-3.55 (m, 1.81H), 3.48-3.37 (m, 0.92H), 3.27-3.16 (m, 0.54H), 3.12-3.03 (m, 0.30H), 2.95-2.81 (m, 0.25H), 2.63-2.56 (m, 0.25H), 2.48-2.13 (m, 2.74H), 2.11-1.95 (m, 1.15H), 1.91-1.72 (m, 0.40H), 1.63-1.14 (m, 6.64H), 0.95-0.79 (m, 3.07H). ¹³C-NMR (MeOH-d₄, rotamers): δ 176.4, 175.5, 175.1, 165.6, 164.0, 163.7, 159.9, 159.7, 156.2, 155.3, 140.0, 133.0, 115.0, 114.6, 114.3, 107.8, 66.0, 66.0, 65.7, 56.7, 53.2, 43.0, 32.4, 30.9, 30.2, 30.1, 25.8, 24.0, 23.8, 14.2. ES-MS: calcd. for C₂₁H₂₈N₄O₅ (416.21); found (pos.): 417.1 [M+H]; found (neg.): 415.5 [M−H].

### Example 85

### N-Hydroxy-N-{2-[2-(3-methyl-3H-imidazo[4,5-c]pyridin-2-yl]-pyrrolidine-1-carbonyl}-hexyl)-formamide

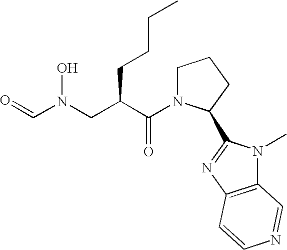

The title compound is prepared from (R)-2-[(Benzyloxy-formyl-amino)-methyl]-hexanoic acid A-7 (R2=n-butyl, preparation is described in General Procedure A) and 3-Methyl-2-(S)-pyrrolidin-2-yl-3H-imidazo[4,5-c]pyridine N-3 (preparation is described in General Procedure N; PG=Cbz) according to General Procedure B.

1H-NMR (CDCl₃, rotamers): δ 8.92 (s, 0.65H), 8.62-8.22 (m, 4.95H), 8.10 (s, 0.37H), 7.87 (d, J=6.7, 0.70H), 7.80-7.67 (m, 2.14H), 5.38-5.30 (m, 1H), 4.35-4.20 (m, 3.33H), 4.09-3.90 (m, 1.30H), 3.76-3.53 (m, 2.63H), 3.38-3.30 (m, 0.70H), 2.84-2.72 (m, 1.36H), 2.66-2.58 (m, 0.39H), 2.52-2.33 (m, 1.87H), 2.00-1.86 (m, 1.12H), 1.80-1.52 (m, 2.30H), 1.52-1.15 (m, 6.49H), 0.94-0.74 (m, 3.70H).

13C-NMR (CDCl₃, rotamers): δ 175.2, 174.3, 173.8, 173.5, 165.1, 161.5, 157.5, 154.0, 143.4, 142.6, 133.3, 131.9, 131.6, 113.7, 113.3, 58.1, 57.8, 53.1, 48.0, 47.0, 46.8, 46.5, 46.3, 43.8, 43.6, 33.8, 32.8, 31.6, 31.4, 29.2, 29.1, 22.9, 22.8, 22.4, 13.9, 13.8.

ES-MS: calcd. for C₁₉H₂₇N₅O₃ (373.21); found (pos.): 374.3 [M+H].

### Example 86

### N-Hydroxy-N-(2-{2-[3-(2-hydroxy-ethyl)-3H-imidazo[4,5-c]pyridin-2-yl]-pyrrolidine-1-carbonyl}-hexyl)-formamide

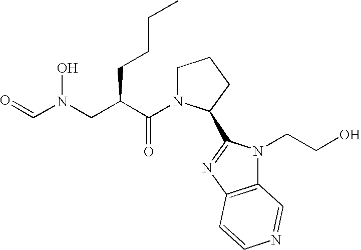

The title compound is prepared from (R)-2-[(Benzyloxy-formyl-amino)-methyl]-hexanoic acid A-7 (R2=n-butyl, preparation is described in General Procedure A) and 2-((S)-2-Pyrrolidin-2-yl-imidazo[4,5-c]pyridin-3-yl)-ethanol N-3 (preparation is described in General Procedure N; PG=Cbz) according to General Procedure B.

1H-NMR (CDCl₃, rotamers): δ 9.07 (s, 0.37H), 8.95-8.90 (m, 0.59H), 8.42 (d, J=6.3, 0.39H), 8.36 (s, 0.45H), 8.28 (s, 0.13H), 8.22-8.13 (m, 0.57H), 8.00 (s, 0.18H), 7.94 (d, J=6.7, 0.39H), 7.87-7.78 (m, 0.86H), 7.74 (s, 0.36H), 5.46-5.40 (m, 0.57H), 5.34-5.25 (m, 0.43H), 4.69-4.53 (m, 2.01H), 4.03-3.82 (m, 319H), 3.82-3.60 (m, 2.25H), 3.52-3.41 (m, 0.89H), 318-3.07 (m, 0.17H), 2.98-2.80 (m, 0.40H), 2.68-2.59 (m, 0.58H), 2.53-2.36 (m, 1.27H), 2.39-2.24 (m, 0.44H), 2.19-2.06 (m, 0.89H), 2.06-1.95 (m, 0.64H), 1.87-1.72 (m, 0.62H), 1.64-1.24 (m, 6.38H), 0.96-0.75 (m, 3.13H).

13C-NMR (CDCl₃, rotamers): δ 176.1, 175.9, 175.0, 174.2, 167.9, 163.5, 159.7, 155.1, 143.7, 136.9, 135.8, 134.7, 134.6, 134.4, 134.2, 113.7, 113.5, 112.5, 63.4, 62.3, 62.1, 59.6, 57.6, 53.8, 47.7, 44.6, 42.9, 34.2, 32.6, 32.1, 30.1, 24.0, 23.9, 23.8, 14.3, 14.2.

ES-MS: calcd. for C₂₀H₂₉N₅O₄ (403.22); found (pos.): 404.7 [M+H]; found (neg.): 402.6.

### Example 87

### N-Hydroxy-N-(2-{2-[3-(2-methoxy-ethyl)-3H-imidazo[4,5-c]pyridin-2-yl]-pyrrolidine-1-carbonyl}-hexyl)-formamide

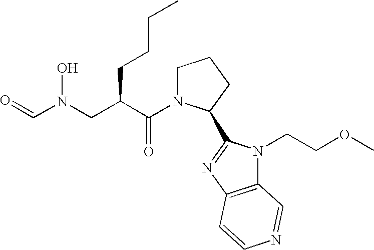

The title compound is prepared from (R)-2-[(Benzyloxy-formyl-amino)-methyl]-hexanoic acid A-7 (R2=n-butyl, preparation is described in General Procedure A) and 3-(2-Methoxy-ethyl)-2-(S)-pyrrolidin-2-yl-3H-imidazo[4,5-c]pyridine N-3 (preparation is described in General Procedure N; PG=Cbz) according to General Procedure B.

1H-NMR (CDCl₃, rotamers): δ 8.89 (s, 0.37H), 8.51 (s, 0.27H), 8.39-8.30 (m, 0.19H), 8.16-8.07 (m, 0.30H), 7.89-7.70 (m, 2.03H), 5.40-5.26 (m, 1H), 4.53-4.39 (m, 1.85H), 4.12-3.95 (m, 1.41H), 3.87-3.50 (m, 5.83H), 3.38-3.20 (m, 4.40H), 2.98-2.77 (m, 1.33H), 2.72-2.62 (m, 0.64H), 2.53-2.24 (m, 1.93H), 2.03-1.85 (m, 1.43H), 1.81-1.55 (m, 2.52H), 1.50-1.12 (m, 7.07H), 0.94-0.75 (m, 3.79H).

13C-NMR (CDCl₃, rotamers): δ 174.7, 174.3, 173.5, 161.4, 157.3, 155.4, 143.1, 132.3, 131.4, 131.2, 131.0, 113.1, 112.9, 71.2, 71.0, 60.2, 60.0, 59.3, 59.2, 58.4, 58.1, 53.2, 48.0, 46.5, 46.3, 43.8, 43.7, 33.8, 32.6, 31.8, 31.5, 29.3, 29.2, 22.9, 22.5, 22.4, 13.9.

ES-MS: calcd. for C₂₁H₃₁N₅O₄ (417.24); found (pos.): 418.7 [M+H].

### Example 88

### N-Hydroxy-N-(2-{2-[1-(2-methoxy-ethyl)-1H-imidazo[4,5-c]pyridin-2-yl]-pyrrolidine-1-carbonyl}-hexyl)-formamide

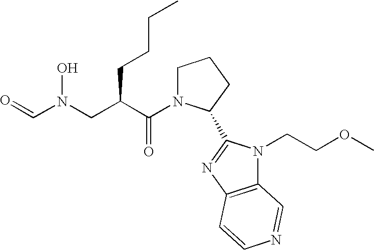

The title compound is prepared from (R)-2-[(Benzyloxy-formyl-amino)-methyl]-hexanoic acid A-7 (R2=n-butyl, preparation is described in General Procedure A) and 3-(2-Methoxy-ethyl)-2-(R)-pyrrolidin-2-yl-3H-imidazo[4,5-c]pyridine N-3 (preparation is described in General Procedure N; PG=Cbz) according to General Procedure B.

1H-NMR (CDCl₃, rotamers): δ 8.84 (s, 0.69H), 8.48 (d, J=5.6, 0.20H), 8.44-8.22 (m, 1.56H), 7.81 (s, 0.56H), 7.74 (s, 0.18H), 7.63 (d, J=5.6, 0.68H), 5.65-5.04 (m, 4.99H), 4.94-4.74 (m, 1H), 4.50-4.40 (m, 1.03H), 4.36-4.25 (m, 0.30H), 4.03-3.60 (m, 5.83H), 3.55-3.34 (m, 1.11H), 3.34-3.13 (m, 3.87H), 3.02-2.93 (m, 0.31H), 2.60-1.92 (m, 4.50H), 1.72-1.56 (m, 0.88H), 1.56-1.06 (m, 6.85H), 0.93-0.69 (m, 3.24H).

13C-NMR (CDCl₃, rotamers): δ 174.4, 172.3, 165.3, 161.6, 161.3, 156.2, 148.5, 142.6, 140.1, 139.7, 132.6, 132.4, 131.8, 131.6, 114.4, 113.7, 70.7, 59.3, 59.1, 54.7, 52.8, 51.0, 48.7, 47.8, 44.5, 43.9, 41.2, 33.4, 31.9, 31.8, 31.7, 30.0, 28.9, 28.7, 25.3, 22.9, 22.6, 21.9, 13.9, 13.8.

ES-MS: calcd. for C₂₁H₃₁N₅O₄ (417.24); found (pos.): 418.6 [M+H]; found (neg.): 416.2 [M−H].

### Example 89

### N-[2-Cyclopentylmethyl-3-(2-oxazolo[4,5-b]pyridin-2-yl)-pyrrolidine-1-yl)-3-oxo-propyl]-N-hydroxy-formamide

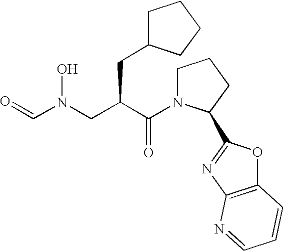

The title compound is prepared from (R)-2-[(Benzyloxy-formyl-amino)-methyl]-3-cyclopentyl-propionic acid A-7 (R2=cyclopentylmethyl, preparation is described in General Procedure A) and (S)-2-Pyrrolidin-2-yl-oxazolo[4,5-b]pyridine F-5 (preparation is described in General Procedure F) according to General Procedure B.

1H-NMR (CDCl₃, rotamers): δ 10.26 (s, 0.17H), 9.72 (s, 0.09H), 8.55-8.41 (m, 1.06H), 8.30-8.25 (m, 0.26H), 8.18 (s, 0.08H), 7.88-1.60 (m, 1.78H), 7.36-7.16 (m, 1.27H), 5.40-5.21 (m, 1H), 4.91-4.76 (m, 0.09H), 4.48-4.36 (m, 0.20H), 4.18-3.92 (m, 0.65H), 3.92-3.50 (m, 4.58H), 3.50-3.26 (m, 1.12H), 3.25-3.08 (m, 0.90H), 3.08-2.84 (m, 0.37H), 2.45-1.29 (m, 19.75H), 1.29-0.90 (m, 3.65H).

13C-NMR (CDCl₃, rotamers): δ 175.8, 173.7, 172.6, 169.8, 163.6, 162.4, 161.5, 156.8, 155.6, 147.5, 147.1, 146.5, 146.3, 142.7, 121.3, 120.6, 120.1, 119.9, 119.1, 118.8, 118.4, 118.3, 61.0, 58.9, 55.0, 54.8, 51.3, 47.1, 40.5, 37.9, 37.4, 37.2, 36.3, 36.1, 33.1, 33.0, 32.9, 32.7, 30.7, 25.2, 25.1, 24.7, 14.1.

ES-MS: calcd. for C₂₀H₂₆N₄O₄ (386.20); found (pos.): 387.7 [M+H]; found (neg.): 385.3.

### Example 90

### N-Hydroxy-N-[2-(4-fluoro-2-oxazolo[4,5-b]pyridin-2-yl)-pyrrolidine-1-carbonyl]-hexyl}-formamide

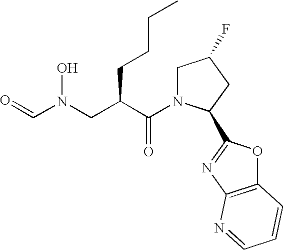

The title compound is prepared from (R)-2-[(Benzyloxy-formyl-amino)-methyl]-hexanoic acid A-7 (preparation is described in General Procedure A) and 2-((2S,4R)-4-Fluoro-pyrrolidin-2-yl)-oxazolo[4,5-b]pyridine F-5 (preparation is described in General Procedure F) according to General Procedure B.

1H-NMR (CDCl₃, rotamers): δ 10.82 (s, 0.19H), 8.65-8.49 (m, 1.41H), 8.34 (s, 0.20H), 8.21 (s, 0.08H), 8.08 (s, 0.07H), 8.00-7.96 (m, 0.07H), 7.92-7.70 (m, 2.11H), 7.41-7.22 (m, 1.64H), 7.05-6.99 (m, 0.06H), 5.78-5.64 (m, 0.27), 5.58-5.23 (m, 2.60H), 4.60-4.25 (m, 1H), 4.22-3.55 (m, 4.22H), 3.48-3.28 (m, 0.95H), 3.28-2.94 (m, 1.56H), 2.94-2.70 (m, 1.39H), 2.70-2.35 (m, 1.80H) 1.89-1.21 (m, 10.56H), 0.95-0.78 (m, 4.23H), 0.64-0.55 (m, 0.11H).

13C-NMR (CDCl₃, rotamers): δ 173.8, 172.7, 169.5, 168.3, 163.9, 161.7, 157.1, 155.3, 147.7, 147.2, 146.7, 146.6, 142.7, 122.4, 120.7, 120.4, 120.2, 118.7, 118.6, 92.9, 92.5, 90.7, 90.5, 54.2, 54.1, 54.0, 53.8, 53.5, 53.4, 53.3, 52.8, 50.9, 43.3, 42.6, 41.1, 38.0, 37.8, 29.7, 29.6, 28.8, 28.7, 22.8, 22.7, 22.6, 13.9, 13.8.

ES-MS: calcd. for C₁₈H₂₃FN₄O₄ (378.17); found (pos.): 379.4 [M+H]; found (neg.): 377.6.

### Example 91

### 2-(1-{2-[(Formyl-hydroxy-amino)-methyl]-hexanoyl}-pyrrolidin-2-yl)-benzooxazole-5-sulfonic acid amide

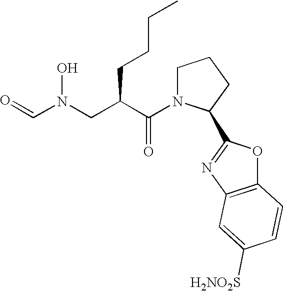

The title compound is prepared from (R)-2-[(Benzyloxy-formyl-amino)-methyl]-hexanoic acid A-7 (preparation is described in General Procedure A) and (S)-2-Pyrrolidin-2-yl-benzooxazole-5-sulfonic acid amide F-5 (preparation is described in General Procedure F) according to General Procedure B.

ESI-MS: calcd. for C₁₉H₂₆N₄O₆S (438.51); found (pos.): 439.4 [M+H]. found (neg.): 437.5[M−H].

### Example 92

### (R)-3-[(2S,4R)-4-Fluoro-2-(3H-imidazo[4,5-c]pyridin-2-yl)-pyrrolidine-1-carbonyl]-heptanoic acid hydroxyamide

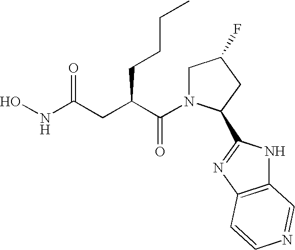

The title compound is prepared from (R)-2-Butyl-succinic acid 4-tert-butyl ester D-1 (as described in D) and 2-((2S,4R)-4-Fluoro-pyrrolidin-2-yl)-3H-imidazo[4,5-c]pyridine E-5 (preparation is described in General Procedure E) according to General Procedure D.

1H-NMR (MeOH-d₄, rotamers): δ 8.89-8.87 (s, 0.13H), 8.87-8.86 (s, 0.87H), 8.36-8.28 (m, 1.3H), 7.78-7.62 (dd, J=0.08, 1.31H), 5.6-5.52 (m, 0.65H), 5.5-5.4 (m, 0.73H), 5.38-5.28 (m, 1.27H), 4.42-4.2 (m, 1.33H), 4.2-4.02 (m, 1.3H), 3.17-3.04 (m, 1.28H), 2.85-2.69 (m, 1.48H), 2.63-2.52 (m, 0.85H), 2.52-2.41 (m, 0.82H), 2.41-2.32 (m, 1.18H), 2.22-2.13 (m, 1.05H), 1.59-1.46 (m, 1.58H), 1.46-1.28 (m, 2.4H), 1.28-1.07 (m, 4.87H), 0.96-0.87 (m, 0.76H), 0.85-0.65 (m, 3.5H). ES-MS: calcd. for C₁₈H₂₄FN₅O₃ (377.4); found (pos.): 378.4[M+H];

### Example 93

### 3-[2-(1H-Benzoimidazol-2-yl)-4-fluoro-pyrrolidine-1-carbonyl]-heptanoic acid hydroxyamide

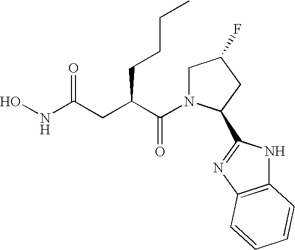

The title compound is prepared from (R)-2-Butyl-succinic acid 4-tert-butyl ester D-1 (as described in D) and 2-((2S,4R)-4-Fluoro-pyrrolidin-2-yl)-1H-benzoimidazole E-5 (preparation is described in General Procedure E) according to General Procedure D.

1H-NMR (MeOH-d₄, rotamers): δ 8.21 (s, 0.03H), 7.59-7.45 (m, 1.88H), 7.26-7.15 (m, 1.96H), 5.56-5.48 (m, 0.57H), 5.43-5.36 (m, 0.52H), 5.28 (t, J=8.6, 8.6, 0.92H), 4.37-4.24 (m, 0.96H), 4.13 (dd, J=3.1, 12.4, 0.45H), 4.04 (dd, J=3.2, 12.4, 0.45H), 3.76-3.62 (m, 0.25H), 3.25-3.18 (m, 0.17H), 3.13-3.02 (m, 0.91H), 2.99-2.84 (m, 0.43H), 2.79-2.65 (m, 1.01H), 2.63-2.49 (m, 1.31H), 2.49-2.28 (m, 0.87H), 2.19-2.08 (m, 0.85H), 2.08-1.96 (m, 0.32H), 1.59-1.44 (m, 1.13H), 1.44-1.26 (m, 2.26H), 1.26-1.04 (m, 3.56H), 0.95-0.85 (m, 0.53H), 0.81-0.72 (m, 2.55H). ¹³C-NMR (MeOH-d₄, rotamers): δ 176.6, 170.8, 156.4, 123.9, 123.6, 115.9, 94.1, 92.4, 55.3, 55.2, 41.3, 39.7, 39.4, 35.7, 33.1, 30.0, 23.7, 14.2.

ES-MS: calcd. for C₁₉H₂₅FN₄O₃ (376.19); found (pos.): 377.4 [M+H]; found (neg.): 375.5 [M−H].

### Example 94

### (R)-3-[(S)-2-(3H-Imidazo[4,5-c]pyridin-2-yl)-pyrrolidine-1-carbonyl]-heptanoic acid hydroxyamide

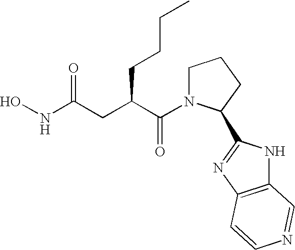

The title compound is prepared from (R)-2-Butyl-succinic acid 4-tert-butyl ester D-1 (as described in D) and (S)-2-Pyrrolidin-2-yl-3H-imidazo[4,5-c]pyridine E-5 (preparation is described in General Procedure E) according to General Procedure D. 1H-NMR (MeOH-d₄, rotamers): δ 8.95-8.9 (s, 0.2H), 8.9-8.6 (s, 0.77H), 8.4-8.3 (d, 1.14H), 8.2-8.1 (s, 0.48H), 7.8-7.6 (dd, 1.2H), 5.5-5.3 (m, 0.36H), 5.3-5.2 (m, 1.1H), 4.08-3.85 (m, 2.2H), 3.75-3.55 (m, 0.81H), 3.5-3.43 (m, 0.3H), 3.18-3.03 (m, 1.29H), 2.5-2.33 (m, 2.2H), 2.33-2.23 (m, 1.28H), 2.23-2.03 (m, 3H), 1.67-1.5 (m, 1.48H), 1.5-1.17 (m, 5.9H), 1.0-0.88 (m, 0.72H), 0.88-0.73 (m, 2.91H).

ES-MS: calcd. for C₁₈H₂₅N₅O₃ (359.4); found (pos.): 360.3[M+H];

### Example 95

### 3-[2-(5-Sulfamoyl-benzooxazol-2-yl)-pyrrolidine-1-carbonyl]-heptanoic acid hydroxyamide

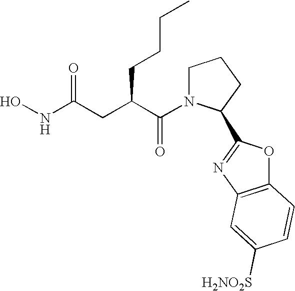

The title compound is prepared from (R)-2-Butyl-succinic acid 4-tert-butyl ester D-1 (as described in D) and (S)-2-Pyrrolidin-2-yl-benzooxazole-5-sulfonic acid amide F-5 (preparation is described in General Procedure F) according to General Procedure D.

ESI-MS: calcd. for C19H26N4O6S (438.51); found (pos.): 439.4 [M+H]. found (neg.): 437.5[M−H].

### Example 96

### 3-[2-(1H-Benzoimidazol-2-yl)-4-fluoro-pyrrolidine-1-carbonyl]-heptanoic acid

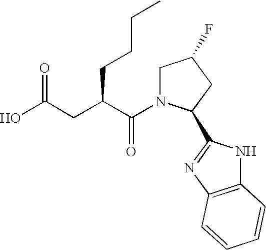

The title compound is prepared from (R)-2-Butyl-succinic acid 4-tert-butyl ester D-1 (as described in D) and 2-((2S,4R)-4-Fluoro-pyrrolidin-2-yl)-1H-benzoimidazole E-5 (preparation is described in General Procedure E; PG=CBz) according to General Procedure D.

1H-NMR (MeOH-d₄, rotamers): δ 7.77-7.72 (m, 1.71H), 7.72-7.65 (m, 0.12H), 7.57-7.51 (m, 1.89H), 7.47-7.41 (m, 0.10H), 5.70-5.64 (m, 0.04H), 5.62-5.56 (m, 0.49H), 5.48-5.43 (m, 0.50H), 5.48-5.43 (m, 0.97H), 4.53-4.39 (m, 0.96H), 4.18 (dd, J=3.1, 12.4, 0.51H), 4.09 (dd, J=3.2, 12.4, 0.50H), 3.09-3.00 (m, 1.04H), 2.97-2.84 (m, 1.01H), 2.67-2.40 (m, 3.12H), 1.99-1.96 (m, 0.04H), 1.58-1.46 (m, 1.12H), 1.46-1.04 (m, 5.31H), 0.94-0.88 (m, 0.21H), 0.86-0.77 (m, 2.81H). ¹³C-NMR (MeOH-d₄, rotamers): δ 178.3, 175.6, 155.2, 133.0, 127.3, 115.1, 94.0, 92.2, 55.5, 55.2, 54.2, 41.1, 39.6, 39.4, 36.9, 33.0, 30.1, 23.7, 14.2. ES-MS: calcd. for C₁₉H₂₄FN₃O₃ (361.18); found (pos.): 362.2 [M+H]; found (neg.): 360.6 [M−H].

### Example 97

### Inhibition of Peptide Deformylase Activity

A PDF/FDH coupled assay (Lazennec et al., Anal. Biochem., Vol. 224, pp. 180-182 (1997)) is used. In this coupled assay, the formate released by PDF from its substrate fMAS is oxidized by the coupling enzyme FDH, reducing one molecule of NAD⁺ to NADH, which causes an increase in absorption at 340 nM. All assays are carried out at room temperature in a buffer of 50 mM HEPES, pH 7.2, 10 mM NaCl, 0.2 mg/mL BSA, in half-area 96-well microtiter plates (Corning). The reaction is initiated by adding a mixture of 0.5 Unit/mL FDH, 1 mM NAD⁺, and fMAS at the desired concentration. To determine IC₅₀ (the concentration needed to inhibit 50% of enzyme activity) values, PDF is pre-incubated for 10 minutes with varying concentrations of the inhibitor, and the deformylation reaction is initiated by the addition of reaction mixture containing 4 mM fMAS. The initial reaction velocity, y, is measured as the initial rate of absorption increase at 340 nM using a SpectraMax plate reader (Molecular Devices, Sunnyvale, Calif.). The inhibitor concentration [ln] at which 50% of the enzyme activity is inhibited, IC₅₀, is calculated using the following formula:

y=yₒ/(1+[ln]/IC₅₀)

where yₒ is the reaction velocity in the absence of inhibitor. Solving this equation for IC₅₀ at the [ln] when y=yₒ/2 yields IC₅₀. The IC₅₀ is calculated based on a nonlinear least-square regression fit using a commercial software package (Deltapoint, Inc., Chicago, Ill.). Using this assay, the IC₅₀ of various compounds are determined. The IC₅₀ for the various compounds is determined against deformylase enzyme containing nickel and zinc as the metal ion. The IC₅₀ values of preferred compounds of formula (I) determined for the zinc-containing deformylase range from about 0.001 μM to about 0.2 μM. The IC₅₀ values of preferred compounds of formula (I) determined for the nickel-containing deformylase range from about 0.005 μM to about 3 μM.

### Example 98

### Assay for Testing Antimicrobial Activity

Minimum inhibitory concentrations (MICs) are determined using the microdilution method in 96-well format plates. Compounds are suspended in DMSO at 5 or 10 mg/mL and stored at 4° C. until used. They are diluted in Mueller-Hinton Broth (MHB) or Trypticase Soy Broth (TSB) and used for MIC determination. The range of concentrations tested is 64-0.0625 μg/mL final concentration using a two-fold dilution system.

The inoculum is prepared from cells grown on Trypticase Soy Agar (TSA) and incubated overnight at 35° C., 5-10 colonies are used to inoculate MHB or TSB broths, and the culture is incubated overnight at 35° C. The overnight culture is diluted 1:10, incubated for 1 hour at 35° C., diluted to the appropriate inoculum size and applied to the wells containing broth and test compound. Inoculum sizes are 2×10⁴ CFU/mL.

Plates are incubated at 35° C. for 48 hours and MIC are recorded after 18 hours of incubation for bacteria. MIC is defined as the lowest concentration of compound that does not produce visible growth after incubation. The deformylase enzyme is obtained from E. coli.

The MICs for the compounds of formula (I) are below 2 μg/mL, preferably below 1.5 μg/mL, more preferably below 1 μg/mL, most preferably below 0.5 μg/mL.

The following are representative pharmaceutical formulations containing a compound of the present invention:

### Example 99

### Tablet Formulation

The following ingredients are mixed intimately and pressed into single scored tablets:

| Ingredient | Amount (mg) |
|---|---|
| Compound of this invention | 400 |
| Cornstarch | 50 |
| Croscarmellose sodium | 25 |
| Lactose | 120 |
| Magnesium stearate | 5 |

### Example 100

### Capsule Formulation

The following ingredients are mixed intimately and loaded into a hard-shell gelatin capsule:

| Ingredient | Amount (mg) |
|---|---|
| Compound of this invention | 200 |
| Lactose, spray - dried | 148 |
| Magnesium stearate | 2 |

### Example 101

### Suspension Formulation

The following ingredients are mixed to form a suspension for oral administration:

| Ingredient | Amount (g or mL) | |
|---|---|---|
| Compound of this invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.00 | g |
| Veegum K (Vanderbilt Co.) | 1.0 | g |
| Flavoring | 0.035 | mL |
| Colorings | 0.5 | mg |
| Distilled water | q.s. to 100 | mL |

### Example 102

### Injectable Formulation

The following ingredients are mixed to form an injectable formulation:

| Ingredient | Amount (mg or mL) | |
|---|---|---|
| Compound of this invention | 0.2-20 | mg |
| Sodium acetate buffer solution, 0.4 M | 20 | mL |
| HCl (1 N) or NaOH (1 N) | q.s. to suitable pH | |
| Water (distilled, sterile) | q.s. to 20 | mL |

### Example 103

### Suppository Formulation

A suppository of total weight 2.5 g is prepared by mixing the compound of the invention with Witepsol® H-5 (triglycerides of saturated vegetable fatty acid; Riches-Nelson, Inc., New York), and has the following composition:

| Ingredient | Amount (mg) |
|---|---|
| Compound of this invention | 500 mg |
| Witepsol ® H-15 | Balance |

### INDUSTRIAL APPLICABILITY

The N-formyl hydroxylamine compounds of this invention are inhibitors of peptidyl deformylase and may therefore be used in various medicinal applications, including treating disorders amenable to treatment by peptidyl deformylase inhibitors, for example treatment of bacterial infections such as tuberculosis.

1. A compound of formula (I) or a pharmaceutically acceptable salt, ester or prodrug thereof: 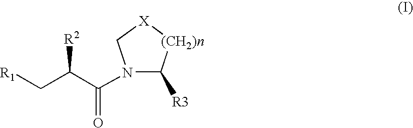
wherein
n is 1 or 2;
X is CH₂, S or CHF;
R1 is —N(OH)CHO or —C(O)NH(OH) R2 is alkyl, alkylcycloalkyl or alkylaryl or R2 represents a cycloalkyl group, where the carbon adjacent to the carbonyl group forms part of the cycloalkyl ring;
R3 is a substituent of formula (a) or (b), or tetrazolyl, 2-perimidinyl or 4-phenylimidazol-2-yl; 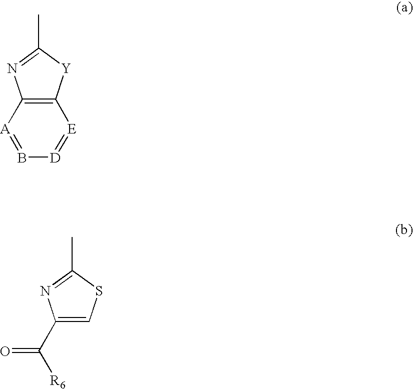
where
Y is NH, O, S or NR4;
A, B, D and E are each independently selected from CH, N, or CR5; or A and E are CH and B and D are fused to and form part of an aryl ring or a 5- or 6-membered nitrogen heterocycle;
R4 is hydroxyalkyl, alkyl or heteroalkyl;
R5 is haloalkyl, heterocyclo optionally substituted with an alkyl group, halogen, alkyl, amino, cyano, nitro, aryl, alkoxy, haloalkoxy, —CO₂R7, —SO₂R8, —NHC(O)R9 or —NHSO₂R9; or two R5 groups together form a 6-membered oxygen containing heterocycle, optionally substituted with one or more halogens and fused to the 6-membered ring of substituent (a);
R6 is amino or alkoxy;
R7 is H, alkyl, NHR10, NR10R11 or NH₂;
R8 is aryl, heterocyclo, alkyl or amino;
R9 is heteroaryl or aryl; and
R10 and R11 are each independently an alkyl, alkenyl, alkynyl or aryl group.

2. A compound as claimed in claim 1 or a pharmaceutically acceptable salt, ester or prodrug thereof wherein n is 1.

3. A compound as claimed in claim 1 or a pharmaceutically acceptable salt, ester or prod rug thereof wherein R1 is —N(OH)CHO.

4. A compound as claimed in claim 1 or a pharmaceutically acceptable salt, ester or prodrug thereof where X is CH₂ or CHF.

5. A compound as claimed in claim 1 or a pharmaceutically acceptable salt, ester or prodrug thereof wherein R3 is a substituent of formula (a).

6. A compound as claimed in claim 1 or a pharmaceutically acceptable salt, ester or prodrug thereof wherein R2 is lower alkyl, lower alkylcycloalkyl or lower alkyaryl.

7. A compound as claimed in claim 6 or a pharmaceutically acceptable salt, ester or prodrug thereof wherein R2 is n-propyl, n-butyl, n-pentyl, cyclopentylmethyl or benzyl, or where R2 is a cyclohexyl group, where the carbon adjacent to the carbonyl group forms part of the cyclohexyl ring.

8. A compound as claimed in claim 7 or a pharmaceutically acceptable salt, ester or prodrug thereof wherein R2 is n-butyl.

9. A compound as claimed in claim 1 or a pharmaceutically acceptable salt, ester or prodrug thereof wherein R3 is a substituent of formula (a) and Y is O or NH.

10. A compound as claimed in claim 1 or a pharmaceutically acceptable salt, ester or prodrug thereof wherein R3 is a substituent of formula (a) and R5 is trifluoromethyl, 4-Me-piperizin-1-yl, fluoro, chloro, methoxy, amino, methyl, cyano, t-butyl, phenyl, nitro, trifluoromethoxy, —SO₂NH₂, —SO₂(morpholino), SO₂Et, —CO₂Me, —CO₂Et, —NHC(O)(2-pyrazinyl) or —NHSO₂Ph, or two R5 groups together form a substituent (i) or (ii):

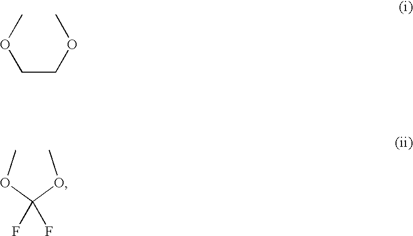

11. A compound as claimed in claim 1 or a pharmaceutically acceptable salt, ester or prodrug thereof wherein Band D are fused to a phenyl ring or a pyrazole ring.

12. A compound as claimed in claim 1 or a pharmaceutically acceptable salt, ester or prodrug thereof wherein R3 is a substituent of formula (b) and R6 is amino or ethoxy.

13. A compound as claimed in claim 1 or a pharmaceutically acceptable salt, ester or prodrug thereof wherein R4 is an alkyl group having an alkoxy substituent.

14. A compound as claimed in claim 1 or a pharmaceutically acceptable salt, ester or prod rug thereof wherein R4 is hydroxyethyl, methoxyethyl or methyl.

15. A compound of formula (I′), or a pharmaceutically acceptable salt, ester or prodrug thereof: 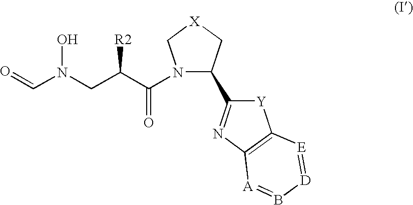
wherein
R2 is n-propyl, n-butyl, n-pentyl, cyclopentylmethyl, or benzyl;
X is CH2 or CHF;
Y is NH, O or S; and
A, B, D and E are each independently CH, N, or CR5;
where R5 is as defined in claim 1.

16. A compound as claimed in claim 15, or a pharmaceutically acceptable salt, ester or prodrug thereof, wherein R2 is n-butyl.

17. A compound as claimed in claim 15, or a pharmaceutically acceptable salt, ester or prodrug thereof, wherein Y is NH or O.

18. A compound as claimed in claim 1, or a pharmaceutically acceptable salt, ester or prodrug thereof, wherein A is N.

19. A compound as claimed in claim 1, or a pharmaceutically acceptable salt, ester or prodrug thereof, wherein Band E are both N.

20. A compound as claimed in claim 1, or a pharmaceutically acceptable salt, ester or prodrug thereof, wherein X is CHF.

21. A compound as claimed in claim 1, or a pharmaceutically acceptable salt, ester or prodrug thereof, wherein X is CHF.

22. A pharmaceutical composition comprising a compound as claimed in claim 1, or a pharmaceutically acceptable salt, ester or prodrug thereof, in combination with a pharmaceutically acceptable excipient, diluent or carrier.

23. A method for treating and/or preventing a disease or disorder amenable to treatment by peptidyl deformylase inhibitors comprising administering to a subject in need thereof an effective peptidyl deformylase inhibiting amount of a compound according to any claim 1, a pharmaceutically acceptable salt, ester or prodrug thereof.

24. A method as claimed in claim 23, wherein the disease or disorder is a bacterial infection.

25. A method as claimed in claim 24, wherein the bacterial infection is a mycobacterial infection.

26. A method as claimed in claim 25, wherein the mycobacterial infection is caused by Mycobacterium tuberculosis.

27. A method as claimed in claim 26, wherein the mycobacterial infection is caused by a multidrug resistant form of Mycobacterium tuberculosis.
